SEQUENCE LISTING (10 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 5 aa, synthetic construct
NYYMY

SEQ ID NO: 5 - protein, 17 aa, synthetic construct
GINPSNGGTNFNEKFKN

SEQ ID NO: 6 - protein, 11 aa, synthetic construct
RDYRFDMGFDY

SEQ ID NO: 7 - protein, 111 aa, synthetic construct [sequence omitted]

SEQ ID NO: 8 - protein, 120 aa, synthetic construct [sequence omitted]

SEQ ID NO: 9 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0193350 A1
(43) Pub. Date: Jul. 9, 2026

(54) METHODS FOR PREPARING ANTI-HUMAN PD-1 ANTIBODY CRYSTALS AND METHODS OF USE THEREOF

(71) Applicants: Merck Sharp & Dohme LLC, Rahway, NJ (US); Werthenstein Biopharma GmbH, Schachen (CH)

(72) Inventors: Paul Reichert, Montville, NJ (US); Chakravarthy Nachu Narasimhan, Scotch Plains, NJ (US); Andrew Bochnovich, Brooklyn, NY (US); Divya Chandra, Scotch Plains, NJ (US); Rebecca A. Chmielowski, Clark, NJ (US); Lorenzo Codan, Olten (CH); Aaron S. Cote, West Windsor, NJ (US); Ashley E. Lay-Fortenbery, Hoboken, NJ (US); Patrick Larpent, Lucerne (CH); Zhao Li, Green Brook, NJ (US); Mikhail D. Murashov, Toms River, NJ (US); Jordan R. Rampolla, Perry Hall, MD (US); Yongchao Su, Hillsborough, NJ (US)

(73) Assignees: Merck Sharp & Dohme LLC, Rahway, NJ (US); Werthenstein Biopharma GmbH, Schachen (CH)

(21) Appl. No.: 19/131,053

(22) PCT Filed: Nov. 20, 2023
(86) PCT No.: PCT/US2023/080455

§ 371 Date: May 19, 2025

Related U.S. Application Data
(60) Provisional application No. 63/426,949, filed on Nov. 21, 2022.
(60) Provisional application No. 63/580,110, filed on Sep. 1, 2023.

(51) Int. Cl. C07K 16/28; A61K 39/00

(52) U.S. Cl. CPC C07K 16/2818; A61K 2039/505; C07K 2317/24; C07K 2317/94

(57) ABSTRACT

The invention provides methods for producing high concentration crystalline drug substances of an anti-PD-1 mono-clonal antibody (mAb), wherein the mAb is pembrolizumab or a pembrolizumab variant. The invention further relates to pharmaceutical compositions comprising the crystals produced by the methods herein and methods of use thereof.

### FIELD OF THE INVENTION

The invention relates to methods for producing high concentration crystalline suspensions of anti-PD-1 monoclonal antibodies. The invention further relates to pharmaceutical compositions comprising the crystals produced by the methods herein and methods of use thereof.

### CROSS-REFERENCE TO RELATED APPLICATIONS

This application claims the benefit of priority to U.S. Provisional Patent Application No. 63/426,949, filed Nov. 21, 2022 and U.S. Provisional Patent Application No. 63/580,110 filed Sep. 1, 2023, the disclosures of which are hereby incorporated by reference in its entirety.

### BACKGROUND OF THE INVENTION

Therapeutic and diagnostic antibodies have become the fastest growing area of the biopharmaceutical industry. A critical aspect to the success of antibodies as therapeutic agents is the development of improved methods to express, purify and characterize these proteins. In general, antibody therapeutics are large (typically >150 kDa) and complex in nature and must be administered in stoichiometric rather than catalytic quantities. Production and purification scales have thus reached levels of production that were previously assumed impossible. There is also a need for the development of stable formulations and delivery strategies for such large amounts of a complex molecule.

Development of stable formulations comprising a high concentration of active agent, such as an antibody or antigen-binding fragment, is particularly important for biological formulations intended for subcutaneous administration to a patient, since the volume of solution delivered to a patient is greatly reduced. Subcutaneous administration is the preferred method of administration of many antibodies, in part because it may enable self-administration or easier administration by a medical professional (e.g., pharmacist, doctor, or nurse). Therapeutic antibodies are traditionally prepared in lyophilized form or in solution. Lyophilized forms may exhibit enhanced long-term stability, but require reconstitution prior to use, making them less than ideal for self-administration. On the other hand, stable liquid formulations are more challenging to develop and often require cold storage prior to use.

Immune checkpoint therapies targeting the PD-1 axis have resulted in groundbreaking improvements in clinical response in multiple human cancers (Brahmer et al., N Engl J Med 2012, 366:2455-65; Garon et al. N Engl J Med 2015, 372:2018-28; Hamid et al., N Engl J Med 2013, 369:134-44; Robert et al., Lancet 2014, 384:1109-17; Robert et al., N Engl J Med 2015, 372:2521-32; Robert et al., N Engl J Med 2015, 372:320-30; Topalian et al., N Engl J Med 2012, 366:2443-54; Topalian et al., J Clin Oncol 2014, 32:1020-30; Wolchok et al., N Engl J Med 2013, 369:122-33). Immune therapies targeting the PD-1 axis include monoclonal antibodies directed to the PD-1 receptor (KEYTRUDA® (pembrolizumab), Merck Sharp & Dohme LLC, Rahway, NJ, USA and OPDIVO™ (nivolumab), Bristol-Myers Squibb Company, Princeton, NJ, USA) and also those that bind to the PD-L1 ligand (MPDL3280A; TECENTRIQ™ (atezolizumab), Genentech, San Francisco, CA, USA; IMFINZI™ (durvalumab), AstraZeneca Pharmaceuticals LP, Wilmington, DE; BAVENCIO™ (avelumab), Merck KGaA, Darmstadt, Germany). Both therapeutic approaches have demonstrated anti-tumor effects in numerous cancer types.

Crystallization is currently one of the most powerful techniques used for purifying and isolating small organic active pharmaceutical ingredients (API) at the manufacturing scale. The crystallization of mAbs has historically been considered unrealizable at manufacturing scale. The characterization of crystalline proteins is complex and remains rather limited due to inherently challenging physical properties of the molecules.

### SUMMARY OF THE INVENTION

The disclosure provides an improved high concentration stable formulation of anti-PD-1 antibodies for use, e.g., in the treatment of patients with cancer. Specifically, the disclosure enables administration of a high concentration crystalline solution with sufficiently low viscosity and low aggregation to be conveniently delivered.

In one aspect, the invention relates to a method for producing a high concentration crystalline suspension of an anti-PD-1 monoclonal antibody (mAb) comprising: (a) mixing: (i) an aqueous buffered solution comprising about 150 mg/mL to about 300 mg/ml of the mAb, wherein the anti-PD-1 mAb is pembrolizumab or a pembrolizumab variant, and (ii) an additive selected from the group consisting of caffeine, theophylline, 2′ deoxyguanosine-5′-monophosphate, a bioactive gibberellin, and a pharmaceutically acceptable salt of said bioactive gibberellin; and (b) incubating the crystallization solution for a period of time sufficient for crystal formation.

In one aspect, the invention relates to a method for producing a high concentration crystalline suspension of an anti-PD-1 monoclonal antibody (mAb) comprising: (a) concentrating the aqueous buffered solution to allow for the high concentration solution; (b) mixing: (i) an aqueous buffered solution comprising about 150 mg/mL to about 300 mg/mL of the mAb, wherein the anti-PD-1 mAb is pembrolizumab or a pembrolizumab variant, and (ii) an additive selected from the group consisting of caffeine, theophylline, 2′ deoxyguanosine-5′-monophosphate, a bioactive gibberellin, and a pharmaceutically acceptable salt of said bioactive gibberellin; and (c) incubating the crystallization solution for a period of time sufficient for crystal formation.

In one aspect, the invention relates to a method for producing a high concentration crystalline suspension of an anti-PD-1 monoclonal antibody (mAb) comprising: (a) mixing: (i) an aqueous buffered solution comprising about 150 mg/mL to about 300 mg/mL of the mAb, wherein the anti-PD-1 mAb is pembrolizumab or a pembrolizumab variant, and (ii) an additive selected from the group consisting of caffeine, theophylline, 2′ deoxyguanosine-5′-monophosphate, a bioactive gibberellin, and a pharmaceutically acceptable salt of said bioactive gibberellin, (b) concentrating the aqueous buffered solution to allow for the high concentration solution; and (c) incubating the crystallization solution for a period of time sufficient for crystal formation.

In one embodiment, the invention relates to a method for producing a high concentration crystalline anti-PD-1 monoclonal antibody (mAb) comprising: (a) concentrating the aqueous buffered solution to allow for the high concentration solution; (b) mixing: (i) an aqueous buffered solution comprising about 150 mg/mL to about 300 mg/mL of the mAb, wherein the anti-PD-1 mAb is pembrolizumab or a pembrolizumab variant, (ii) an additive selected from the group consisting of caffeine, theophylline, 2′ deoxyguanosine-5′-monophosphate, a bioactive gibberellin, and a pharmaceutically acceptable salt of said bioactive gibberellin, and (iii) optionally, polyethylene glycol (PEG); to form a crystallization solution; (c) incubating the crystallization solution for a period of time sufficient for crystal formation; and (d) optionally harvesting the crystalline anti-PD-1 mAb from the solution.

In one embodiment, the invention relates to a method for producing a high concentration crystalline anti-PD-1 monoclonal antibody (mAb) comprising (a) mixing: (i) an aqueous buffered solution comprising about 150 mg/mL to about 300 mg/mL of the mAb, wherein the anti-PD-1 mAb is pembrolizumab or a pembrolizumab variant, (ii) an additive selected from the group consisting of caffeine, theophylline, 2′ deoxyguanosine-5′-monophosphate, a bioactive gibberellin, and a pharmaceutically acceptable salt of said bioactive gibberellin, and (iii) optionally, polyethylene glycol (PEG); to form a crystallization solution, (b) concentrating the aqueous buffered solution on a filter to allow for the high concentration solution; (c) incubating the crystallization solution for a period of time sufficient for crystal formation; and (d) optionally harvesting the crystalline anti-PD-1 mAb from the solution.

In some embodiments, the mAb is pembrolizumab. In further embodiments, the mAb is a pembrolizumab variant that maintains the ability to bind to PD-1 and the ability to bind to the precipitant solution.

In one aspect, the invention relates to a composition comprising about 150 mg/ml to about 300 mg/ml of the isolated anti-PD-1 crystals made by the methods of the invention.

In some embodiments, the crystallization solution further comprises about 1% to about 10% dextran sodium sulfate (DSS).

In one aspect, the invention provides methods of treating cancer and/or infectious disease by administering the high concentration compositions of the invention to a patient in need thereof. In specific embodiments, the compositions are administered to the patient via intravenous infusion or subcutaneous injection. In alternative embodiments, the crystals are delivered to the patient by inhalation or insufflation.

### BRIEF DESCRIPTION OF THE DRAWINGS

FIG. 1 depictscrystalline pembrolizumab derived from the process of Example 1. 8.4 mg of the resulting cake is shown in the left panel, reconstituted with 10 mM HEPES pH 7.0 for 10 minutes at room temperature. The dissolved cake is represented in the middle panel of FIG. 1. 2.5% caffeine in 10 mM histidine solution was subsequently added and mixed for two hours at room temperature for crystallization. The crystalline pembrolizumab suspension in the right panel of FIG. 1 was measured at 133 mg/ml.

FIGS. 2A-2C show images of SONICC characterization of crystalline pembrolizumab suspensions derived from Example 1. Images show crystals made following incubation of the crystallization solution, characterized using the visible (FIG. 2A), UV TPEF (FIG. 2B), and SHG mode (FIG. 2C) of the SONICC™ imaging system, respectively.

FIGS. 3A-3C show images of SONICC analyses of the crystalline pembrolizumab suspension derived from Example 2 showing the visible image in FIG. 3A, the UV-TPEF image in FIG. 3B, and the SHG image in FIG. 3C.

FIGS. 4A-4F show images of SONICC analyses (Example 3B) of the crystalline pembrolizumab suspension derived from Example 3A. FIG. 4A, FIG. 4B, and FIG. 4C show a sample from the PEG solution indicating an amorphous solid whereas FIG. 4D, FIG. 4E, and FIG. 4F show a sample taken from the PEG and caffeine suspension consistent with a crystalline suspension. Visible images are provided in FIG. 4A and FIG. 4D, UV-TPEF images in FIG. 4B and FIG. 4E, and SHG images in FIGS. 4C and 4F.

FIGS. 5A-5F show images of SONICC analyses (Example 6B) of the crystalline pembrolizumab suspension derived from Example 6A and shows SONICC results at 200 mg/ml at 300× at 1 mL scale. FIG. 5A, FIG. 5B, and FIG. 5C shows the crystal slurry drug product 1.0% PEG 3350 and FIG. 5D, FIG. 5E, and FIG. 5F shows the crystal slurry drug product 0.0% PEG 3350. Visible images are provided in FIG. 5A and FIG. 5D, UV-TPEF images in FIG. 5B and FIG. 5E, and SHG images in FIGS. 5C and 5F.

FIGS. 6A-6F show images of SONICC analyses (Example 7B) of the crystalline pembrolizumab suspension derived from Example 7A. FIG. 6A, FIG. 6B, and FIG. 6C shows crystalline drug product 0% PEG 3350. FIG. 6D, FIG. 6E, and FIG. 6F shows crystalline drug product control that is made by the low concentration process (13 mg/ml) as described in PCT application WO2020/092233. Visible images are provided in FIG. 6A and FIG. 6D, UV-TPEF images in FIG. 6B and FIG. 6E, and SHG images in FIGS. 6C and 6F.

FIGS. 7A and 7B show ¹³C cross polarization (CP) magic angle spinning (MAS) spectra of crystalline pembrolizumab grown without (top-FIG. 7A) and with (bottom-FIG. 7B) PEG. The spectra exhibit a high resolution, as indicated by a representative linewidth of 29 Hz.

FIGS. 8A and 8B show enlarged ¹³C cross polarization (CP) magic angle spinning (MAS) spectral comparison between the crystalline pembrolizumab grown without (FIG. 8A) and with (FIG. 8B) PEG. All peak positions in the spectra of the two samples are identical, identifying the highly similar protein conformation and crystallinity.

FIGS. 9A-9F show SONICC analyses for Example 8A. 30 mL crystalline pembrolizumab (HIS/Hepes) suspension shown in FIG. 9A, FIG. 9B, FIG. 9C vs. crystalline (HIS/Hepes) 1:1 PEG is shown in FIG. 9D, FIG. 9E, FIG. 9F. Visible images are provided in FIG. 9A and FIG. 9D, UV-TPEF images in FIG. 9B and FIG. 9E, and SHG images in FIGS. 9C and 9F.

FIGS. 10A-10C show three line graphs showing three-month stability data for the three different crystalline pembrolizumab suspensions as compared to the liquid formulation for Example 8. FIG. 10A shows the percent of high molecular weight species for exemplified suspensions at 5° C. FIG. 10B shows the percent of high molecular weight species for exemplified suspensions at 25° C. FIG. 10C shows the percent of high molecular weight species for exemplified suspensions at 40° C.

FIGS. 11A-11F show SONICC analyses of the crystalline pembrolizumab suspension derived from Example 9. 206 mg/ml at 30 ml scale crystalline pembrolizumab drug substance shown in FIG. 11A, FIG. 11B, and FIG. 11C. Crystalline pembrolizumab drug product shown in FIG. 11D, FIG. 11E, FIG. 11F. The visible image is shown in FIG. 11A and FIG. 11D, the UV-TPEF image in FIG. 11B and FIG. 11E, and the SHG image in FIGS. 11C and 11F.

FIG. 12 shows the viscosity characterization of drug product 1 in Example 24 comparing viscosity of drug product 1 (mPa-s) vs. time (seconds).

FIG. 13 shows the break-loose force and glide-force from two samples in Example 9E. The top line represents the glide force, whereas the bottom line represents the break loose force.

FIG. 14 is a line graph of the release profiles of representative crystalline and liquid formulations as described in Example 9F. Crystalline pembrolizumab drug product 1 derived from Example 9B was compared to a pembrolizumab substance at 165 mg/ml.

FIGS. 15A and 15B show the residual host cell protein (ppm) DNA (ppb) clearance via crystallization for pembrolizumab crystallization using two different crystallization methods with different conditions (PEG-Caffeine and Ammonium Phosphate) and three different in-process pools, namely Protein A product (PAP), Filtered Neutralized Viral Inactivated Pool (FNVIP) and AEX Product (AEXP). FIGS. 15A and 15B compare Feed, DisXtal-Ammonium Phosphate and DisXtal-Peg3350+caffeine conditions.

FIG. 16 is a line graph of the synchrotron small-angle X-ray scattering traces of two types of produced crystals. The graph shows the intensitites of the peaks plotted versus the momentum transfer (or s). FIG. 16 shows the synchrotron SAXS patterns of crystalline Phase 1 (solid line) and crystalline Phase 2 (dashed line).

FIG. 17 is a line graph showing the laboratory small-angle X-ray scattering traces of two types of produced crystals. The graph shows the intensitites of the peaks plotted versus the momentum transfer (or s). FIG. 17 shows the laboratory SAXS patterns of crystalline Phase 1 (solid line) and crystalline Phase 2 (dashed line).

FIGS. 18A and 18B show 1D ¹³C CP MAS spectra of pembrolizumab crystalline phase 1 (solid line) and of pembrolizumab crystalline phase 2 (dashed line) samples. FIG. 18B depicts an enlarged spectral regions of the spectra in FIG. 18A.

FIG. 19 shows a line graph of synchrotron SAXS traces of a representative suspension of pembrolizumab crystalline Phase 1 as described in Example 14. Line ‘A’ represents a suspension of pembrolizumab crystalline phase I as crystallized from an aqueous pH buffer solution containing L-histidine, caffeine, PEG3350 and DSS. Line ‘B’ represents a suspension of pembrolizumab crystalline phase I filtered and resuspended in an aqueous pH buffer solution containing PEG3350 and caffeine. Line ‘C’ represents a suspension of pembrolizumab crystalline phase I filtered and resuspended in an aqueous pH buffer solution containing caffeine. Line ‘D’ represents a suspension of pembrolizumab crystalline phase I filtered and resuspended in an aqueous pH buffer only.

FIG. 20 shows a line graph of synchrotron SAXS traces of a representative suspension of pembrolizumab crystalline Phase 2 as described in Example 14D. Line ‘A’ represents a suspension of pembrolizumab crystalline phase 2 as crystallized from an aqueous pH buffer containing L-histidine, caffeine and PEG3350. Line ‘B’ represents a suspension of pembrolizumab crystalline phase 2 as filtered and resuspended in an aqueous pH buffer solution containing caffeine. Line ‘C’ represents a suspension of pembrolizumab crystalline phase 2 as filtered and resuspended in an aqueous pH buffer solution only.

FIG. 21A is a line graph showing the filtrate collected for unseeded isothermal crystal, first semi-continuous crystal, and second semi-continuous crystal filtered at a pressure of 0.9 bars pressure. FIG. 21B is a line graph showing the filtrate collected for unseeded isothermal crystal, first semi-continuous crystal, and second semi-continuous crystal filtered at a pressure of 0.4 bars pressure.

FIG. 22 is a line graph showing the filtrate collected for Example 16B comparing unseeded isothermal crystallization with a crystal from emulsion and the amount of filtrate collected over time.

FIG. 23 is a line graph showing the viscosity of UFP comparing the impact of amino acids (histidine, arginine, lysine, no excipient) on pembrolizumab UFP concentration.

FIG. 24A and FIG. 24B are two line graphs showing the pharmacokinetics (PK) of human anti-PD-1 antibody, pembrolizumab, in naïve female minipigs that were evaluated for the crystalline formulation (FIG. 24B) and liquid formulation (FIG. 24A) after subcutaneous administration.

### DETAILED DESCRIPTION OF THE INVENTION

The invention provides high concentration pharmaceutical formulations comprising crystalline forms of pembrolizumab antibodies, and variants thereof. High concentration pembrolizumab crystalline suspensions were obtained wherein the protein concentration is about 150 mg/ml to about 300 mg/ml. The present invention also provides methods for preparing said high concentration monoclonal antibody (mAb) crystalline suspensions, wherein the mAb is pembrolizumab or a variant thereof, e.g., using bulk crystallization (batch and dialysis) in high yield, wherein the crystalline suspensions are made using a concentrating and incubating step.

The invention provides multiple methods with different conditions for preparing a stable pembrolizumab crystalline suspension at a concentration of about 150 mg/ml to about 300 mg/ml.

The invention further provides a composition/formulation comprising a stable pembrolizumab crystalline suspension at a concentration of about 150 mg/ml to about 300 mg/ml.

The invention further provides a crystallization method comprising caffeine as an additive, wherein the method comprises mixing an aqueous buffered solution comprising a high concentration mAb and an additive to form a crystallization solution, concentrating mAb, mixing the aqueous crystallization solution and incubating the crystallization solution for a period of time. Caffeine is added to the crystallization process at 4-30° C.

The invention further provides characterization of the crystalline suspensions using biochemical, rheological, and in-vitro release methods. The crystalline drug substance shows acceptable stability, rheological and injectability properties as well as in-vitro drug release profile. The studies showed the properties of the re-dissolved crystals (soluble anti-PD-1) are consistent with the intact antibody starting sample in bio-physical characterization studies.

In one aspect, the invention relates to methods established to prepare crystalline suspensions by bulk crystallization methods (batch) in high yield at 4-30° C., wherein the crystalline suspensions are prepared using a method comprising mixing an aqueous buffered solution comprising a high concentration mAb and an additive to form a crystallization solution, incubating the crystallization solution for a period of time, resulting in higher concentration of crystalline mAb than prior methods. In one further aspect, the mAb is concentration prior to mixing the aqueous buffered solution. In yet a further aspect, the mAb is concentrated by ultrafiltration. In a further aspect, the mAb is diafiltered with the aqueious buffered solution.

In another aspect, the invention relates to methods established to prepare crystalline suspensions by bulk crystallization methods (batch) in high yield at 4-30° C., wherein the crystalline suspensions are prepared using a method comprising mixing an aqueous buffered solution comprising a high concentration mAb, concentration the mAb on a filter, adding an additive to form a crystallization solution, and incubating the crystallization solution for a period of time, resulting in higher concentration of crystalline mAb than prior methods.

The resulting crystalline suspensions had a particle size of 0.5-1.0 microns. The resulting crystalline suspension if PEG is used together with caffeine has a particule size in the 5-25 μm range. These methods are amenable to multiple pharmaceutical applications such as purification (manufacture), storage, formulation, drug delivery and structure determination.

Most importantly, these conditions demonstrate a scalable one-step crystallization process which results in high concentration crystalline anti PD-1 mAb suspensions that can be formulated to have room temperature stability.

Formulations comprising a high concentration of crystalline anti-PD-1 antibodies made by the methods of the invention have several advantageous properties for use in therapy. Such high concentration formulations can enable more efficient administration to subject, e.g., by subcutaneous injection. Liquid solution formulations at 100 mg/ml cannot be used to deliver more than 100 mg to a subject with a single subcutaneous injection due to limitations of how much volume can be practically delivered at a single injection site. This limits dosing to approximately 1.5 mg/kg unless the subject is willing to accept (and in some cases administer) multiple injections at multiple sites. The crystalline suspensions of the present invention, in contrast, can be used to prepare pharmaceutical formulations up to 300 mg/ml or more, up to 500 mg/ml, enabling higher dosing with lower injection volume, and thus less discomfort. The crystalline suspensions injectability profiles are better than high concentration solution formulations (16 centipoise versus 40 centipoise), facilitating administration by syringe, and/or enabling use of a smaller needle for injection. Crystalline suspensions of the present invention may be delivered by subcutaneous injection using small bore needles, such as 28 G insulin syringes. The reduced volume, decreased viscosity and use of a smaller needle are all likely to decrease patient discomfort accompanying subcutaneous administration of anti-PD-1 antibodies, which is of particular concern when a drug is intended for self-administration (e.g., by prefilled syringe).

The high-concentration crystalline suspensions of anti-PD-1 antibodies of the invention also exhibit superior properties about the pharmacokinetics of drug delivery. Compared with the corresponding solution formulations, the high concentration crystalline suspensions of the invention exhibit delayed bioavailability. This time-released delivery of the anti-PD-1 antibody drug into the circulation in the subject can advantageously increase the time over which the drug is present at an effective dose for a given administration. This can reduce the initial spike in drug concentration that would otherwise occur soon after administration (e.g., subcutaneous delivery of a solution formulation), and may enable less frequent dosing.

Crystalline anti-PD-1 antibodies also have other advantageous properties. High concentration suspensions of the crystalline anti-PD-1 antibodies will likely have improved stability compared with corresponding solution formulations, i.e., the crystalline suspensions will retain anti-PD-1 biological activity for a longer time. High concentration suspensions of the crystalline anti-PD-1 antibodies can be stored at room temperature, whereas typical solution formulations require storage at 4° C. The longer shelf-life, and the ability to store the suspensions of the present invention at room temperature, offer significant advantages in handling of drug product and supply chain management.

High concentration crystalline suspensions comprising crystals of anti-PD-1 antibodies exhibiting substantially similar properties are also encompassed in certain embodiments.

The invention further provides various methods for making high concentration crystalline anti-PD-1 antibody suspensions/formulations.

Examples of methods for commercial scale production of crystalline anti-PD-1 e.g., for therapeutic use, include crystallization protocols more suited to large-scale production, such as batch crystallization and bulk dialysis crystallization. Although the specific disclosed embodiments employ a defined ratio of an antibody solution with an additive such as caffeine or other precipitant solution, any modification of the method that ends up with approximately the same concentrations of solution components in the final crystallization solution (from which crystals arise) would be equivalent. Specifically, the concentrations of the components in the caffeine or precipitant solution may be proportionally increased or decreased if the precipitant solution comprises less than or more than 50% of the final volume of the crystallization solution, respectively.

The crystallization methods of the present invention also provide a method of purifying anti-PD-1 antibodies, even if such crystals are re-dissolved prior to use. In one embodiment, an anti-PD-1 antibody is produced and at least partially purified by methods described elsewhere herein and known in the art. The antibody is then crystallized, e.g., by batch crystallization or by bulk dialysis. The crystalline antibody can be re-dissolved in 10 mM HEPES buffer pH 7.0 or any suitable buffer for the intended use of the purified antibody. For therapeutic uses, suitable pharmaceutically acceptable buffers and excipients are used. All the suggested ingredients would be GRAS (generally regarded as safe) reagents and in concentrations acceptable for formulation.

The crystallization methods of the present invention also provide a method of storing purified anti-PD-1 antibodies, even if such crystals are re-dissolved prior to use. In one embodiment, an anti-PD-1 antibody is produced and at least partially purified by methods described elsewhere herein and known in the art. The antibody is then crystallized, e.g., by batch crystallization or by bulk dialysis.

The resulting high concentration anti-PD-1 crystalline suspension could act as a stable concentrated preparation suitable for shipping and reformulating at global formulation sites.

In one embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) an additive selected from the group consisting of: caffeine, theophylline, 2′ deoxyguanosine-5′-monophosphate, a bioactive gibberellin, and a pharmaceutically acceptable salt of a bioactive gibberellin, wherein the crystallization solution has a pH of about 5.0 to about 8.0; and (b) incubating the crystallization solution for a period of time sufficient for crystal formation.

In one embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) an additive selected from the group consisting of: caffeine, theophylline, 2′ deoxyguanosine-5′-monophosphate, a bioactive gibberellin, and a pharmaceutically acceptable salt of a bioactive gibberellin, wherein the crystallization solution has a pH of about 5.0 to about 8.0; (b) concentrating the aqueous crystallization solution on a filter; and (c) incubating the crystallization solution for a period of time sufficient for crystal formation.

In another embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) an additive selected from the group consisting of: caffeine, theophylline, 2′ deoxyguanosine-5′-monophosphate, a bioactive gibberellin, and a pharmaceutically acceptable salt of a bioactive gibberellin, wherein the crystallization solution has a pH of about 5.0 to about 8.0; (b) incubating the crystallization solution for a period of time sufficient for crystal formation; and (c) optionally harvesting the crystalline anti-PD-1 mAb from the solution.

In yet a further embodiment, the anti-PD-1 mAb is concentrated prior to mixing the aqueous buffered solution.

In another embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) an additive selected from the group consisting of: caffeine, theophylline, 2′ deoxyguanosine-5′-monophosphate, a bioactive gibberellin, and a pharmaceutically acceptable salt of a bioactive gibberellin, wherein the crystallization solution has a pH of about 5.0 to about 8.0; (b) concentrating the aqueous crystallization solution on a filter; (c) incubating the crystallization solution for a period of time sufficient for crystal formation; and (d) optionally harvesting the crystalline anti-PD-1 mAb from the solution.

The resulting crystalline suspensions comprise anti-PD-1 mAb crystals, e.g., pembrolizumab crystals, having a particle size of 0.5-200 microns following harvest. In a further embodiment, the method further comprises the step of homogenizing the crystals. In still further embodiments, the crystalline anti-PD-1 mAb is harvested from the crystallization solution, or at least partially purified from the crystallization solution and the harvested or purified crystals are then homogenized. The resulting anti-PD-1 mAb crystals, e.g., pembrolizumab crystals, have a particle size following homogenization of from about 0.5 to about 50 microns.

The invention further provides various methods for making the high concentration crystalline pembrolizumab antibody solution of the invention as provided in the Examples.

In some embodiments, dextran sodium sulfate is added to the crystallization solution to allow more control over nucleation; thus, allowing growth of larger crystals. In another embodiment, dextran sodium sulfate is added to the crystallization solution to all control over the change in crystal form.

Examples provide crystallization methods suited to large-scale production, such as batch crystallization and bulk dialysis crystallization, which are useful for commercial scale production of crystalline pembrolizumab, or a pembrolizumab variant, for therapeutic use. Methods of harvesting crystals include using centrifugation or filtration (such as hollow fiber tangential flow filtration, may also be used to harvest crystals, e.g., at commercial scale).

Any modification of the disclosed methods that result in approximately the same concentrations of solution components in the final crystallization solution (from which crystals arise) would be equivalent. For example, the concentrations of the components in the precipitant solution may be proportionally increased or decreased if using a precipitant solution (a solution comprising an additive, as defined herein) that comprises less than or more than 50% of the final volume of the crystallization solution, respectively.

Embodiments of the crystallization methods of the invention also provide a process for purifying pembrolizumab or pembrolizumab variant antibodies, even if such crystals are re-dissolved prior to use. In one embodiment, a pembrolizumab antibody is produced and at least partially purified by methods described herein and known in the art. The antibody is then crystallized, e.g., by batch crystallization or by bulk dialysis. The crystalline antibody is then recovered and washed, and re-dissolved in buffer, e.g., 10 mM HEPES buffer, pH 7.0 or any suitable buffer for the intended use of the purified antibody. For therapeutic uses, suitable pharmaceutically acceptable buffers and excipients are used.

Embodiments of the crystallization methods of the invention also provide a method of storing purified pembrolizumab antibodies, even if such crystals are re-dissolved prior to use. In one embodiment, a pembrolizumab or pembrolizumab variant antibody is produced and at least partially purified by methods described herein and known in the art. The antibody is then crystallized, e.g., by batch crystallization or by bulk dialysis. The resulting concentrated pembrolizumab crystalline suspension is stored as a stable concentrated preparation suitable for shipping and reformulating at global formulation sites.

High concentration suspensions/formulations comprising crystalline pembrolizumab antibodies of the invention have several advantageous properties for use in therapy including low viscosity, which enables more efficient administration to a subject, e.g., by subcutaneous injection.

Crystalline pembrolizumab antibodies also have other advantageous properties. Suspensions of the crystalline pembrolizumab antibodies show comparable stability to the starting solution formulation and may allow for a longer shelf-life. Additionally, the ability to store the suspensions of the crystals of present invention at room temperature may offer significant advantages in handling of drug product and supply chain management.

Previous crystalline suspensions of pembrolizumab were made using a high salt process. See WO 2016/137850. Previous crystalline suspensions of pembrolizumab were made using caffeine. See WO WO2020/092233. This disclosure enables a high concentration crystalline formulation of anti-PD-1 antibodies for use in the treatment of patients with cancer that is stable with minimal aggregation and adequate viscosity. In one aspect, the invention relates to a method of producing a high concentration crystalline suspension of an anti-PD-1 monoclonal antibody comprising mixing a buffered solution comprising the mAb and an additive selected from the group consisting of caffeine, theophylline, 2′ deoxyguanosine-5′-monophosphate, a bioactive gibberellin, and a pharmaceutically acceptable salt of said bioactive gibberellin and concentrating the aqueous buffered solution to allow for a high concentration solution.

In one embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) caffeine to form a crystallization solution, wherein the crystallization solution has a pH of about 5.0 to about 8.0; (b) incubating the crystallization solution for a period of time sufficient for crystal formation; and (c) optionally harvesting the crystalline anti-PD-1 mAb from the solution. In a further embodiment, the anti-PD-1 mAb is concentrated prior to mixing in the aqueous buffered solution.

In one embodiment, the invention relates to a method for producing a high concentration crystalline anti-PD-1 mAb solution comprising: (a) mixing: (i) an aqueous buffered solution comprising about 150 mg/mL to about 300 mg/mL of the mAb, and (ii) caffeine to form a crystallization solution, wherein the crystallization solution has a pH of about 5.0 to about 8.0; (b) concentrating the aqueous crystallization solution on a filter; (c) incubating the crystallization solution for a period of time sufficient for crystal formation; and (d) optionally harvesting the crystalline anti-PD-1 mAb from the solution.

In another embodiment, the invention relates to a method for producing a high concentration crystalline anti-PD-1 mAb solution comprising: (a) mixing: (i) an aqueous buffered solution comprising about 150 mg/mL to about 200 mg/mL of the mAb, and (ii) caffeine to form a crystallization solution, wherein the crystallization solution has a pH of about 5.0 to about 8.0; (b) incubating the crystallization solution for a period of time sufficient for crystal formation; and (c) optionally harvesting the crystalline anti-PD-1 mAb from the solution. In a further embodiment, the anti-PD-1 mAb is concentrated prior to mixing in the aqueous buffered solution.

In another embodiment, the invention relates to a method for producing a high concentration crystalline anti-PD-1 mAb solution comprising: (a) mixing: (i) an aqueous buffered solution comprising about 150 mg/mL to about 200 mg/mL of the mAb, and (ii) caffeine to form a crystallization solution, wherein the crystallization solution has a pH of about 5.0 to about 8.0; (b) concentrating the aqueous crystallization solution on a filter; (c) incubating the crystallization solution for a period of time sufficient for crystal formation; and (d) optionally harvesting the crystalline anti-PD-1 mAb from the solution.

In yet another embodiment, the invention relates to a method for producing a high concentration crystalline anti-PD-1 mAb solution comprising: (a) mixing: (i) an aqueous buffered solution comprising about 165 mg/mL of the mAb, and (ii) caffeine to form a crystallization solution, wherein the crystallization solution has a pH of about 6.0; (b) incubating the crystallization solution for a period of time sufficient for crystal formation; and (c) optionally harvesting the crystalline anti-PD-1 mAb from the solution.

In yet another embodiment, the invention relates to a method for producing a high concentration crystalline anti-PD-1 mAb solution comprising: (a) mixing: (i) an aqueous buffered solution comprising about 165 mg/mL of the mAb, and (ii) caffeine to form a crystallization solution, wherein the crystallization solution has a pH of about 6.0; (b) concentrating the aqueous crystallization solution on a filter; (c) incubating the crystallization solution for a period of time sufficient for crystal formation; and (d) optionally harvesting the crystalline anti-PD-1 mAb from the solution.

In a specific embodiment, the anti-PD-1 mAb is pembrolizumab or a pembrolizumab variant. In a specific embodiment, the anti-PD-1 mAb is pembrolizumab.

In one embodiment, the anti-PD-1 mAb is concentrated prior to mixing the aqueous crystallization solution. In a further embodiment, the anti-PD-1 mAb is diafiltered in the aqueous buffered solution and/or the aqueous crystallization solution wherein the aqueous crystallization solution comprises the additive. In a further embodiment, the anti-PD-1 mAb is concentrated on a membrane. In a further embodiment, the anti-PD-1 mAb is concentrated by ultrafiltration.

In a specific embodiment, the crystallization solution comprises caffeine, PEG3350, arginine and sodium chloride.

In a specific embodiment, the crystallization solution comprises 50 mM L-Arginine HCl, 50 mM sodium chloride, 70 mg/mL sucrose, 0.2 mg/mL polysorbate 80, 10 mM L-methionine, 10 mg/mL PEG 3350, 1.4 mg/mL caffeine, 7.7 mM histidine, 20 mM HEPES, at pH 6.0.

In one embodiment, the crystallization suspension comprises crystalline phase I, crystalline phase 2, or a mixture of the two phases as characterized by SAXS and/or NMR.

In one embodiment, a pembrolizumab crystal produced by the methods herein is identified by one of the following:
(i) 181.90, 181.40, 180.36, 179,69, 137.00, 135.17, 109.28, 108.12, 66.88 and 65.35, 40.69, 27.66, 27.24, 20.75 ppm, as identified by solid-state NMR, or
(ii) 0.55, 0.71, 0.90, 1.10, 1.16, 1.20, 1.41, 1.51, 1.55, 1.58, 1.69, 1.76, 1.79, 1.86, 1.93, 2.00, 2.08, 2.17, 2.20, 2.27, 2.34, 2.40, 2.42, 2.52, 2.63, 2.70, 2.72, 2.74, 2.78, 2.81, 2.86, 2.91, 2.95 nm-1, as identified by synchrotron SAXS, or
(iii) 0.55, 0.90, 1.10, 1.41, 1.52, 1.69, 1.84, 1.93, 2.28 nm-1 as identified by laboratory SAXS, or
(iv) 182.16, 181.54, 180.59, 179,99, 137.16, 135.43, 109.36, 108.23, 66.97 and 65.57, 40.80, 27.50, 27.01, 20.85 ppm, as identified by solid-state NMR, or
(v) 0.36, 0.60, 0.71, 0.93, 1.20, 1.42, 1.45, 1.50, 1.62, 1.68, 1.79, 1.82, 1.88, 1.99, 2.02, 2.06, 2.18, 2.19, 2.30, 2.44, 2.50, 2.58, 2.72, 2.79, 2.86, 2.88, 2.92, 2.98, 3.03. 3.09 nm-1, as identified by synchrotron SAXS, or
(vi) 0.60, 0.93, 1.20, 1.44, 1.81, 2.02. 2.20 nm-1, as identified by laboratory SAXS.

In one embodiment, the invention relates to characterizing a pembrolizumab crystal identified by at least one of the following peak profiles:
(i) 181.90, 181.40, 180.36, 179,69, 137.00, 135.17, 109.28, 108.12, 66.88 and 65.35, 40.69, 27.66, 27.24, 20.75 ppm, as identified by solid-state NMR, or
(ii) 0.55, 0.71, 0.90, 1.10, 1.16, 1.20, 1.41, 1.51, 1.55, 1.58, 1.69, 1.76, 1.79, 1.86, 1.93, 2.00, 2.08, 2.17, 2.20, 2.27, 2.34, 2.40, 2.42, 2.52, 2.63, 2.70, 2.72, 2.74, 2.78, 2.81, 2.86, 2.91, 2.95 nm-1, as identified by synchrotron SAXS, or
(iii) 0.55, 0.90, 1.10, 1.41, 1.52, 1.69, 1.84, 1.93, 2.28 nm-1 as identified by laboratory SAXS.

In another embodiment, the invention relates to characterizing a pembrolizumab crystal by at least one of the following peak profiles:
(i) 0.55, 0.71, 0.90, 1.10, 1.16, 1.20, 1.41, 1.51, 1.55, 1.58, 1.69, 1.76, 1.79, 1.86, 1.93, 2.00, 2.08, 2.17, 2.20, 2.27, 2.34, 2.40, 2.42, 2.52, 2.63, 2.70, 2.72, 2.74, 2.78, 2.81, 2.86, 2.91, 2.95 nm-1, as identified by synchrotron SAXS, or
(ii) 0.55, 0.90, 1.10, 1.41, 1.52, 1.69, 1.84, 1.93, 2.28 nm-1 as identified by laboratory SAXS.

In one embodiment, the invention relates to characterizing a pembrolizumab crystal by at least one of the following peak profiles:
(i) 182.16, 181.54, 180.59, 179,99, 137.16, 135.43, 109.36, 108.23, 66.97 and 65.57, 40.80, 27.50, 27.01, 20.85 ppm, as identified by solid-state NMR, or
(ii) 0.36, 0.60, 0.71, 0.93, 1.20, 1.42, 1.45, 1.50, 1.62, 1.68, 1.79, 1.82, 1.88, 1.99, 2.02, 2.06, 2.18, 2.19, 2.30, 2.44, 2.50, 2.58, 2.72, 2.79, 2.86, 2.88, 2.92, 2.98, 3.03. 3.09 nm-1, as identified by synchrotron SAXS, or
(iii) 0.60, 0.93, 1.20, 1.44, 1.81, 2.02. 2.20 nm-1, as identified by laboratory SAXS.

In another embodiment, the invention relates to characterizing a pembrolizumab crystal by at least one of the following peak profiles:
(i) 0.36, 0.60, 0.71, 0.93, 1.20, 1.42, 1.45, 1.50, 1.62, 1.68, 1.79, 1.82, 1.88, 1.99, 2.02, 2.06, 2.18, 2.19, 2.30, 2.44, 2.50, 2.58, 2.72, 2.79, 2.86, 2.88, 2.92, 2.98, 3.03. 3.09 nm-1, as identified by synchrotron SAXS, or
(ii) 0.60, 0.93, 1.20, 1.44, 1.81, 2.02. 2.20 nm-1, as identified by laboratory SAXS.

In one embodiment, the identification of the pembrolizumab crystal is confirmed by the top 10 peaks as identified in the peak profiles. In another embodiment, the identification of the pembrolizumab crystal is confirmed by the top 5 peaks as identified in the peak profiles.

I. Definitions and Abbreviations
As used throughout the specification and appended
claims, the following abbreviations apply:

| | |
|---|---|
| CDR | Complementarity determining region |
| CHO | Chinese hamster ovary |
| CP | Cross polarizing |
| CPS | Combined positive score |
| DFS | Disease free survival |
| DS | Drug Substance |
| ELISA | Enzyme-linked immunosorbent assay |
| FR | Framework region |
| HEPES | Hydroxyethyl-piperazineethane-sulfonic acid buffer |
| HT | High throughput |
| IEX | Ion exchange |
| IHC | Immunohistochemistry or immunohistochemical |
| IV | Intravenous |
| mAb | Monoclonal antibody |
| MAS | Magic angle spinning |
| NCI | National Cancer Institute |
| NMR | Nuclear magnetic resonance |
| PBS | Phosphate buffered saline |
| PD | Progressive disease |
| PD-1 | Programmed Death 1 |
| PD-L1 | Programmed Cell Death 1 Ligand 1 |
| PD-L2 | Programmed Cell Death 1 Ligand 2 |
| PEG | Polyethylene glycol |
| PFS | Progression free survival |
| PK | Pharmacokinetic |
| PR | Partial response |
| OR | Overall response |
| OS | Overall survival |
| Q2W | One dose every two weeks |
| Q3W | One dose every three weeks |
| Q6W | One dose every six weeks |
| QD | One dose per day |
| RECIST | Response Evaluation Criteria in Solid Tumors |
| RH | Relative humidity |
| RPLC | Reversed-phase liquid chromatography |
| RPM | Revolutions per minute |
| SC | Subcutaneous |
| SD | Stable disease or standard deviation, as dictated by the context |
| SHG | Second harmonic generation |
| SONICC | Second Order Nonlinear Imaging of Chiral Crystals |
| T/C | Treated over control tumor volume ratio |
| TPS | Tumor proportion score |
| UV-TPEF | Ultraviolet Two-Photon Excited Fluorescence |
| VH | Immunoglobulin heavy chain variable region |
| VK | Immunoglobulin kappa light chain variable region |
| w/v | Weight per volume |

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” means to administer a composition of the invention to a patient to induce a positive therapeutic effect. The terms do not necessarily indicate a total elimination of all disease or disorder symptoms. “Treating” a cancer or immune condition refers to administration of a crystalline suspension or composition of the invention to a patient having an immune condition or cancerous condition, or diagnosed with or predisposed to a cancer or a pathogenic infection (e.g., viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth.

“Treatment” may include one or more of the following: inducing/increasing an antitumor immune response, stimulating an immune response to a pathogen, toxin, and/or self-antigen, stimulating an immune response to a viral infection, decreasing the number of one or more tumor markers, inhibiting the growth or survival of tumor cells, eliminating or reducing the size of one or more cancerous lesions or tumors, decreasing the level of one or more tumor markers, ameliorating, reducing the severity or duration of the cancer, prolonging the survival of a patient relative to the expected survival in a similar untreated patient.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

Positive therapeutic effects in cancer can be measured in a number of ways (See, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the term “patient” includes non-human animals including livestock animals and domestic animals including, but not limited to, cattle, horses, sheep, swine, goats, rabbits, cats, dogs, and other mammals in need of treatment. In some embodiments, the patient is an adult patient. In other embodiments, the patient is a pediatric patient. A patient “in need of treatment” is an individual diagnosed with, suspected of having, or predisposed to a disease or disorder in which a crystalline suspension or composition of the invention is intended to treat, or a patient for whom prevention of a disorder is desired.

“Antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein, i.e., human PD-1, with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 molecule, if it binds to polypeptides comprising that sequence but does not bind to proteins lacking that sequence.

The term “pharmaceutically effective amount” or “therapeutically effective amount” means an amount whereby sufficient therapeutic composition or formulation is introduced to a patient to treat a disease or condition or at least one symptom thereof. One skilled in the art recognizes that this level may vary according to the patient's characteristics such as age, weight, etc. The term “effective amount,” when used with a crystalline suspension or composition of the invention, means an amount of suspension or composition sufficient to treat a pathological condition that it was intended to treat, e.g., a cancerous condition or inflammatory disorder. An “effective amount” of a crystal or composition of the invention means an amount sufficient to elicit the response being sought in a cell, tissue, system, animal or human. In one embodiment, the effective amount is a “therapeutically effective amount” for the alleviation of the symptoms of the disease or condition being treated. When the active compound (i.e., active ingredient) is administered as the salt, references to the amount of active ingredient are to the free acid or free base form of the compound.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1, 0.2, 0.3, 0.4, 0.5, 1.0, 2.0, 3.0, 4.0, or 5.0 of the appropriate unit. In certain embodiments, “about” can mean a variation of ±0.1%, 0.5%, 1%, 2%, 3%, 4%, 5%, or 10%. In certain embodiments, the term “about” for the purposes of solid-state NMR mean±0.1 ppm. In certain embodiments, “about” can mean a variation of +0.1%, ±0.5%, ±1%, ±2%, ±3%, ±4%, ±5%, ±6%, ±7%, ±8%, ±9%, ±10% or ±11%. In specific embodiments, when referring to the dosage of “about 380 mg,” the dosage can be, for example, from 260 mg to 340 mg, from 265 mg to 335 mg, from 270 mg to 330 mg, from 275 mg to 325 mg, from 280 mg to 320 mg, from 285 mg to 315 mg, from 290 mg to 310 mg, from 300 mg to 305 mg, or from 279 to 301 mg. In alternative embodiments, the dosage can be 200 mg, 220 mg, 250 mg, 280 mg, or 300 mg. When referring to the amount of time between administrations in a therapeutic treatment regimen (i.e., amount of time between administrations of the anti-PD-1 antibody or antigen binding fragment thereof, e.g., “about 3 weeks,” which is used interchangeably herein with “approximately every three weeks” or “about 6 weeks,” which is used interchangeably herein with “approximately every six weeks”), “about” refers to the stated time+a variation that can occur due to patient/clinician scheduling and availability around the 3-week target date. For example, “about 3 weeks” can refer to 3 weeks ±5 days, 3 weeks ±4 days, 3 weeks ±3 days, 3 weeks ±2 days or 3 weeks ±1 day, or may refer to 2 weeks, 2 days through 3 weeks, 5 days.

The terms “cancer”, “cancerous”, or “malignant” refer to or describe the physiological condition in mammals that is typically characterized by unregulated cell growth. Examples of cancer include but are not limited to, melanoma, non-small cell lung cancer, small cell lung cancer, head and neck cancer, urothelial cancer, breast cancer, gastrointestinal cancer, gastroesophageal junction adenocarcinoma, multiple myeloma, hepatocellular cancer, non-Hodgkin lymphoma, primary mediastinal large B-cell lymphoma, renal cancer, Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, and salivary cancer.

“Concentration”, when used with reference to a crystalline antibody suspension of the present invention, refers to the amount of antibody (e.g., pembrolizumab) present in each macroscopic unit volume of solution. The term concentration is used in its customary sense despite the inherent heterogeneity of the suspension, as compared to a traditional solution. The concentration of antibody in a crystalline suspension is equal to the concentration of an equivalent sample in which the antibody is not in crystalline form.

“Anti-PD-1 monoclonal antibody crystal” or “crystalline anti-PD-1 mAb,” as used herein, refers to a crystal containing the antibody arranged in a lattice structure that repeats periodically in three dimensions. In contrast, a solid, amorphous form of the mAb, e.g., such as produced by lyophilizing a mAb dissolved in a solution, does not display the optical properties such as refractive index and birefringence that are typical of a crystalline antibody form.

A “high concentration” crystalline anti-PD-1 mAb solution, suspension or formulation means such a solution, suspension or formulation wherein the concentration of the anti-PD-1 mAb is at least 150 mg/mL.

“Anti-PD-1 antibody” as used in any of the treatment methods, compositions and uses herein, refers to monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, compositions and uses of the invention in which a human individual is being treated, the anti-PD-1 antibody, or antigen binding fragment thereof, is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1, IgG2, IgG3 and IgG4 constant regions, and in particular embodiments, the human constant region is an IgG1 or IgG4 constant region. In some embodiments, the antigen binding fragment is selected from the group consisting of Fab, Fab′-SH, F(ab′)₂, scFv and Fv fragments.

An “antibody solution” refers to a solution of an anti-human PD-1 antibody, e.g., pembrolizumab, which is used to generate the crystalline antibody of the present invention. “Precipitant solution” refers to a second solution that is mixed with the antibody solution, typically at a 1:1 volume ratio (i.e., equal volumes of the two solutions are mixed) to create a “crystallization solution” or “crystal solution” from which antibodies grow. The concentrations of the antibody and precipitant solutions are provided herein for a 1:1 mixture, for convenience, but one skilled in the art would recognize that the volume ratio used to make the mixture can be changed, and thus so can the concentrations of the solutions making up the mixture. Such modifications are within the scope of the invention if they generate the same crystallization conditions (i.e., the same crystallization solution) as the mixtures described herein.

With regard to crystallization methods based on dialysis, “dialysis solution” refers to the solution against which a solution of pembrolizumab (the “antibody solution”) is dialyzed to drive formation of the crystalline antibody of the present invention. “Retentate” refers to the antibody solution after dialysis, which may include crystals of the antibody, which are harvested. The antibody solution/retentate are on one side of the dialysis membrane, and the dialysis solution is on the opposite side.

The term “homogenize” means to reduce crystal particles in size using mechanical means; thus, resulting in smaller particles that are more uniform and evenly distributed. Homogenization can be performed through any known means such as using a homogenizer, or by forcing the crystalline particles through a smaller orifice (Venturi effect), such as a syringe, to break the particles into a smaller size.

The terms “micron” and “micrometer” are used interchangeably herein, and each means 1/1000000th of a meter.

“PD-L1” or “PD-L2” expression means any detectable level of expression of the designated PD-L protein on the cell surface or of the designated PD-L mRNA within a cell or tissue. PD-L protein expression may be detected with a diagnostic PD-L antibody in an immunohistochemical (IHC) assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

Several approaches have been described for quantifying PD-L1 protein expression in IHC assays of tumor tissue sections. See, e.g., Thompson, R. H., et al., Proc. Natl. Acad. Sci USA 101 (49): 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4: 127ra37 (2012); and Toplian, S. L. et al., New Eng. J Med. 366 (26): 2443-2454 (2012).

One approach employs a simple binary endpoint of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. In some embodiments, PD-L1 expression in tumor cells is counted as negative if the score is <5% score and positive if the score is >5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD-L1 antibody may also be scored for PD-L1 protein expression by assessing PD-L1 expression in both the tumor cells and infiltrating immune cells in the tissue section using a scoring process. See WO 2014/165422. One PD-L1 scoring process comprises examining each tumor nest in the tissue section for staining and assigning to the tissue section one or both of a modified H score (MHS) and a modified proportion score (MPS). To assign the MHS, four separate percentages are estimated across all the viable tumor cells and stained mononuclear inflammatory cells in all the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate, or strong staining percentages. The estimated percentages, the sum of which is 100%, are then input into the formula of 1×(percent of weak staining cells)+2×(percent of moderate staining cells)+3×(percent of strong staining cells), and the result is assigned to the tissue section as the MHS. The MPS is assigned by estimating, across all the viable tumor cells and stained mononuclear inflammatory cells in all the examined tumor nests, the percentage of cells that have at least partial membrane staining of any intensity, and the resulting percentage is assigned to the tissue section as the MPS. In some embodiments, the tumor is designated as positive for PD-L1 expression if the MHS or the MPS is positive.

The level of PD-L mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some embodiments, PD-L1 expression in a tumor sample is determined to be elevated if PD-L1 protein (and/or PD-L1 mRNA) in the sample is at least 10%, 20%, 30%, 40% or 50% greater than in the control.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Whitehouse Station, NJ USA; initial U.S. approval 2014, updated March 2021). Each light chain of pembrolizumab comprises light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 4, 5 and 6. The variable chain light (V_{L}) and heavy (V_{H}) chains of pembrolizumab comprise a sequence of amino acids as set forth in SEQ ID NO: 7 and SEQ ID NO:8, respectively and the full length light and heavy chains comprise or consist of a sequence of amino acids as set forth in SEQ ID NO:9 and SEQ ID NO:10, respectively.

As used herein, a “pembrolizumab variant” refers to a variant or derivative of a pembrolizumab antibody that (1) substantially retains its biological activity of binding to antigen (i.e., human PD-1) and inhibiting its activity (e.g., blocking the binding of PD-1 to PD-L1 and/or PD-L2) and (2) retains the ability of the antibody to bind to an additive that is used in the crystallization solution in the methods of the invention, wherein the additive is caffeine, theophylline, 2′ deoxyguanosine-5′-monophosphate, a bioactive gibberellin, such as gibberellin A3, or a pharmaceutically acceptable salt thereof. In embodiments of the invention, a pembrolizumab variant comprises light chain and heavy chain sequences that are identical to those in pembrolizumab (SEQ ID NO:9 and 10, respectively), except for having up to 10, up to 9, up to 8, up to 7, up to 6, up to 5, up to 4, up to 3, up to 2, 1, 2, 3, 4, 5, 6, 7, 8, 9, or 10 conservative amino acid substitutions at amino acid positions that are located outside of the light chain CDRs and outside of the heavy chain CDRs, e.g., the variant positions are located in the framework regions or the constant region. In further embodiments, a pembrolizumab variant has up to 10, up to 9, up to 8, up to 7, up to 6, up to 5, up to 4, up to 3, up to 2, 1, 2, 3, 4, 5, 6, 7, 8, 9, or 10 conservative amino acid substitutions that are located outside the pembrolizumab light and heavy chain CDRs and are further outside of the pembrolizumab residues that bind to caffeine, i.e., outside of TYR 436 and ASN 434 of the pembrolizumab heavy chain (positions 434 and 436 of SEQ ID NO: 10). In other words, pembrolizumab and a pembrolizumab variant comprise identical CDR sequences, but differ from each other due to having a conservative amino acid substitution at no more than ten other positions in their full-length light and heavy chain sequences, respectively. A pembrolizumab variant is substantially the same as pembrolizumab with respect to the following properties: binding affinity to PD-1, ability to block the binding of each of PD-L1 and PD-L2 to PD-1, and ability to bind to an additive selected from: caffeine, theophylline, 2′ deoxyguanosine-5′-monophosphate, a bioactive gibberellin, such as gibberellin A3, and a pharmaceutically acceptable salt of said bioactive gibberellin.

A “precipitant” is a compound that decreases the solubility of a polypeptide, such as an antibody, in a concentrated solution. In batch crystallization methods, the precipitant may be included in the “precipitant solution,” and in bulk dialysis methods the precipitant may be included in the “dialysis solution.” Precipitants induce crystallization by forming an energetically unfavorable precipitant-depleted layer around the polypeptide molecules. To minimize the relative amount of this depletion layer, the polypeptides form associations and, ultimately, crystals. This process is explained in Weber (1991) Advances in Protein Chemistry 41:1. Various precipitants are known in the art and include but are not limited to: ammonium sulfate, ammonium di-hydrogen phosphate, ethanol, isopropanol, propylene glycol, 3-ethyl-2, 4 pentane diol; and many of the polyglycols, such as polyethylene glycol (e.g., PEG 4000 and PEG 10000). In the methods of the invention, the precipitant is polyethylene glycol (e.g., PEG 3350).

In addition to precipitants, one or more additives which facilitate crystallization is added to the polypeptide precipitant solution or crystallization solution selected from: caffeine, theophylline, 2′ deoxyguanosine-5′-monophosphate, a bioactive gibberellin, and a pharmaceutically acceptable salt of the bioactive gibberellin. Two of the additives useful in the methods of the invention, caffeine, and theophylline, were found to share structural similarity as shown below:

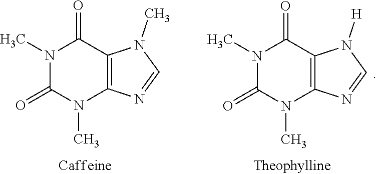

It is also shown herein that gibberellin A3 (alternatively, GA3 or gibberellic acid) is a useful reagent in the crystallization methods of the methods of the invention. Gibberellins (also known as GAs) are a class of hormones found in plants, which share a common diterpenoid acid structure and regulate various developmental processes. “Bioactive gibberellins,” are involved in different aspects of plant germination and share the following structural traits: 1) a hydroxyl group on C-3β, 2) a carboxyl group on C-6, and 3) a lactone between C-4 and C-10 (see below). Based on the similar structure and function of the “bioactive gibberellins,” which comprise gibberellin A1 (GA1), gibberellin A3 (GA3), gibberellin A4 (GA4), and gibberellin A7 (GA7), or pharmaceutically acceptable salts thereof, it is expected that any bioactive gibberellin or pharmaceutically acceptable salt thereof would be useful in the methods of the invention.

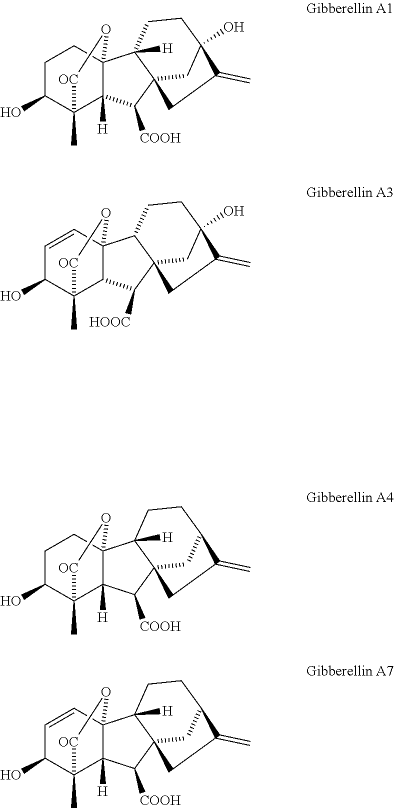

In addition to precipitants, one or more additional excipients may be added to the polypeptide precipitant solution or crystallization solution. Excipients include buffers, such as Tris or HEPES, to adjust the pH of the solution (and hence surface charge on the peptide), salts, such as sodium chloride, lithium chloride and sodium citrate, to reduce the solubility of the polypeptide.

“Tissue Section” refers to a single part or piece of a tissue sample, e.g., a thin slice of tissue cut from a sample of a normal tissue or of a tumor.

“Tris” or (2-Amino-2-hydroxymethyl-propane-1,3-diol) as used herein is synonymous with TRIS, Tris base, Trizma, Trisamine, THAM, Tromethamine, Trometamol, Tromethane, and Trisaminol.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT, or MRI scans.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all at least one, and typically two, variable domains, in which all or substantially all the hypervariable loops correspond to those of a non-human immunoglobulin and all or substantially all the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

Antibodies useful in the compositions of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions, and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

“Hypervariable region” refers to the amino acid residues of an antibody that are responsible for antigen-binding and are variable in sequence between different antibodies. The hypervariable region comprises amino acid residues from a “complementarity determining region” or “CDR” (e.g., residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain as measured by the Kabat numbering system (Kabat et al. (1991) Sequences of Proteins of Immunological Interest, 5th Ed. Public Health Service, National Institutes of Health, Bethesda, Md.) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196:901-917). As used herein, the term “framework” or “FR” residues refers to those variable domain residues other than the hypervariable region residues defined herein as CDR residues. CDR and FR residues are determined according to the standard sequence definition of Kabat. Kabat et al. (1987) Sequences of Proteins of Immunological Interest, National Institutes of Health, Bethesda Md.

“Conservatively modified variants” or “conservative substitution” refers to substitutions of amino acids are known to those of skill in this art and may be made generally without altering the biological activity of the resulting molecule, even in essential regions of the polypeptide. Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In addition, those of skill in this art recognize that, in general, single amino acid substitutions in non-essential regions of a polypeptide do not substantially alter biological activity. See, e.g., Watson et al. (1987) Molecular Biology of the Gene, The Benjamin/Cummings Pub. Co., p. 224 (4^{th} Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

The term “buffer” encompasses those agents which maintain the solution pH of the formulations of the invention in an acceptable range, or, for lyophilized formulations of the invention, provide an acceptable solution pH prior to lyophilization.

The term “pharmaceutical formulation” refers to preparations which are in such form as to permit the active ingredients to be effective, and which contains no additional components which are toxic to the subjects to which the formulation would be administered.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“Room temperature,” or “RT” as used herein refers to a temperature in the range of about 18° C. to about 25° C. (about 64 to about 77° F.).

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months.

As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), or at least about 95 wt. % (e.g., from about 95 wt. % to about 99 wt. %, or from about 98 wt. % to 100 wt. %), and most preferably at least about 99 wt. % (e.g., 100 wt. %) of a product containing a crystalline anti-PD-1 antibody, e.g., crystalline pembrolizumab or a variant thereof, or its salt (e.g., the product isolated from a reaction mixture affording the crystalline anti-PD-1 antibody or salt) consists of the crystalline anti-PD-1 antibody or salt. The level of purity of the crystalline anti-PD-1 antibody and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A crystalline anti-PD-1 antibody or salt of 100% purity is one which is free of detectable impurities as determined by a standard method of analysis.

### II. Anti-PD-1 Antibodies for Use in the Methods of the Invention

In the methods of producing anti-PD-1 mAb crystals, and the methods of use/methods of treatment of the invention the anti-human PD-1 antibody is pembrolizumab or a pembrolizumab variant. The amino acid sequences of pembrolizumab are provided in Table 2.

TABLE 2
Pembrolizumab Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 7 |
| Region | QQKPGQAPRLLIYLASYLESGVPARFSGSGSGTDFTLTISS | |
| | LEPEDFAVYYCQHSRDLPLTFGGGTKVEIK | |
| Light | EIVLTQSPATLSLSPGERATLSCRASKGVSTSGYSYLHWY | 9 |
| Chain | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 4 |
| CDR2 | GINPSNGGTNFNEKFKN | 5 |
| CDR3 | RDYRFDMGFDY | 6 |
| Variable | QVQLVQSGVEVKKPGASVKVSCKASGYTFTNYYMYWV | 8 |
| Region | RQAPGQGLEWMGGINPSNGGTNFNEKFKNRVTLTTDSS | |
| | TTTAYMELKSLQFDDTAVYYCARRDYRFDMGFDYWGQ | |
| | GTTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSS | |
| | TTTAYMELKSLQFDDTAVYYCARRDYRFDMGFDYWGQ | |
| | GTTVTVSSASTKGPSVFPLAPCSRSTSESTAALGCLVKDY | |
| | FPEPVTVSWNSGALTSGVHTFPAVLQSSGLYSLSSVVTVP | |
| | SSSLGTKTYTCNVDHKPSNTKVDKRVESKYGPPCPPCPA | |
| | PEFLGGPSVFLFPPKPKDTLMISRTPEVTCVVVDVSQEDP | |
| | EVQFNWYVDGVEVHNAKTKPREEQFNSTYRVVSVLTVL | |
| | HQDWLNGKEYKCKVSNKGLPSSIEKTISKAKGQPREPQV | |
| | YTLPPSQEEMTKNQVSLTCLVKGFYPSDIAVEWESNGQP | |
| | ENNYKTTPPVLDSDGSFFLYSRLTVDKSRWQEGNVFSCS | |
| | VMHEALHNHYTQKSLSLSLGK | |

The crystalline anti-PD-1 mAbs of the invention comprise three light chain CDRs (CDRL1, CDRL2 and CDRL3) and three heavy chain CDRs (CDRH1, CDRH2 and CDRH3). In one embodiment, the three light chain CDRs are SEQ ID NO:1, SEQ ID NO:2, and SEQ ID NO:3 and the three heavy chain CDRs are SEQ ID NO:4, SEQ ID NO:5 and SEQ ID NO:6.

In certain embodiments, the invention provides a crystalline anti-PD-1 mAb comprising a light chain variable region (V_{L}) comprising SEQ ID NO:7 or a variant of SEQ ID NO: 7 and a heavy chain variable region (V_{H}) comprising SEQ ID NO:8 or a variant of SEQ ID NO:8. In some embodiments, a variant light chain or heavy chain variable region sequence is identical to the reference sequence except having one, two, three, four, five, six, seven, eight, nine or ten amino acid substitutions. In particular embodiments, the amino acid substitutions are conservative amino acid substitutions. The substitutions in the pembrolizumab variants are in the framework region (i.e., outside of the CDRs) or the constant region and are outside of any residues that would inhibit binding of the pembrolizumab variant to the additive used in the methods herein and thus inhibit crystallization.

In one embodiment of the invention, the crystalline anti-human PD-1 antibody comprises a light chain variable region (V_{L}) comprising or consisting of SEQ ID NO:7 and a heavy chain variable region (V_{H}) comprising or consisting of SEQ ID NO:8.

In another embodiment, the crystalline anti-PD-1 mAb of the invention comprises a V_{L} domain and/or a V_{H} domain with at least 99%, 98%, 97%, 96%, 95%, 94%, 93%, 92%, 91% or 90%, sequence homology to the V_{L} domain or V_{H} domain described above and exhibits specific binding to PD-1. In another embodiment, the crystalline anti-PD-1 mAb comprises V_{L} and V_{H} domains having up to 1, 2, 3, 4, 5, 6, 7, 8, 9, or 10 or more amino acid substitutions, and exhibits specific binding to PD-1.

In any of the embodiments above, the anti-PD-1 crystals of the invention may comprise a full-length anti-PD-1 antibody (e.g., pembrolizumab) or may be an antigen binding fragment comprising a short truncation that (1) comprises the light chain CDRs of SEQ ID NO:1, SEQ ID NO:2, and SEQ ID NO:3 and the heavy chain CDRs of SEQ ID NO: 4, SEQ ID NO:5 and SEQ ID NO:6, (2) specifically binds human PD-1 and (3) specifically binds to the additive used in the methods of the invention. In certain embodiments, the anti-PD-1 antibody is a full-length anti-PD-1 antibody selected from any class of immunoglobulins, including IgM, IgG, IgD, IgA, and IgE. Preferably, the antibody is an IgG antibody. Any isotype of IgG can be used, including IgG1, IgG2, IgG₃, and IgG4. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the crystalline anti-PD-1 mAb is an anti-PD-1 antibody comprising a light chain comprising or consisting of a sequence of amino acid residues as set forth in SEQ ID NO:9 and a heavy chain comprising or consisting of a sequence of amino acid residues as set forth in SEQ ID NO: 10. In some embodiments of the invention, the crystalline anti-PD-1 mAb of the invention is crystalline pembrolizumab or a pembrolizumab biosimilar.

In further embodiments, the crystalline anti-PD-1 mAb is a pembrolizumab variant having up to 10, up to 9, up to 8, up to 7, up to 6, up to 5, up to 4, up to 3, up to 2, 1, 2, 3, 4, 5, 6, 7, 8, 9, or 10 conservative amino acid substitutions that are located outside the pembrolizumab light and heavy chain CDRs and are further outside of the pembrolizumab residues that bind to caffeine, i.e., outside of TYR 436 and ASN 434 of the pembrolizumab heavy chain (positions 434 and 436 of SEQ ID NO: 10).

Ordinarily, amino acid sequence variants of the crystalline pembrolizumab variants of the invention will have an amino acid sequence having at least 90% amino acid sequence identity with the amino acid sequence of the reference antibody (e.g., heavy chain, light chain, V_{H}, or V_{L} sequence), more preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### III. Methods of Producing Crystalline Antibody Suspensions

In one aspect, the invention relates to high concentration crystalline suspensions of anti-PD-1 monoclonal antibody obtained by lyo cake, dialysis, and high concentrated liquid preparations. Conditions were established to prepare crystalline suspensions by bulk crystallization methods (batch, dialysis, and lyo cake) in high yield.

In one aspect, the invention relates to a method of producing a crystalline anti-PD-1 monoclonal antibody (mAb) comprising:
a. mixing
   i. an aqueous buffered solution comprising about 150 mg/mL to 300 mg/mL of the mAb, wherein the anti-PD-1 mAb is pembrolizumab or a pembrolizumab variant, with a precipitant solution to form a crystallization solution,
   ii. an additive selected from the group consisting of caffeine, theophylline, 2′ deoxyguanosine-5′-monophosphate, a bioactive gibberellin, and a pharmaceutically acceptable salt of said bioactive gibberellin,
      to form a crystallization solution, wherein the crystallization solution has a pH of about 5.0 to 8.0
b. concentrating the aqueous buffered solution on a filter; and
c. incubating the crystallization solution for a time sufficient for crystal formation (crystallization).

In one aspect, the invention relates to a method of producing a crystalline anti-PD-1 monoclonal antibody (mAb) comprising:
a. mixing
   i. an aqueous buffered solution comprising about 150 mg/mL to 300 mg/mL of the mAb, wherein the anti-PD-1 mAb is pembrolizumab or a pembrolizumab variant, with a precipitant solution to form a crystallization solution,
   ii. an additive selected from the group consisting of caffeine, theophylline, 2′ deoxyguanosine-5′-monophosphate, a bioactive gibberellin, and a pharmaceutically acceptable salt of said bioactive gibberellin,
b. concentrating the aqueous buffered solution on a filter;
c. incubating the crystallization solution for a time sufficient for crystal formation (crystallization), and
d. harvesting the crystals from the solution.

In one aspect, the invention relates to a method of producing a crystalline anti-PD-1 monoclonal antibody (mAb) comprising:
a. mixing
   i. an aqueous buffered solution comprising about 150 mg/mL to 300 mg/mL of the mAb, wherein the anti-PD-1 mAb is pembrolizumab or a pembrolizumab variant, with a precipitant solution to form a crystallization solution,
   ii. an additive selected from the group consisting of caffeine, theophylline, 2′ deoxyguanosine-5′-monophosphate, a bioactive gibberellin, and a pharmaceutically acceptable salt of said bioactive gibberellin, and
   iii. polyethylene glycol (PEG);
b. concentrating the aqueous buffered solution on a filter;
c. incubating the crystallization solution for a time sufficient for crystal formation (crystallization), and
d. harvesting the crystals from the solution.

In one aspect, the invention relates to a method of producing a crystalline anti-PD-1 monoclonal antibody (mAb) comprising (a) mixing an aqueous buffered solution comprising about 150 mg/ml to about 300 mg/ml of the mAb, wherein the anti-PD-1 mAb is pembrolizumab or a pembrolizumab variant, and an additive, wherein the additive is caffeine, to form a crystallization solution, wherein the crystallization solution has a pH of about 5.0 to about 8.0; b) concentrating the aqueous crystallization solution on a filter; and c) incubating the crystallization solution for a period of time sufficient to form a high concentration crystalline suspension.

In some embodiments, the precipitant solution comprises a buffer with a pH between 5 and 8, such as 4.5, 5.5, 7.5, or any other suitable value. In various embodiments, the buffers are histidine, lysine, arginine, or HEPES. In one embodiment, the buffer is histidine.

In some embodiments, the precipitant solution comprises a histidine, arginine, lysine, or HEPES buffer, wherein the buffer is at a concentration of from about 5 mM to about 100 mM. In a further embodiment, the precipitant solution comprises about 20 mM histidine, arginine, lysine, or HEPES buffer. In a further embodiment, the precipitant solution comprises about 100 mM histidine, arginine, lysine, or HEPES buffer. In a specific embodiment, the precipitant solution comprises about 5 mM to about 100 mM HEPES buffer. In a further embodiment, the precipitant solution comprises about 20 mM HEPES buffer.

In some embodiments, the method of producing a high concentration crystalline suspension additionally includes mixing PEG with the aqueous buffered solution and additive to form a crystallization solution. In another embodiment, the additive and the PEG are mixed together to form a precipitant solution before being mixed with the aqueous buffered solution comprising the mAb. In a separate embodiment, the aqueous buffered solution comprising the mAb is mixed with the additive before being mixed with PEG. In yet another embodiment, the aqueous buffered solution comprising the mAb is mixed with PEG before being mixed with the additive.

In some embodiments, the crystallization solution comprises PEG. In a further embodiment, the crystallization solution comprises PEG3350. In one embodiment, the crystallization solution comprises 0-30% PEG 3350. In another embodiment, the crystallization solution comprises 0-20% PEG 3350. In yet a further embodiment, the crystallization solution comprises 5-15% PEG 3350. In yet another embodiment, the concentration of PEG3350 is 10 mg/ml. In some embodiments, the crystallization solution comprises 0-10% PEG 3350 and 0.1-0.3% caffeine or theophylline, 2′deoxyguanosine-5-monophospahate or Gibberellin A3 or any other suitable value.

In some embodiments the incubation is performed between 4-40° C., e.g., at room temperature (e.g., 22° C.), for 1 hour, 1 day, 5 days or 10 days, or any other time sufficient to allow crystal formation. In other embodiments, the temperature is ramped-up during the incubating step, e.g., from 4° C. up to 22-40° C.

In some embodiments, the crystallization solution is seeded with crystals during the incubating step.

In some embodiments, the crystallization solution is filtered and washed with HEPES or TRIS.

In embodiments of the methods of the invention, the solution concentration of the anti-PD-1 mAb in the crystallization solution is from about 150-300 mg/mL. In further embodiments, the solution concentration of the anti-PD-1 mAb in the crystallization solution is from about 170 mg/mL to about 300 mg/mL, about 200 mg/mL to about 280 mg/mL, about 200 mg/mL to about 220 mg/mL. In specific embodiments of the invention, the solution concentration of the anti-PD-1 mAb in the crystallization solution is about 250 mg/mL. In specific embodiments of the invention, the solution concentration of the anti-PD-1 mAb in the crystallization solution is about 225 mg/mL. In specific embodiments of the invention, the solution concentration of the anti-PD-1 mAb in the crystallization solution is about 165 mg/mL.

In embodiments of the methods of the invention, the solution concentration of the anti-PD-1 mAb in the crystallization solution is from about 150-500 mg/mL. In further embodiments, the solution concentration of the anti-PD-1 mAb in the crystallization solution is from about 170 mg/mL to about 500 mg/mL, about 200 mg/mL to about 480 mg/mL, about 200 mg/mL to about 420 mg/mL, about 220 mg/mL to about 460 mg/mL, about 240 mg/mL to about 440 mg/mL, about 260 mg/mL to about 420 mg/mL, about 280 mg/mL to about 400 mg/mL, about 300 mg/mL to about 380 mg/mL, about 320 mg/mL to about 360 mg/mL, about 340 mg/ml.

In specific embodiments, the solution concentration of the anti-PD-1 mAb in the crystallization solution is about 250 mg/mL. In specific embodiments of the invention, the solution concentration of the anti-PD-1 mAb in the crystallization solution is about 225 mg/mL. In specific embodiments, the solution concentration of the anti-PD-1 mAb in the crystallization solution is about 165 mg/mL. In specific embodiments, the solution concentration of the anti-PD-1 mAb is about 130 mg/mL.

In specific embodiments, the patient is administered 320-420 mg of the anti-PD-1 antibody. In one embodiment, the patient is administered 360 mg of the anti-PD-1 antibody or antigen binding fragment thereof. In one embodiment, the patient is administered 370 of the anti-PD-1 antibody or antigen binding fragment thereof. In one embodiment, the patient is administered 375 mg of the anti-PD-1 antibody or antigen binding fragment thereof. In one embodiment, the patient is administered 380 mg of the anti-PD-1 antibody or antigen binding fragment thereof. In one embodiment, the patient is administered 385 mg of the anti-PD-1 antibody or antigen binding fragment thereof. In one embodiment, the patient is administered 385 mg of the anti-PD-1 antibody or antigen binding fragment thereof. In one embodiment, the patient is administered 390 mg of the anti-PD-1 antibody or antigen binding fragment thereof. In one embodiment, the patient is administered 395 mg of the anti-PD-1 antibody or antigen binding fragment thereof. In one embodiment, the patient is administered 400 mg of the anti-PD-1 antibody or antigen binding fragment thereof. In one embodiment, the patient is administered 420 mg of the anti-PD-1 antibody or antigen binding fragment thereof.

In some embodiments, the method comprises the step of harvesting the crystalline anti-PD-1 mAb from the solution. Methods of harvesting the crystals are known to one of skill in the art and include centrifugation, decantation, lyophilization and filtration, such as hollow fiber tangential flow filtration.

In some embodiments, the method further comprises the step of homogenizing the anti-PD-1 mAb crystals after they are harvested from the crystallization solution. The step of homogenization provides anti-PD-1 mAb crystals with a smaller particle size, e.g., 0.5 to 50 microns. Such smaller particle crystals can be used, for example, in high concentration pharmaceutical formulations.

In some embodiments, the method further comprises the step of homogenizing the anti-PD-1 mAb crystals without first harvesting said crystals from the crystallization solution. In this method, the crystallization solution can be homogenized after incubation for a sufficient time for crystal formation, e.g., forced through a syringe, without first harvesting. The smaller size anti-PD-1 mAb crystals can optionally be harvested following homogenization.

In specific embodiments, the additive and the aqueous buffered solution comprising the mAb are mixed to form a crystallization solution.

In specific embodiments, the additive and PEG are mixed to form a precipitant solution before being mixed with the aqueous buffered solution comprising the mAb. The precipitant solution and the aqueous buffered solution comprising the mAb are then mixed to form a crystallization solution.

In alternative embodiments, PEG and the additive are mixed to form a precipitant solution before being mixed with the aqueous buffered solution comprising the mAb. The precipitant solution and the aqueous buffered solution comprising the mAb are then mixed to form a crystallization solution.

In some embodiments, caffeine is mixed into the aqueous buffered solution comprising the mAb to form an aqueous solution. In some embodiments of the invention, the aqueous buffered solution is mixed into the caffeine comprising the mAb to form an aqueous solution.

In other embodiments, the aqueous buffered solution comprising the mAb is mixed with the additive to form an aqueous buffered solution comprising mAb and additive. This solution is then mixed with PEG, either as a solid or a solution.

In any of the above embodiments, the additive is caffeine, theophylline, 2′ deoxyguanosine-5′-monophosphate, a bioactive gibberellin, or a pharmaceutically acceptable salt of the gibberellin. In a specific embodiment, the additive is caffeine.

In any of the above embodiments, PEG is added to solutions having a pH of 5.5-8. In a specific embodiment, pH of the crystallization solution is less than 6.0 and the amount of PEG is about 2 to 6% w/v. In a separate embodiment, the pH of the crystallization solution is about 6.0 and the amount of PEG is about 0 to 4% w/v. In a separate embodiment, the pH of the crystallization solution is about 6.2 and the amount of PEG is 0-4% w/v. In a separate embodiment, the pH of the crystallization solution is about 6.4 and the amount of PEG is 0-4% w/v. In a separate embodiment, the pH of the crystallization solution is from about 6.8 to 8.0 and the amount of PEG is 0-2% w/v.

In any of the above embodiments, PEG does not need to be added if the pH of the crystallization solution is greater than 6.0.

In particular embodiments of the methods of the invention, the pH of the crystallization solution and the amount of PEG present in the solution is selected from the group consisting of:
pH of the crystallization solution is from about 5 to 8.4 and the amount of PEG is about 0% to about 12% w/v,
pH of the crystallization solution is about 6.4 and the amount of PEG is about 0% to about 4% w/v,
pH of the crystallization solution is from about 5 to 8.4 and the amount of PEG is about 2% to about 12% w/v, and
pH of the crystallization solution is about 8.8 and the amount of PEG is about 10% to about 12% w/v.

In certain embodiments of any of the methods above, the PEG is PEG 3350.

In one embodiment, the additive is caffeine.

In another embodiment, the additive is theophylline.

In yet another embodiment, the additive is 2′ deoxyguanosine-5′-monophosphate.

In a further embodiment, the additive is a bioactive gibberellin or a pharmaceutically acceptable salt thereof. In specific embodiments, the bioactive gibberellin is gibberellin A1, a pharmaceutically acceptable salt of gibberellin A1, gibberellin A3, a pharmaceutically acceptable salt of gibberellin A3, gibberellin A4, a pharmaceutically acceptable salt of gibberellin A4, gibberellin A7, or a pharmaceutically acceptable salt of gibberellin A7.

In particular embodiments, the additive is gibberellin A3 or a pharmaceutically acceptable salt thereof. In some embodiments, the additive is gibberellin A3. In other embodiments, the additive is a sodium salt of gibberellin A3. In other embodiments, the additive is a potassium salt of gibberellin A3. In other embodiments, the additive is an ammonium salt of gibberellin A3.

The amount of additive in the final crystallization solution is from about 0.10% to about 0.30% w/v. In other embodiments, the amount of additive is from about 0.15% to about 0.30% w/v, from about 0.175% to about 0.30% w/v, from about 0.20% to about 0.30% w/v, from about 0.225% to about 0.30% w/v, from about 0.25% to about 0.30% w/v, from about 0.10% to about 0.25% w/v, from about 0.10% to about 0.275% w/v, from about 0.10% to about 0.25% w/v, from about 0.10% to about 0.225% w/v or from about 0.10% to about 0.20% w/v. In further embodiments, the amount of additive is about 0.10% w/v, about 0.125% w/v, about 0.15% w/v, about 0.175% w/v, about 0.20% w/v, about 0.225% w/v, about 0.25% w/v, about 0.275% w/v, or about 0.30% w/v.

In one embodiment, the additive is caffeine, which is present in the final crystallization solution in an amount of about 0.15% w/v to about 0.30% w/v.

In another embodiment, the additive is theophylline, which is present in the final crystallization solution in an amount of about 0.25% w/v to about 0.30% w/v.

In any of the above embodiments, the crystallization solution further comprise about 1% to about 10% w/v dextran sodium sulfate, which slows the rate of nucleation and allows the growth of larger crystals. In another embodiment, the crystallization solution further comprises about 5% w/v dextran sodium sulfate. In certain cases, it may be desirable to make larger crystals, for example, for use in characterization studies such as x-ray crystallography. In further embodiments, the crystallization solution comprises about 1%, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% v, about 9% w/v, about 9.5% w/v, or about 10% w/v dextran sodium sulfate. In alternative embodiments the crystallization solution comprises about 1% to about 9% w/v, about 1% to about 8% w/v, about 1% to about 7% w/v, about 1% to about 6% w/v, about 1% to about 5% w/v, about 1% to about 4% w/v, about 1% to about 3% w/v, about 1% to about 2% w/v, about 2% to about 10% w/v, about 2% to about 9% w/v, about 2% to about 8% w/v, about 2% to about 7% w/v, about 2% to about 6% w/v, about 2% to about 5% w/v, about 2% to about 4%, about 2% to about 3%, about 3% to about 10%, about 3% to about 9%, about 3% to about 8% w/v, about 3% to about 7% w/v, about 3% to about 6% w/v, about 3% to about 5% w/v, about 3% to about 4% w/v, about 4% to about 10% w/v, about 4% to about 9% w/v, about 4% to about 8% w/v, about 4% to about 7% w/v, about 4% to about 6% w/v, about 4% to about 5% w/v, about 5% to about 10% w/v, about 5% to about 9% w/v, about 5% to about 8% w/v, about 5% to about 7% w/v, about 5% to about 6% w/v, about 6% to about 10% w/v, about 6% to about 9% w/v, about 6% to about 8% w/v, about 6% to about 7% w/v, about 7% to about 10% w/v, about 7% to about 9% w/v, about 7% to about 8% w/v, about 8% to about 10% w/v, about 8% to about 9% w/v, or about 9% to about 10% w/v dextran sodium sulfate.

In specific embodiments, the crystallization solution comprises about 0% to about 40% w/v PEG. The average molecular weight of the PEG is from about 2,500 to about 35,000. In one embodiment, the molecular weight of PEG is from about 2,500 to about 20,000. In particular embodiments, the PEG is PEG 3,350. In alternate embodiments, the PEG is PEG 2,500 (i.e., has an average mol. wt. of 2500), PEG 3,000, PEG 4,000, PEG 5,000, PEG 6,000, PEG 7,000, PEG 8,000, PEG 9,000, PEG 10,000, PEG 12,000, PEG 14000, PEG 15,000, PEG 1600, PEG 1800, PEG 20,000, PEG 22,000, PEG 24,000, PEG 25,000, PEG 26,000, PEG 28,000, PEG 30,000, PEG 32,000, PEG 34,000, or PEG 35,000.

In some embodiments, the amount of PEG in the crystallization solution is 0% (e.g., when the pH of the crystallization solution is greater than 6.0). In other embodiments, the amount of PEG in the crystallization solution is from about 2% to about 40% w/v; however, one skilled in the art will realize that use of different molecular weight PEGs for the methods of the invention alters the amount of PEG. In some embodiments, the PEG can be present in the crystallization solution in an amount of about 5% to about 15% w/v. In alternative embodiments, the PEG can be present in the crystallization solution in an amount of about 10% to about 30% w/v. In further embodiments, the PEG can be present in the crystallization solution in an amount of about 5% to about 35% w/v, about 5% to about 30% w/v, about 5% to about 25% w/v, about 5% to about 10% w/v, about 10% to about 40% w/v, about 5% to about 35% w/v, about 10% to about 30% w/v, about 10% to about 25% w/v, about 10% to about 20% w/v, about 10% to about 15% w/v, about 15% to about 40% w/v, about 15% to about 35% w/v, about 15% to about 30% w/v, about 15% to about 25% w/v, about 15% to about 20% w/v, about 20% to about 40% w/v, about 20% to about 35% w/v, about 20% to about 30% w/v, about 20% to about 25% w/v, about 25% to about 40% w/v, about 25% to about 35% w/v, about 25% to about 30% w/v, about 30% to about 40% w/v, or about 30% to about 35% w/v.

In the methods of the invention, the crystallization solution is made by combining: (1) an aqueous buffered solution comprising an anti-PD-1 mAb (i.e., pembrolizumab or a pembrolizumab variant), and an additive, as described herein; wherein the components of the crystallization solution can be added in any order.

In the methods of the invention, the crystallization solution is made by combining: (1) an aqueous buffered solution comprising an anti-PD-1 mAb (i.e., pembrolizumab or a pembrolizumab variant), (2) optionally, PEG, and (3) an additive, as described herein; wherein the components of the crystallization solution can be added in any order.

In embodiments of the invention, the aqueous buffered solution comprising the anti-PD-1 mAb has a pH of about 6.0 to about 8.8. In further embodiments, the pH is about 6.0, about 6.2, about 6.4, about 6.6, about 6.8, about 7.0, about 7.2, about 7.4, about 7.6, about 7.8, about 8.0, about 8.2, about 8.4, about 8.6, or about 8.8. In further embodiments, the pH of the aqueous buffered solution comprising the anti-PD-1 mAb is from about 5.0 to about 6.0. In additional embodiments, the pH is from about 6.8 to about 8.4.

In still further embodiments, the pH of the aqueous buffered solution comprising the anti-PD-1 mAb is from about 6.2 to about 7.8, from about 6.2 to about 7.6, from about 6.2 to about 7.4, from about 6.2 to about 7.2, from about 6.2 to about 7.0, from about 6.2 to about 6.8, from about 6.2 to about 6.6, from about 6.2 to about 6.4, from about 6.4 to about 7.8, from about 6.4 to about 7.6, from about 6.4 to about 7.4, from about 6.4 to about 7.2, from about 6.4 to about 7.0, from about 6.4 to about 6.8, from about 6.4 to about 6.6, from about 6.6 to about 7.8, from about 6.6 to about 7.6, from about 6.6 to about 7.4, from about 6.6 to about 7.2, from about 6.6 to about 7.0, from about 6.6 to about 6.8, from about 6.8 to about 7.8, from about 6.8 to about 7.6, from about 6.8 to about 7.4, from about 6.8 to about 7.2, from about 6.8 to about 7.0, from about 7.0 to about 7.8, from about 7.0 to about 7.6, from about 7.0 to about 7.4, from about 7.0 to about 7.2, from about 7.2 to about 7.8, from about 7.2 to about 7.6, from about 7.2 to about 7.4, from about 7.4 to about 7.8, from about 7.4 to about 7.6, from about 7.6 to about 7.8.

In one embodiment of any of the methods herein, the aqueous buffered solution comprising the mAb further comprises histidine buffer at a pH of about 5.0 to about 7.0. In specific embodiments of any of the methods herein, the aqueous buffered solution comprising the mAb further comprises histidine buffer at a pH of about 5.0 to about 6.0. In specific embodiments, the aqueous buffered solution comprising the mAb further comprises 20 mM histidine buffer at pH 5.4.

In particular embodiments of any of the methods of the invention, the crystallization solution further comprises from about 25 mM to about 250 mM HEPES buffer. In some embodiments, the crystallization solution further comprises about 25 mM, about 30 mM. about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 125 mM, about 130 mM, about 140 mM, about 150 mM, about 160 mM, about 170 mM, about 175 mM, about 180 mM, about 190 mM, about 200 mM, about 210 mM, about 220 mM, about 225 mM, about 230 mM, about 240 mM, about 245 mM, or about 250 mM HEPES buffer.

In other embodiments of the methods of the invention, the crystallization solution further comprises Tris buffer (i.e., instead of HEPES buffer) in any of the amounts specified above. In alternative embodiments, the crystallization solution further comprises PIPES, MOPS, TES, DIPSO, MOBS, or TAPSO buffer.

Following mixture of the aqueous buffered solution comprising the anti-PD-1 mAb, the crystallization solution is incubated at a temperature of from about 2° C. to about 37° C. for a length of time sufficient for crystal formation. In certain embodiments, the incubation temperature of the crystallization solution is from about 18° C. to about 25° C. In still other embodiments, the incubation temperature of the crystallization solution is from about 2° C. to about 35° C., about 2° C. to about 30° C., about 2° C. to about 25° C., about 2° C. to about 20° C., about 2° C. to about 15° C., about 2° C. to about 10° C., about 5° C. to about 37° C., about 5° C. to about 35° C., about 5° C. to about 30° C., about 5° C. to about 25° C., about 5° C. to about 20° C., about 5° C. to about 15° C., about 5° C. to about 10° C., about 10° C. to about 37° C., about 10° C. to about 35° C., about 10° C. to about 30° C., about 10° C. to about 25° C., about 10° C. to about 20° C., about 10° C. to about 15° C., about 15° C. to about 37° C., about 15° C. to about 35° C., about 15° C. to about 30° C., about 15° C. to about 25° C., about 15° C. to about 20° C., about 20° C. to about 37° C., about 20° C. to about 35° C., about 20° C. to about 30° C., about 20° C. to about 25° C., about 25° C. to about 37° C., about 25° C. to about 35° C., about 25° C. to about 30° C., about 30° C. to about 37° C., or about 30° C. to about 35° C.

In further embodiments, the crystallization solution is heated to about 50° C. where it remains in solution, and then cooled, where it only crystallizes upon cooling to a temperature of about 37° C. or lower.

In still further embodiments, the crystallization solution is heated to about 50° C., then cooled to a temperature of about 18° C. to about 25° C. or cooled to a temperature of about 25° C. or lower.

In additional embodiments, the crystallization solution is heated to about 50° C., then cooled to a temperature of about 4° C.

In particular embodiments of the method of the invention, the incubation temperature is ramped from about 4° C. to about 40° C.

In one embodiment, the crystallization solution is incubated at an incubation temperature of from about 2° C. to about 40° C. In one embodiment, the crystallization solution is incubated at an incubation temperature of from about 18° C. to about 25° C. In one embodiment, the crystallization solution is heated to about 50° C., then post-crystallization cooled to a temperature of about 37° C. or lower. In one embodiment, the crystallization solution is cooled to a temperature of about 18° C. to about 25° C. In one embodiment, the crystallization solution is cooled to a temperature of about 4° C. In one embodiment, the incubation temperature is ramped from about 4° C. to about 10-40° C. In one embodiment, the crystallization solution is incubated for about 15 minutes or more. In one embodiment, the crystallization solution is incubated for about 2 hours or more.

In any of the methods herein, the crystallization solution is incubated for a period of time sufficient for crystal formation. Crystal formation can be detected, for example, by visual inspection, or by use of SONICC™ imaging. In particular embodiments, the crystallization solution is incubated for about 15 minutes or more. In some embodiments, the crystallization solution is incubated for about 2 hours or more. In some embodiments, the crystallization solution is incubated overnight. In some embodiments, the crystallization solution is incubated 18 hours or more. In particular embodiments, the crystallization solution is incubated for about 30 minutes or more, about 1 hour or more, about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more, about 14 hours or more, about 15 hours or more, about 16 hours or more, about 17 hours or more, about 20 hours or more, or about 24 hours or more. In additional embodiments, the crystallization solution is incubated for about 2 days, 3 days, 4 days, 5 days, 1 week, 10 days, 2 weeks, 15 days, 3 weeks, or more than 3 weeks.

In particular embodiments of any of the methods described herein, the crystallization solution is rotated or agitated during incubation.

Various methods of protein crystallization are known. Giege et al. (1994) Acta Crystallogr. D50:339; McPherson (1990); Eur. J. Biochem. 189:1. Such techniques include hanging drop vapor diffusion (McPherson (1976) J. Biol. Chem. 251:6300), sitting drop vapor diffusion, microbatch and dialysis. Vapor diffusion is useful for screening to determine crystallization conditions. Such methods are also suitable for generation of large crystals for use in X-ray diffraction studies, e.g., to determine the three-dimensional structure of the anti-PD-1 antibody.

Both hanging drop and sitting drop vapor diffusion entail a droplet containing purified protein, buffer, and precipitant being allowed to equilibrate with a larger reservoir containing similar buffers and precipitants in higher concentrations. Initially, the droplet of protein solution contains an insufficient concentration of precipitant for crystallization, but as water vaporizes from the drop and transfers to the reservoir, the precipitant concentration increases to a level optimal for crystallization. Since the system is in equilibrium, these optimum conditions are maintained until the crystallization is complete. The hanging drop method differs from the sitting drop method in the vertical orientation of the protein solution drop within the system.

In the microbatch method, polypeptide is mixed with precipitants to achieve supersaturation, and the vessel is sealed and set aside until crystals appear.

In the dialysis method, polypeptide is retained on one side of a dialysis membrane which is placed into contact with a solution containing precipitant. Equilibration across the membrane increases the precipitant concentration thereby causing the polypeptide to reach supersaturation levels. For the methods of the present invention, it is desirable to use an anti-PD-1 antibody at an initial protein concentration

Uniform crystalline suspensions of therapeutic proteins present opportunities for novel drug delivery systems. Crystalline insulin suspensions have been used as sustained release preparations for over fifty years (Brange et al., 1999). Crystalline suspensions have also been proposed for the delivery of interferon (Reichert et al., 1999) and monoclonal antibodies (Yang et al., 2003). Crystalline forms also enable non-injectable delivery systems such as pulmonary delivery for local or systemic delivery of protein therapeutics (Reichert et al., 1999).

Henry Bence Jones was the first to describe naturally occurring crystals of immunoglobulin light chains, the so-called “Bence-Jones protein,” isolated the urine of a myeloma patient. Jones (1848) Phil. Trans. Royal Soc. (London) 55-62. Full-length, intact antibodies, however, are difficult to crystallize, likely due to the flexibility of their multiple (four) polypeptide chains. Although, there have been numerous reports of crystallization of intact antibodies over the last 30 years, only four structures have been deposited in the Research Collaboratory for Structural Bioinformatics Protein Databank (RCSB-PDB). In contrast, there have been over 800 structures deposited for antibody fragments, such as Fab fragments (either apo or complexed).

Some full-length therapeutic antibodies have been crystallized. Determination of the crystal structure for an anti-human IL-13 antibody is described at Int'l Pat. App. Pub. WO 2005/121177 (issued as U.S. Pat. No. 7,615,213) to Wyeth, and methods for the preparation of crystalline anti-human TNF-α crystals are described at Int'l Pat. App. Pub. WO 2008/057240 (issued as U.S. Pat. No. 8,034,906) to Abbott Biotechnology. Ltd. Methods for the preparation of crystalline anti-human IL-12 antibodies are described at Int'l Pat. App. Pub. WO 2008/121301 (issued as U.S. Pat. No. 8,168,760) to Abbott Laboratories.

Protein crystals, including antibodies, are also being developed as therapeutic compositions. Altus Pharmaceuticals, now a part of Althea Technologies, Inc., has put three crystalline protein formulations into human clinical trials: ALTU-238 (long-acting injectable formulation of somatropin); ALTU-237 (oral oxalate-degrading enzyme); and ALTU-236 (oral phenylalanine degrading enzyme). Researchers from Altus also crystallized three commercially available monoclonal antibodies (rituximab, trastuzumab and infliximab) by vapor diffusion methods or batch crystallization. Yang et al. (2003) Proc. Nat'l Acad. Sci. (USA) 100:6934; Int'l Pat. App. Pub. WO 02/072636, issued as U.S. Pat. No. 7,833,525). The resulting high concentration, low viscosity crystals were obtained in high yield, and showed excellent physical and chemical stability, as well as retention of biological activity in vitro. However, the current application provides a method for producing a high concentration crystalline suspension. Subcutaneous injection of trastuzumab and infliximab crystalline suspensions resulted in an extended serum pharmacokinetic profile and high bioavailability compared with the soluble forms of the antibodies delivered intravenously. The crystalline formulation of trastuzumab was also effective in a preclinical model of human breast cancer. Spherical protein particles of therapeutic antibodies are disclosed at U.S. Pat. No. 7,998,477.

Crystallization by methods of the invention also provides an improved method of purification of anti-PD-1 antibodies. Although macro-scale crystallization is frequently used in purification of small organic molecules, there are few examples of the use of crystallization in the preparation of proteins. An exception is the use of a crystallization step in the manufacture of interferon alpha-2b (IFN-_{α2b}), where a temperature induction method is used in the purification process on a multigram scale. The resulting crystalline suspension is harvested by centrifugation, washed, and solubilized in a cold normal saline phosphate buffer. The crystallization and harvesting process removes small molecule, interferon-related and non-interferon impurities that may remain in the mother liquor or wash. Crystallization also confirms the purity of the therapeutic protein.

Some of these techniques were used to prepare pembrolizumab crystals of the invention, as described in greater detail in the Examples.

In particular embodiments of any of the methods described herein, the crystallization solution is produced by vapor diffusion or batch crystallization. In some embodiments of any of the methods described herein, the crystallization solution is produced by vapor diffusion, batch crystallization, or dialysis.

In another embodiment, the crystallization solution is produced by batch crystallization, wherein the batch crystallization is semi-continuous. In another embodiment, the crystallization solution is produced by semi-continuous batch operations resulting in improved filterability. The improved partciles's filterability is shown through a steady filtration flux.

In one embodiment, the aqueous buffered solution is Protein A product (PAP), Filtered Neutralized Viral Inactivated Pool (FNVIP) and AEX Product (AEXP) or Ultrafiltration Product (UFP).

In particular embodiments of any of the methods of producing crystalline anti-PD-1 monoclonal antibody described herein, the method further comprises the step of seeding the crystallization solution with crystals of the anti-PD-1 mAb prior to or during the incubation step.

The anti-PD-1 mAb crystals may be analyzed by various methods to examine or characterize their physical properties, such as crystal size, shape, surface morphology, total surface area and porosity. Such analytical techniques include, e.g., electron diffraction and solid-state nuclear magnetic resonance (ssNMR), light microscopy, transmission electron microscopy, scanning electron microscopy, atomic force microscopy, and various light scattering techniques. In addition, the biological activity and/or biophysical properties of the anti-PD-1 mAb in crystals of the invention may be analyzed by “re-dissolving” or solubilizing the antibody crystal in a buffer suitable for the desired analytical technique. For example, the solubilized anti-PD-1 mAb may be analyzed by one or more of ELISA, size exclusion chromatography, SDS PAGE, and dynamic light scattering.

In one aspect, the invention includes methods of treating a patient with cancer by administering 360 mg of the anti-PD-1 antibody or antigen binding fragment thereof. In specific embodiments, the patient is administered 370 mg of the anti-PD-1 antibody or antigen binding fragment thereof. In specific embodiments, the patient is administered 375 mg of the anti-PD-1 antibody or antigen binding fragment thereof. In specific embodiments the patient is administered 380 mg of the anti-PD-1 antibody or antigen binding fragment thereof. In specific embodiments, the patient is administered 385 mg of the anti-PD-1 antibody or antigen binding fragment thereof. In specific embodiments, the patient is administered 390 mg of the anti-PD-1 antibody or antigen binding fragment thereof. In specific embodiments, the patient is administered 395 mg of the anti-PD-1 antibody or antigen binding fragment thereof. In specific embodiments, the patient is administered 400 mg of the anti-PD-1 antibody or antigen binding fragment thereof. In specific embodiments, the patient is administered 420 mg of the anti-PD-1 antibody or antigen binding fragment thereof.

### IV. Methods for Producing a High Concentration Crystalline Suspension of an Anti-PD-1 Crystalline Antibody

In various embodiments, the crystalline suspension enables improved purification, storage, and therapeutic administration of the anti-PD-1 antibody.

In one aspect, the invention relates to methods of manufacturing a formulation comprising a high concentration crystalline suspension of pembrolizumab, wherein the crystalline suspension has a higher concentration and lower viscosity than would be possible with a corresponding non-crystalline solution at the same concentration of pembrolizumab.

In some embodiments the crystalline suspension of anti-PD-1 antibody crystals has an antibody concentration of greater than about 300 mg/ml. In other embodiments, the anti-PD-1 antibody crystals of the present invention have increased stability, i.e., they maintain biological activity of the anti-PD-1 antibody longer than corresponding solution formulations. In some embodiments, the increased stability is at room temperature, enabling storage of the crystalline suspensions of the present invention at room temperature rather than at 4° C. (weeks to months).

In a further aspect, the invention provides methods of purifying anti-PD-1 antibodies comprising crystallizing the antibody, using methods of the present invention, and then re-dissolving the antibody prior to use. In another aspect, the invention provides preparations of anti-PD-1 antibodies that have been purified by the crystallization methods described herein. In yet a further aspect, the invention provides methods of preparing crystalline anti-PD-1 antibodies for use in structure determination, e.g., by X-ray or Electron diffraction methods.

In some embodiments, excipient(s) are added directly to the crystallization liquid during or after crystallization. In other embodiments, the crystals are first harvested from the liquid, washed by suspension in a stabilizing solution, harvested from the stabilizing solution and then suspended in a liquid solution which comprises the excipient(s). The composition of the liquid may be any pharmaceutically acceptable medium, and may include, e.g., aqueous solutions and water in oil mixtures.

Pharmaceutical compositions of crystals made by the method described herein may be prepared by drying a liquid suspension comprising the crystals and the desired excipient(s), e.g., by passing a stream of nitrogen, air, or inert gas over the crystals, by air drying, vacuum drying or lyophilization. The moisture content in the final product will typically be less than 10%, 7%, 5% or 3% by weight.

A pharmaceutical composition comprising pembrolizumab that has been solubilized from pembrolizumab crystals in the method described herein may be prepared by adding a desired quantity of the crystals to a pharmaceutically acceptable dissolution buffer and incubating at 4°-22 C until the crystals have dissolved. In an embodiment, the dissolution buffer comprises 7 mM histidine, pH 5.6, 0.02% polysorbate 80 w/v and up to 7% sucrose w/v. In an embodiment, any particulates in the resulting composition are removed prior to administration, e.g., by centrifugation or filtration. In yet another embodiment, the dissolution buffer comprises comprises 7 mM histidine, pH 5.6, 0.02% polysorbate 80 w/v, 10 mM L-methionine and up to 7% sucrose w/v. In yet another embodiment, the dissolution buffer comprises 7 mM histidine, 0.02% polysorbate 80 w/v, 10 mM L-methionine and 10 mM HEPES, pH 7.0.

In yet another embodiment, the dissolution buffer comprises 10 mM L-methionine, 10 mM histidine, pH 5.5, 7% sucrose, and 0.02% polysorbate 80.

In particular embodiments, the pharmaceutical composition is a crystalline suspension and the concentration of the anti-PD-1 mAb is from about 150-300 mg/mL. In an additional embodiment, the pharmaceutical composition is a crystalline suspension and the concentration of the anti-PD-1 mAb is from about 150 to 250 mg/mL. In additional embodiments, the concentration of the anti-PD-1 mAb is ≥150 mg/mL, ≥175 mg/mL, ≥200 mg/mL, ≥225 mg/mL, >250 mg/mL, ≥275 mg/mL, about ≥300 mg/mL, about ≥325 mg/mL, about ≥350 mg/mL, about ≥375 mg/mL, about ≥400 mg/mL, about ≥425 mg/mL, about ≥450 mg/mL, about ≥475 mg/mL, or about ≥500 mg/mL.

In particular embodiments, the pharmaceutical composition produced by the methods described herein further include about 5 mM to about 200 mM buffer. In some embodiments, the amount of buffer is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. In one embodiment, the amount of buffer is about 10 mM. In another embodiment, the amount of buffer is about 100 mM.

In specific embodiments, the pharmaceutical compositions of the methods described herein further comprise about 0.01% to about 0.10% w/v non-ionic surfactant. In some embodiments, the amount of non-ionic surfactant is from about 0.01% to about 0.05% w/v, about 0.01% to about 0.04% w/v, 0.02% to about 0.05% w/v, or 0.02% to about 0.04% w/v. In further embodiments, the pharmaceutical compositions of the invention do not comprise any surfactant.

### V. Methods of Use

In one aspect, the invention relates to a method of treating cancer in a patient in need thereof, the method comprising administering to the subject an effective amount of a high concentration composition comprising from about 150-300 mg/mL anti-PD-1 mAb crystal described herein and a pharmaceutically acceptable carrier, to the patient. In some embodiments of the invention, the pembrolizumab crystal is dissolved into solution prior to administration to the patient (e.g., formulated as an aqueous formulation). In specific embodiments of this method, the composition is administered to the subject via intravenous administration.

In some embodiments of the methods of treatment herein, the dosage of anti-PD-1 mAb is 200 mg, which is administered to the patient about every 3 weeks. In alternative embodiments, the dosage of crystalline mAb is 400 mg, which is administered to the patient about every 6 weeks.

In another embodiments, the dosage of crystalline mAb is administered to the subject by subcutaneous injection. In one embodiment, the dosage of crystalline mAb is about 280 mg to about 450 mg, which is administered to the patient subcutaneously about every 3 weeks. In one embodiment, the dosage of crystalline mAb is 380 mg, which is administered to the patient subcutaneously about every 3 weeks.

In some embodiments of the invention, a composition comprising a high concentration of the pembrolizumab crystal, or pembrolizumab variant crystal, is administered to the patient once every three weeks for 12 weeks or more. In other embodiments, the composition of the invention or is administered to the patient once every three weeks for 15 weeks or more, 18 weeks or more, 21 weeks or more, 24 weeks or more, 27 weeks or more, 30 weeks or more, 33 weeks or more, 36 weeks or more, 39 weeks or more, 42 weeks or more, 45 weeks or more, 48 weeks or more, 51 weeks or more, 54 weeks or more, 57 weeks or more, 60 weeks or more, 63 weeks or more, 66 weeks or more, 69 weeks or more, 72 weeks or more, 75 weeks or more, 78 weeks or more, 81 weeks or more, 84 weeks or more, 87 weeks or more, or 90 weeks or more.

In other embodiments, a composition comprising a high concentration of the pembrolizumab crystal, or pembrolizumab variant crystal, is administered to the patient once every six weeks for 12 weeks or more. In other embodiments, the composition of the invention or is administered to the patient once every six weeks for 18 weeks or more, 24 weeks or more, 30 weeks or more, 36 weeks or more, 42 weeks or more, 48 weeks or more, 54 weeks or more, 60 weeks or more, 66 weeks or more, 72 weeks or more, 78 weeks or more, 84 weeks or more, 90 weeks or more, 96 weeks or more, 102 weeks or more, 108 weeks or more, 114 weeks or more, 120 weeks or more, 126 weeks or more, or 132 weeks or more.

In other embodiments of the invention, composition comprising a high concentration of the pembrolizumab crystal, or pembrolizumab variant crystal described herein, is administered to the patient intravenously or subcutaneously. In yet another embodiment, the composition comprising the pembrolizumab crystal, or pembrolizumab variant crystal, is delivered by inhalation or insufflation.

In some embodiments, the invention comprises a method of treating cancer in a human patient comprising administering a composition comprising a high concentration of a pembrolizumab crystal to the patient.

In one embodiment, the invention comprises a method of treating melanoma in a human patient comprising administering a composition comprising a high concentration of a pembrolizumab crystal to the patient. In a further embodiment, the melanoma is unresectable or metastatic. In yet a further embodiment, the melanoma is adjuvant melanoma. In specific embodiments, the melanoma is resected stage III melanoma.

In one embodiment, the invention comprises a method of treating metastatic non-small cell lung cancer (NSCLC) in a human patient comprising administering a composition comprising a high concentration of a pembrolizumab crystal to the patient. In a further embodiment, the NSCLC is squamous. In alternative embodiments, the NSCLC is non-squamous. In yet a further embodiment, the method further comprises administering carboplatin-paclitaxel or nab-paclitaxel to the patient. In another embodiment, the patient has a tumor with high PD-L1 expression [(Tumor Proportion Score (TPS) ≥50%)] and was not previously treated with platinum-containing chemotherapy. In yet another embodiment, the patient has a tumor with PD-L1 expression (TPS ≥1%) and was previously treated with platinum-containing chemotherapy. In another embodiment, the patient had disease progression on or after receiving platinum-containing chemotherapy. In another embodiment, the patient has a tumor with PD-L1 expression (TPS ≥1%) and was not previously treated with platinum-containing chemotherapy. In yet a further embodiment, the PD-L1 TPS is determined by an FDA-approved test. In another embodiment, the patient's tumor has no EGFR or ALK genomic aberrations. In a different embodiment, the patient's tumor has an EGFR or ALK genomic aberration and had disease progression on or after receiving treatment for the EGFR or ALK aberration(s) prior to receiving the anti-PD-1 antibody, or antigen binding fragment thereof.

In one embodiment, the invention comprises a method of treating metastatic non-small cell lung cancer (NSCLC) in a human patient comprising: (1) administering a composition comprising a high concentration of a pembrolizumab crystal to the patient, and (2) administering pemetrexed and carboplatin to the patient. In another embodiment, the patient was not previously treated with an anti-cancer therapeutic prior to starting the combination treatment regimen with the pembrolizumab crystal of the invention, in combination with pemetrexed and carboplatin. In certain embodiments, the patient has nonsquamous non-small cell lung cancer. In yet another embodiment, pemetrexed is administered to the patient in an amount of 500 mg/m². In another embodiment, pemetrexed is administered to the patient via intravenous infusion every 21 days. In specific embodiments, the infusion time is about 10 minutes. In another embodiment, the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally. In another embodiment, the invention further comprises administering about 1 mg of vitamin B₁₂ to the patient about 1 week prior to the first administration of pemetrexed and about every three cycles of pemetrexed administration (i.e., approximately every 9 weeks). In certain embodiments the vitamin B12 is administered intramuscularly. In another embodiment, the invention further comprises administering about 4 mg of dexamethasone to the patient twice a day on the day before, the day of, and the day after pemetrexed administration. In certain embodiments the dexamethasone is administered orally.

In one embodiment, the invention comprises a method of treating recurrent or metastatic head and neck squamous cell cancer (HNSCC) in a human patient comprising administering a composition comprising a high concentration of a pembrolizumab crystal to the patient. In certain sub-embodiments, the patient has recurrent or metastatic HNSCC.

In one embodiment, the patient was not previously treated with platinum-containing chemotherapy and the patient's tumor expresses PD-L1 (Combined Positive Score (CPS) ≥20).

In sub-embodiments, the patient was previously treated with platinum-containing chemotherapy. In certain embodiments, the patient had disease progression on or after platinum-containing chemotherapy.

In one embodiment, the invention comprises a method of treating refractory classical Hodgkin lymphoma (cHL) in a human patient comprising administering a composition comprising a high concentration of a pembrolizumab crystal to the patient. In one embodiment, the invention comprises a method of treating classical Hodgkin lymphoma (cHL) in a human patient comprising administering an effective amount of a pembrolizumab crystal of the invention to the patient, wherein the patient has relapsed after 3 or more lines of therapy for cHL.

In a sub-embodiment, the patient is an adult patient.

In alternative sub-embodiments, the patient is a pediatric patient.

In one embodiment, the invention comprises a method of treating locally advanced or metastatic urothelial carcinoma in a human patient comprising administering an effective amount of a pembrolizumab crystal of the invention to the patient.

In sub-embodiments, the patient is not eligible for cisplatin-containing chemotherapy.

In sub-embodiments, the patient has a tumor that expresses PD-L1. In some embodiments, the PD-L1 expression level is characterized by a CPS≥10.

In sub-embodiments, the patient has disease progression during or following platinum-containing chemotherapy or within 12 months of neoadjuvant or adjuvant treatment with platinum-containing chemotherapy.

In one embodiment, the invention comprises a method of treating unresectable or metastatic, microsatellite instability-high (MSI-H) or mismatch repair deficient solid tumors in a human patient comprising administering a composition comprising a high concentration of a pembrolizumab crystal to the patient.

In a sub-embodiment, the patient had disease progression following prior anti-cancer treatment.

In one embodiment, the invention comprises a method of treating unresectable or metastatic, microsatellite instability-high (MSI-H) or mismatch repair deficient colorectal cancer in a human patient comprising administering a composition comprising a high concentration of a pembrolizumab crystal to the patient.

In sub-embodiments, the patient had disease progression following prior treatment with a fluoropyrimidine, oxaliplatin, and irinotecan.

In one embodiment, the invention comprises a method of treating recurrent locally advanced or metastatic gastric cancer in a human patient comprising administering a composition comprising a high concentration of a pembrolizumab crystal to the patient.

In one embodiment, the invention comprises a method of treating recurrent locally advanced or metastatic gastroesophageal junction adenocarcinoma in a human patient comprising administering a composition comprising a high concentration of a pembrolizumab crystal to the patient.

In sub-embodiments, the patient's tumor expresses PD-L1 [Combined Positive Score (CPS) ≥1].

In sub-embodiments, the patient has disease progression on or after two or more prior lines of therapy including fluoropyrimidine- and platinum-containing chemotherapy.

In sub-embodiments, the patient has disease progression on or after two or more prior lines of therapy including HER2/neu-targeted therapy.

In one embodiment, the invention comprises a method of treating cervical cancer in a human patient comprising administering a composition comprising a high concentration of a pembrolizumab crystal to the patient. In a sub-embodiment, the patient has recurrent or metastatic cervical cancer.

In sub-embodiments, the patient had disease progression on or after chemotherapy.

In other sub-embodiments, the patient has a tumor that expresses PD-L1 [CPS≥1].

In one embodiment, the invention comprises a method of treating cancer in a human patient comprising administering a composition comprising a high concentration of a pembrolizumab crystal to the patient, wherein the patient has a cancer selected from the group consisting of: melanoma, non-small cell lung cancer, small cell lung cancer, head and neck cancer, urothelial cancer, breast cancer, gastrointestinal cancer, gastroesophageal junction adenocarcinoma, multiple myeloma, hepatocellular cancer, non-Hodgkin lymphoma, primary mediastinal large B-cell lymphoma, renal cancer, Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, and salivary cancer.

In one embodiment, the invention comprises a method of treating cancer in a human patient comprising administering a composition comprising a high concentration of a pembrolizumab crystal to the patient, wherein the patient has a cancer selected from the group consisting of: melanoma, non-small cell lung cancer, small cell lung cancer, head and neck cancer, urothelial cancer, breast cancer, gastric cancer, gastroesophageal junction adenocarcinoma, multiple myeloma, hepatocellular cancer, non-Hodgkin lymphoma, primary mediastinal large B-cell lymphoma (PMBCL), renal cancer, classical Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, salivary cancer.

In one embodiment, the invention comprises a method of treating cancer in a human patient comprising administering a composition comprising a high concentration of a pembrolizumab crystal to the patient, wherein the patient has a tumor having a high mutational burden.

In one embodiment, the invention comprises a method of treating cancer in a human patient comprising administering a composition comprising a high concentration of a pembrolizumab crystal to the patient, wherein the patient has a small-cell lung cancer. In a sub-embodiment, the patient has metastatic SCLC. In certain sub-embodiments, the patient was previously treated with platinum-based chemotherapy with disease progression on or after platinum-based chemotherapy and at least one other prior line of therapy. In certain sub-embodiments, the patient had disease progression on or after the platinum-based chemotherapy and at least one other prior line of therapy.

In one embodiment, the invention comprises a method of treating non-Hodgkin lymphoma in a human patient comprising administering a composition comprising a high concentration of a pembrolizumab crystal to the patient. In a sub-embodiment, the non-Hodgkin lymphoma is mediastinal large B-cell lymphoma. In some embodiments, the non-Hodgkin lymphoma is primary mediastinal large B-cell lymphoma (PMBCL) that is refractory. In other embodiments, the patients have PMBCL and has relapsed after 2 or more prior lines of therapy.

In one embodiment, the invention comprises a method of treating breast cancer in a human patient comprising administering a composition comprising a high concentration of a pembrolizumab crystal to the patient. In a sub-embodiment, the breast cancer is triple negative breast cancer. In a sub-embodiment, the breast cancer is ER+/HER2− breast cancer.

In one embodiment, the invention comprises a method of treating nasopharyngeal cancer in a human patient comprising administering a composition comprising a high concentration of a pembrolizumab crystal to the patient.

In one embodiment, the invention comprises a method of treating thyroid cancer in a human patient comprising administering a composition comprising a high concentration of a pembrolizumab crystal to the patient.

In one embodiment, the invention comprises a method of treating salivary cancer in a human patient comprising administering a composition comprising a high concentration of a pembrolizumab crystal to the patient.

In one embodiment, the invention comprises a method of treating Merkel cell carcinoma (MCC) in a human patient comprising administering a composition comprising a high concentration of a pembrolizumab crystal to the patient. In sub-embodiments the MCC is recurrent locally advanced or metastatic.

In one embodiment, the invention comprises a method of treating cancer in a human patient comprising administering a composition comprising a high concentration of a pembrolizumab crystal to the patient, wherein the cancer is selected from the group consisting of: melanoma, non-small cell lung cancer, relapsed or refractory classical Hodgkin lymphoma, head and neck squamous cell carcinoma, cervical cancer, urothelial cancer, esophageal cancer, gastric cancer, primary mediastinal large B-cell lymphoma, and hepatocellular carcinoma.

In one embodiment, the invention comprises a method of treating cancer in a human patient comprising administering a composition comprising a high concentration of a pembrolizumab crystal to the patient, wherein the cancer is a heme malignancy.

In one embodiment, the heme malignancy is selected from the group consisting of: acute lymphoblastic leukemia (ALL), acute myeloid leukemia (AML), chronic lymphocytic leukemia (CLL), chronic myeloid leukemia (CML), ddiffuse large B-cell lymphoma (DLBCL), EBV-positive DLBCL, primary mediastinal large B-cell lymphoma, T-cell/histiocyte-rich large B-cell lymphoma, follicular lymphoma, Hodgkin lymphoma (HL), mantle cell lymphoma (MCL), multiple myeloma (MM), myeloid cell leukemia-1 protein (MCL-1), myelodysplastic syndrome (MDS), non-Hodgkin lymphoma (NHL), and small lymphocytic lymphoma (SLL).

In one embodiment, the invention comprises a method of treating cancer in a human patient comprising administering a composition comprising a high concentration of a pembrolizumab crystal to the patient, wherein the patient has a tumor with a high mutational burden.

In one embodiment, the invention comprises a method of treating hepatocellular carcinoma in a human patient comprising administering a composition comprising a high concentration of a pembrolizumab crystal to the patient. In a sub-embodiment, the patient was previously treated with sorafenib.

In one embodiment, the invention comprises a method of treating renal cancer in a human patient comprising administering a composition comprising a high concentration of a pembrolizumab crystal to the patient. In sub-embodiments, the renal cancer is clear cell renal cell carcinoma.

In one embodiment, the invention comprises a method of treating esophageal cancer in a human patient comprising administering a composition comprising a high concentration of a pembrolizumab crystal to the patient. In a sub-embodiment, the esophageal cancer is recurrent locally advanced or metastatic squamous cell carcinoma of the esophagus. In a further sub-embodiment, the patient had disease progression after one or more lines of systemic therapy. In a further sub-embodiment, the patient's tumors express PD-L1 [Combined Positive Score (CPS) ≥10].

In one embodiment, the invention comprises a method of treating ovarian carcinoma in a human patient comprising administering a composition comprising a high concentration of a pembrolizumab crystal to the patient.

In one embodiment, the invention comprises a method of treating biliary tract cancer in a human patient comprising administering a composition comprising a high concentration of a pembrolizumab crystal to the patient. In a sub-embodiment, the cancer is locally advanced unresectable or metastatic biliary tract cancer. In some embodiments the patient is further treated with gemcitabine and cisplatin.

In one embodiment, the invention comprises a method of first-line treatment of adults with locally advanced unresectable or metastatic HER2-negative gastric or gastroesophageal junction (GEJ) adenocarcinoma in combination with fluoropyrimidine-and platinum-containing chemotherapy.

In one embodiment, the invention comprises a method of treating colorectal cancer in a human patient comprising administering a composition comprising a high concentration of a pembrolizumab crystal to the patient.

In any of the methods of the invention described herein, the “pembrolizumab crystal” or the “anti-PD-1 crystalline mAb” can be any pembrolizumab crystals, or pembrolizumab variant crystals described herein (i.e., a crystal described herein or made by the methods described herein), as described in Section II of the Detailed Description of the Invention herein, entitled “Anti-PD-1 Antibodies for Use in the Methods of the Invention” or as described in Section IV, entitled “Anti-PD-1 Crystalline Antibody Suspensions and Compositions.”

Malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

In some embodiments, the compositions of the invention are administered to a subject having a cancer characterized by elevated expression of PD-L1 and/or PD-L2 in tested tissue samples, including ovarian, renal, colorectal, pancreatic, breast, liver, gastric, esophageal cancers, and melanoma. Additional cancers that can benefit from treatment with the compositions of the invention include those associated with persistent infection with viruses such as human immunodeficiency viruses, hepatitis viruses class A, B and C, Epstein Barr virus, human papilloma viruses that are known to be causally related to for instance Kaposi's sarcoma, liver cancer, nasopharyngeal cancer, lymphoma, cervical, vulval, anal, penile, and oral cancers.

Additional aspects include methods of using a pharmaceutical composition of the invention to treat a patient having, suspected of having, or at risk for having an infection or infectious disease. Thus, the invention provides a method for treating chronic infection in a mammalian subject comprising administering a composition comprising a high concentration of anti-PD-1 crystalline mAb described herein to the subject. In some specific embodiments of this method, the composition is administered to the subject via intravenous administration. In other embodiments, the composition is administered to the subject by subcutaneous administration.

In this aspect, the compositions of the invention can be used alone, or in combination with vaccines, to stimulate the immune response to pathogens, toxins, and self-antigens. The compositions of the invention can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Compositions of the invention that comprise antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The anti-PD-1 mAb crystalline compositions of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), a growth inhibitory agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist. In still further embodiments, the additional therapeutic agent is a PARP inhibitor. In still further embodiments, the additional therapeutic agent is a multi-tyrosine kinase inhibitor. In additional embodiments, the additional therapeutic agent is a MEK inhibitor. In additional embodiments, the additional therapeutic agent is a CXCR2 antagonist. In additional embodiments, the additional therapeutic agent is navarixin. In additional embodiments, the additional therapeutic agent is olarparib. In additional embodiments, the additional therapeutic agent is selumetinib. In additional embodiments, the additional therapeutic agent is axitinib.

Suitable routes of administration for the additional therapeutic agent may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intra-arterial or intravenous injection.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g., biotherapeutic or chemotherapeutic agent) will depend in part on the particular therapeutic agent, the severity of the cancer being treated, and patient characteristics. Guidance in selecting appropriate doses of antibodies, cytokines, and small molecules is available. See, e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY.

In some embodiments, the additional therapeutic agent is administered by continuous infusion, or by doses at intervals of, e.g., one day, 1-7 times per week, one week, two weeks, three weeks, monthly, bimonthly, etc. In some embodiments, the dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

In certain embodiments, dosing will comprise administering to a subject escalating doses of 1.0, 3.0, and 10 mg/kg of the additional therapeutic agent, over the course of treatment. The formulation can be a reconstituted liquid formulation, or it can be a liquid formulation not previously lyophilized. Time courses can vary and can continue as long as desired effects are obtained. In certain embodiments, dose escalation will continue up to a dose of about 10 mg/kg. In certain embodiments, the subject will have a histological or cytological diagnosis of melanoma, or other form of solid tumor, and in certain instances, a subject may have non-measurable disease. In certain embodiments, the subject will have been treated with other chemotherapeutics, while in other embodiments, the subject will be treatment naïve.

In certain embodiments, the dosing regimen will comprise administering a dose of from about 0.005 mg/kg to about 10 mg/kg, with intra-patient dose escalation. In certain embodiments, a dose of 5 mg/kg or 10 mg/kg will be administered at intervals of every 3 weeks, or every 2 weeks. In yet additional embodiments, a dose of 3 mg/kg will be administered at three-week intervals for melanoma patients or patients with other solid tumors. In these embodiments, patients should have non-resectable disease; however, patients may have had previous surgery.

Subcutaneous administration may be performed by injection using a syringe, or using other injection devices (e.g., the Inject-ease® device); injector pens; or needleless devices (e.g., MediJector and BioJector®).

Embodiments of the invention also include formulations comprising a high concentration of the crystals described herein (crystalline pembrolizumab or a pembrolizumab variant) or made by the methods described herein (i) for use in, (ii) for use as a medicament or composition for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of PD-1-related disease; (g) halting or delaying the progression cancer; (h) stabilization of PD-1-related disease; (i) inhibiting the growth or survival of tumor cells; (j) eliminating or reducing the size of one or more cancerous lesions or tumors; (k) reduction of the progression, onset or severity of PD-1-related disease; (1) reducing the severity or duration of the clinical symptoms of PD-1-related disease such as cancer (m) prolonging the survival of a patient relative to the expected survival in a similar untreated patient n) inducing complete or partial remission of a cancerous condition or other PD-1 related disease, (o) treatment of cancer, or (p) treatment of infection or infectious disease.

Pembrolizumab crystalline suspensions are filtered and washed with process relevant liquids without alteration of crystallinity as confirmed by unchanged x-ray diffraction patterns from SAXS analysis. The wash liquids are process relevant aqueous pH buffer solutions prepared from, but not limited to, HEPES or TRIS, and containing no additional component. The wash liquids are process relevant aqueous pH buffer solutions prepared from, but not limited to, HEPES or TRIS, and further comprising one or more of the initial crystallization components (precipitants, additives, excipients, etc.) including, but not limited to, PEG, caffeine, and DSS.

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### VI. EXAMPLES

### Example 1

### Preparation of Lyo Cake for Crystalline Pembrolizumab Experiments:

A mixture of 50 mg/ml pembrolizumab and 10 mM histidine, pH 5.4, (8 ml) was dialyzed in a 100 KDa cutoff dialysis bag versus 100 ml of distilled water and exchanged 2× over 18 hours. The resulting volume increased to about 9 ml total during dialysis. 0.256 ml of 50% PEG 3350 (W/V) solution was added to a final 1.55% PEG 3350 concentration. The resulting solution at 30 mg/ml pembrolizumab by UV A260/280 nm was added to 32×2 ml sterile vials, each fitted with a septum cap. A lyophilization recipe run in a Lyo Star 2 FTS Systems from SP Scientific Products, Warminster, PA is shown below in Table 3.

TABLE 3
Parameters for preparation of lyophilize cake for
crystalline pembrolizumab monoclonal antibody.

| | | Batch 1 | |
|---|---|---|---|
| | | Size | |
| Parameters | | 32 vials | |
| Freezing | Temp | −50° C., Ramp at 1° C. per minute | |
| | Time | 8 | hours |
| Primary Drying | Temp | −29° C., Ramp at 0.5° C. per minute | |
| | Time | 36 | hours |
| | Vacuum | 90 | μm Hg |
| Secondary Drying | Temp | 30° C., Ramp at 1° C. per minutes | |
| | Time | 8 | hours |
| | Vacuum | 90 | μm Hg |

End Cycle, backfill with nitrogen, stopper, and seal

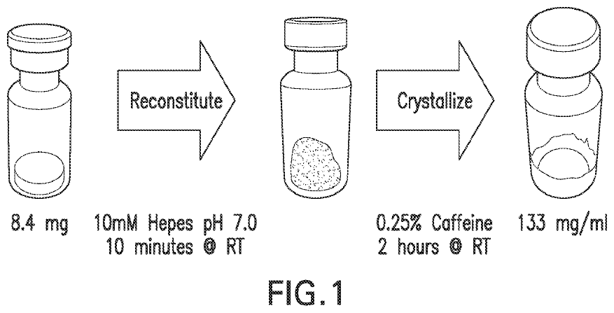

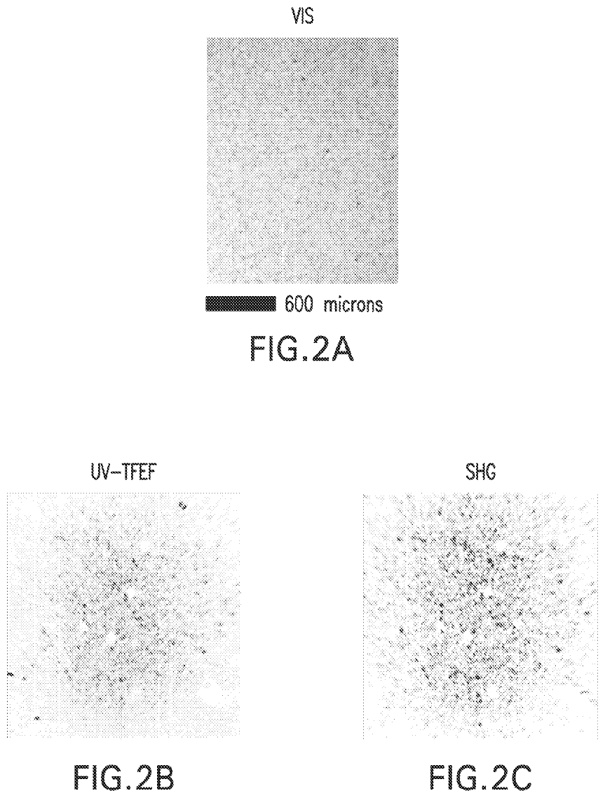

57 μl of 10 mM HEPES, pH 7.0 was added to the resulting cake (8.4 mg) via a syringe. After a reconstitution time of 10 minutes at room temperature, the cake dissolved completely and had a consistency similar to that of mineral oil as shown in the middle panel of FIG. 1. For crystallization, 6 μl of 25 mg/ml caffeine (0.25% caffeine) in 10 mM histidine solution was subsequently added and the vial was mixed on a MediMix100® automated mixer (Health mark, CA) set at the lowest speed at room temperature. The clear solution was observed to change to a white paste slowly within 2 hours. The final crystalline suspension was measured at 133 mg/ml by 1/10 dilution in PBS by UV A260/280 nm reading. SONICC analyses shown in FIG. 2 was run on a on I/O dilution (10 mM Hepes, pH 7.0, 8% PEG 3350) of the resulting paste confirming conversion to a crystalline suspension as shown in the right panel of FIG. 1. This was a 10-fold increase in concentration relative to the standard 13 mg/ml crystallization conditions reported in WO2020/092233.

### Example 2

### Example 2A: Lyo Cake Pembrolizumab Crystallization Screening: >200 mg/Ml Lyo Cake Reconstitution Plan

To 1 to 5 lyo cake vials (Example 1) was added 190 μl 10 mM Hepes, pH 7.0 followed by 20 μl of 25 mg/ml caffeine, 10 mM histidine, pH 5.4 as described in Table 4 below. The resulting solution was mixed by aspiration multiple times and placed on Redi-mixer at room temperature for 2 hours.

Crystals were visualized using a SONICC imaging system from Formulatrix (Bedford, MA, USA). The SONICC system has two imaging methods, Second Harmonic Generation (SHG), which probes crystallinity, and Ultraviolet Two-Photon Excited Fluorescence (UV-TPEF), which is specific to proteinaceous samples. A SONICC image was run on an aliquot which confirmed crystallinity based on a comparison of the VIS, UV and SHG imaging shown in Example 2B.

TABLE 4
pembrolizumab crystallization screening

| | | | 25 mg/ml | | Final |
|---|---|---|---|---|---|
| # Vials | | 10 mM Hepes, | caffeine, 10 mM | Final | Concen- |
| (total | | pH 7.0 | histidine, pH 5.4 | Volume | tration |
| mgs) | | (μl) | (ul) | (μl) | (mg/ml) |
| 1 | (8.4 mg) | 57 | 6 | 63 | 133 |
| 5 | (42 mg) | 190 | 20 | 210 | 200 |
| 5 | (42 mg) | 142 | 15 | 157 | 266 |

### Example 2B: SONICC Characterization of 200 mg/ml Crystalline Pembrolizumab Suspension Derived from Example 2A

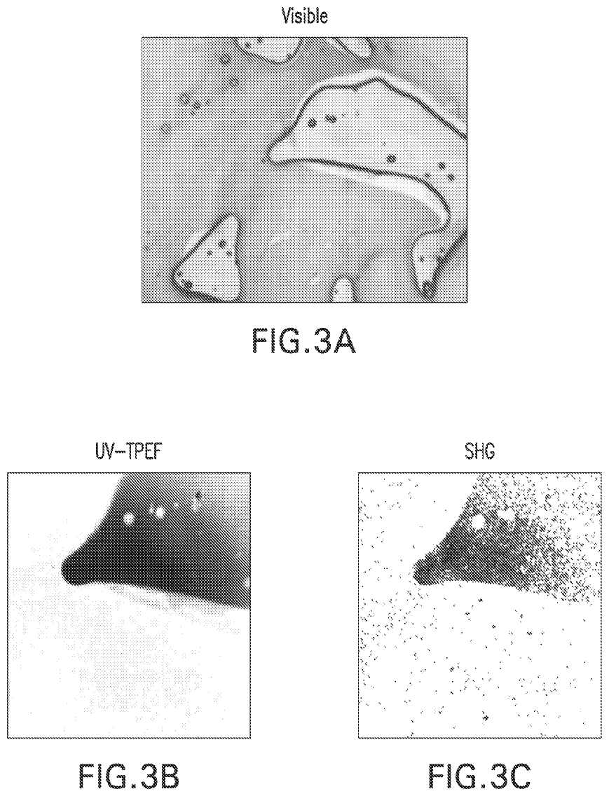

2 μl of the suspension from Example 2A was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW) settings). The visible image is shown in the left panel of FIG. 3, the UV-TPEF image is shown in the middle image of FIG. 3, and the SHG image is shown in the right panel of FIG. 3. The sample was UV+ and SHG+ consistent with a chiral crystalline suspension.

### Example 3

### Example 3A: Pembrolizumab Dialysis Crystallization Experiments

200 μl of pembrolizumab at 200 mg/ml in 10 mM histidine, 10 mM methionine, pH 5.4 was dialyzed in a 0.5 ml 50K MW cutoff dialysis tube against a stirred 100 ml of 10 mM Hepes, pH 6.8, 6% PEG 3350 buffers at room temperature. After several minutes, noticeable precipitation was observed on the top of the liquid in the dialysis bag (amorphous solid). The precipitation increased within several hours to a heavy visible precipitate throughout the bag. A sample (10 μl) was taken for SONICC analyses.

The dialysate was charged to a stirred 100 ml of 10 mM Hepes, pH 6.8, 6% PEG 3350, 2.5 mg/ml caffeine buffer at room temperature. After 18 hours, a 10 μl aliquot was taken for SONICC analyses. 2 μl of the suspensions was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW)) settings shown in Example 3B. Crystalline suspension was confirmed by SONICC. The upper panel was UV+, SHG-consistent with an amorphous solid whereas and lower panel was UV+ and SHG+ consistent with a chiral crystalline suspension.

### Example 3B: SONICC Characterization of Example 3A

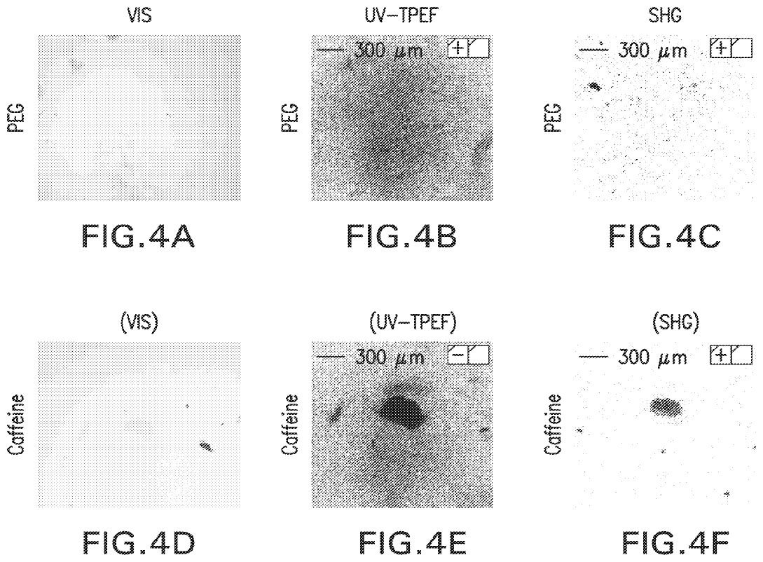

2 μl of the suspension from Example 3A was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW) settings). SONICC results for Example 3B are shown in FIG. 4. The upper row in FIG. 4 shows a sample from the PEG only solution indicating an amorphous solid; UV+ and SHG-shown in FIG. 4A, FIG. 4B, and FIG. 4C. The lower row (FIG. 4D, FIG. 4E, and FIG. 4F) shows a sample taken from the caffeine buffer suspension consistent with a crystalline suspension; UV+ and SHG+.

### Example 4

### High Concentration Anti-PD-1 Batch Crystallization Screening Experiments:

Pembrolizumab at 50 mg/ml in 10 mM histidine, pH 5.4, 10 mM methionine aliquots (control pembrolizumab) were used to investigate higher mAb final crystallization conditions to produce a 200 mg/ml formulation in situ method. The experimental conditions at the 100 μl scale were run in 1 ml Eppendorf tubes at room temperature and mixed using a Labnet rotisserie for 2-18 hours. The resulting crystalline suspensions were allowed to settle, and the supernatants were decanted 4×. The resulting mixtures were characterized by UV 260/280 nm protein determination and SONICC analyses (visible; (5 mega pixels), UV-TFEF (standard) and SHG (450 mW) settings). The results are shown in Table 5.

TABLE 5
Screening conditions and resulting mixtures characterizations

| Pembrolizumab | PEG 3350 | 25 mg/ml | Total | Reaction | Decant | Crystallinity |
|---|---|---|---|---|---|---|
| (mg/ml) | (%) | Caffeine | Protein | Conditions | 4X | Confirmation |
| μl | μl | μl | (mg) | (mg/ml) | (mg/ml) | SONICC |
| Control | (10%) | 10 | 1.4 | 13 | 51 | SHG+ |
| Pembrolizumab | 66 | | | | | |
| (44) | | | | | | |
| 33 | | | | | | |
| (50) | (10%) | 10 | 1.65 | 15 | 60 | SHG+ |
| 33 | 66 | | | | | |
| (100) | (10%) | 20 | 3.3 | 26 | 100 | SHG+ |
| 33 | 72 | | | | | |
| (150) | (10%) | 30 | 5.0 | 35 | 140 | SHG+ |
| 33 | 78 | | | | | |
| (>150) | (10%) | 40 | >5 | >35 | >140 | SHG+ |
| 33 | 84 | | | | | |
| (150) | (20%) | 40 | 5 | 44 | 176 | Mix |
| 33 | 40 | | | | | Amorphous |
| | | | | | | crystals |
| (150) | (20%) | 40 | 6 | 50 | 200 mg/ml | Mix |
| 40 | 40 | | | | | Amorphous |
| | | | | | | crystals |
| (>150) | (20%) | 40 | >5 | >50 | >200 | Mix |
| 33 | 33 | | | | | Amorphous |
| | | | | | | crystals |
| (>150) | (20%) | 40 | >6 | >50 | >200 | Mix |
| 40 | 40 μl | | | | | Amorphous |
| | | | | | | crystals |
| (150) | (10%) | 40 | 9.9 | 52 | | Mix |
| 66 | 84 | | | | | Amorphous |
| | | | | | | crystals |
| (>150) | (10%) | 40 | >9.9 | >52 | | Mix |
| 66 | 84 | | | | | Amorphous |
| | | | | | | crystals |

### Example 5

Pembrolizumab Batch Crystallization Experiments at 200 mg/ml

30 ml of pembrolizumab at 50 mg/ml in 10 mM histidine, pH 5.4, 10 mM methionine was concentrated using 2 Amicon Ultra 15 100K cutoff centrifugal units. The units were centrifuged in a Beckman Coulter centrifuge Allegra X-15R at 3500 RPM for 2 hours at 4° C. The resulting concentrate protein concentration was 240 mg/ml, 10 mM histidine pH 5.4 by 260/280 nm UV protein concentration. PEG3350 50 mM hepes, pH 7.0 solutions were added to the pembrolizumab solutions followed by the 25 mg/ml caffeine, 10 mM histidine, pH 5.4 solution further illustrated in Table 6. Crystallization reactions were performed in 1.5 ml Eppendorf tubes and placed on a hematology/chemistry mixer (Medmark Technologies) at 24 RPM for 2 hours at room temperature. Resulting crystalline suspensions were stored at 4° C. SONICC characterization were run on the suspensions (visible; (5 mega pixels), UV-TFEF (standard) and SHG (450 mW) settings) and determined to be consistent with a crystalline suspension.

TABLE 6
Batch crystallization experiments and conditions for Example 5

| 240 mg/ml | | 25 mg/ml | |
|---|---|---|---|
| Pembrolizumab | | Caffeine | |
| 10 mM Histidine, | PEG 3350 | 10 mM | Charac- |
| pH 5.4 | 50 mM Hepes, | Histidine, | terization |
| (ul) | pH 7.0 | pH 5.4 | (SONICC) |
| 83 | 10 μL (47.5% PEG, 50 mM) | 7 μl | SHG+ |
| | Final 4.8%, 5 mM | (Final | |
| | | 0.17%) | |
| 83 | 10 μL (30% PEG, 150 mM) | 7 μl | SHG+ |
| | Final 3%, 15 mM | (Final | |
| | | 0.17%) | |
| 83 | 10 μL (20% PEG, 150 mM | 7 μl | SHG+ |
| | Hepes, 7.0) | (Final | |
| | Final 2%, 15 mM | 0.17%) | |
| 83 | 10 μL (10% PEG, 150 mM | 7 μl | SHG+ |
| | Hepes, 7.0) | (Final | |
| | Final 1%, 15 mM | 0.17%) | |
| 83 | 10 μL (150 mM Hepes, 7.0) | 7 μl | SHG+ |
| | Final 0%, 15 mM | (Final | |
| | | 0.17%) | |

### Example 6

### Example 6A: High Concentration Pembrolizumab Batch Crystallization at 200 mg/ml

30 ml of pembrolizumab at 50 mg/ml in 10 mM histidine, pH 5.4, 10 mM methionine was concentrated using 2 Amicon Ultra 15 100K cutoff centrifugal units. The units were centrifuged in a Beckman Coulter centrifuge Allegra X-15R at 3500 RPM for 2 hours at 4° C. The resulting concentrate protein concentration was 240 mg/ml, 10 mM histidine pH 5.4 by 260/280 nm UV protein concentration. Either i) 100 μL of 10% PEG3350 150 mM Hepes, pH 7.0 solutions was added to the 830 μL of pembrolizumab solution followed by 70 μL of 25 mg/ml caffeine, 10 mM histidine, pH 5.4 solution or ii) 100 μL 150 mM Hepes, pH 7.0 solutions were added to the 830 μl of pembrolizumab solution followed by the 70 μl of 25 mg/ml caffeine, 10 mM histidine, pH 5.4 solution. Crystallization reactions were performed in 5 ml sterile vials fitted with a septum cap. A hematology/chemistry mixer (Medmark Technologies) was used to mix the vials at 24 RPM for 2 hours at room temperature. SONICC characterization were run on the suspensions (visible; (5 mega pixels), UV-TFEF (standard) and SHG (450 mW)) settings and determined to be consistent with a crystalline suspension as shown in Example 6B.

### Example 6B: SONICC Characterization of Example 6A

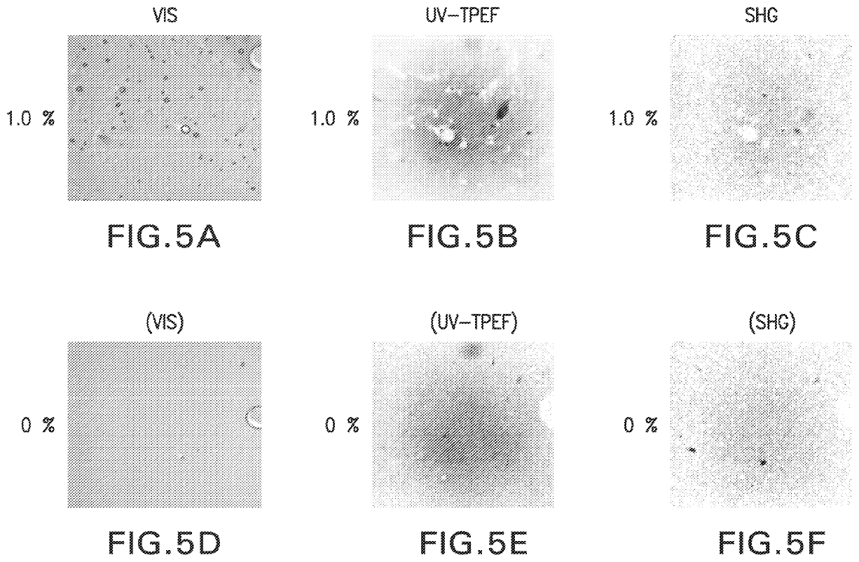

2 μl of the suspension from Example 3 was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW)) settings shown in Example 6A. The upper and lower panels were UV+ and SHG+ consistent with a chiral crystalline suspension. Results are shown in FIG. 5.

### Example 7

### Example 7A: High Concentration Pembrolizumab Batch Crystallization at 209 mg/Ml (1 Ml Scale)

30 ml of pembrolizumab at 50 mg/ml in 10 mM histidine, pH 5.4, 10 mM methionine was concentrated using 2 Amicon Ultra 15 100K cutoff centrifugal units. The units were centrifuged in a Beckman Coulter centrifuge Allegra X-15R at 3500 RPM for 2 hours at 4° C. The resulting concentrate protein concentration was 240 mg/ml, 10 mM histidine pH 5.4 by 260/280 nm UV protein concentration. 10 ml of concentrate was dialyzed in Spectra/Pore Ce Cellulose acetate 10 mm diameter 8000 MW cutoff dialyzer vs. 100 ml of 50 mM Hepes, pH 7 (2 exchanges) over 24 hours. The resulting solution 930 μl of 225 mg/ml pembrolizumab in 50 mm Hepes, pH 7.0 was placed into a 5 ml sterile vials fitted with a septum cap. A hematology/chemistry mixer (Medmark Technologies) was used to mix the vials at 24 RPM for 2 hours at room temperature. Results and recipe for the experiment shown in Table 7. SONICC characterization was run on an aliquot described in Example 7B. SONICC characterization were run on the suspensions (visible; (5 mega pixels), UV-TFEF (standard) and SHG (450 mW)) settings and determined to be consistent with a crystalline suspension as compared to a control crystalline suspension.

TABLE 7
Summary of the experimental conditions and results

| Pembrolizumab | | | |
|---|---|---|---|
| 50 mM Hepes, | 25 mg/ml | | |
| pH 7.0 | Caffeine, 10 mM | Final | Characterization |
| 225 mg/ml | Histidine, pH 5.4 | Concentration | (SONICC/yield) |
| 930 μl | 70 μl (Final 1.7 | 209 mg/ml | SHG + |
| (157 mg/ml) | mg/ml caffeine) | | 95% yield |
| | | | SS-NMR ✓ |
| | | | IEX and SEC ✓ |

### Example 7B: SONICC Characterization of Crystalline Suspension

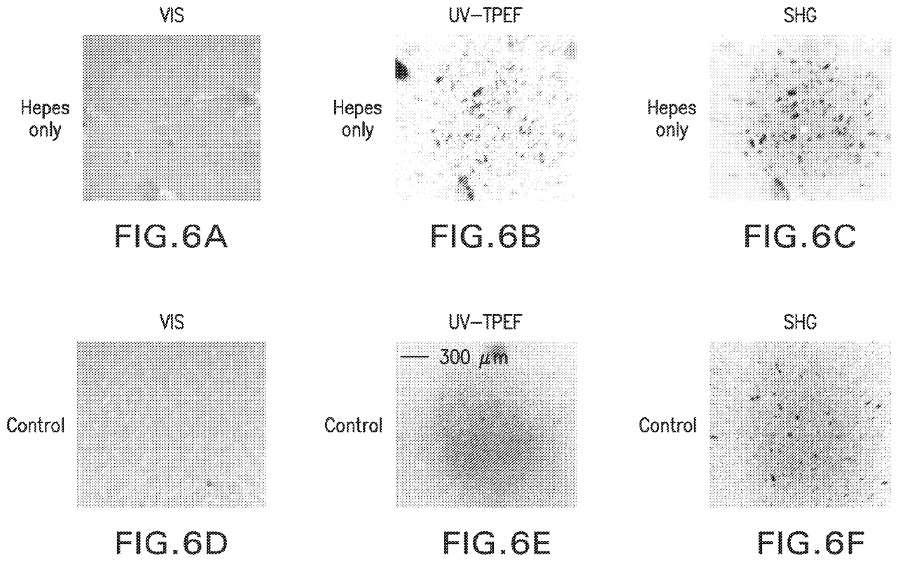

2 μl of the suspension from Example 7A was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW) settings). The upper and lower panels were UV+ and SHG+ consistent with a chiral crystalline suspension. Results are shown in FIG. 6.

### Example 7C: Solid-State NMR Spectroscopy of Crystalline Pembrolizumab Suspension Derived in Example 7A

Solid-state NMR spectra are acquired on a Bruker Avance III HD 400 MHZ spectrometer equipped with a 4.0 mm H/F/X magic angle spinning (MAS) probe. The probes are tuned to double resonance C/H for ¹³C (carbon-13) experiments on the 400 MHZ. The MAS frequency for all experiments is 12 kHz. The sample temperature is controlled at 10° C., ¹³C cross polarization (CP) MAS spectra are collected under 90.9 kHz 1H dipolar decoupling during acquisition, with a CP contact time of 1 millisecond and a recycle delay of 2 seconds. ¹³C chemical shifts are referenced to the ¹³C signal of the carbonyl carbon of glycine (alpha-form) at 176.49 ppm. For purposes of solid-state NMR, the term “about” mean±0.10 ppm.

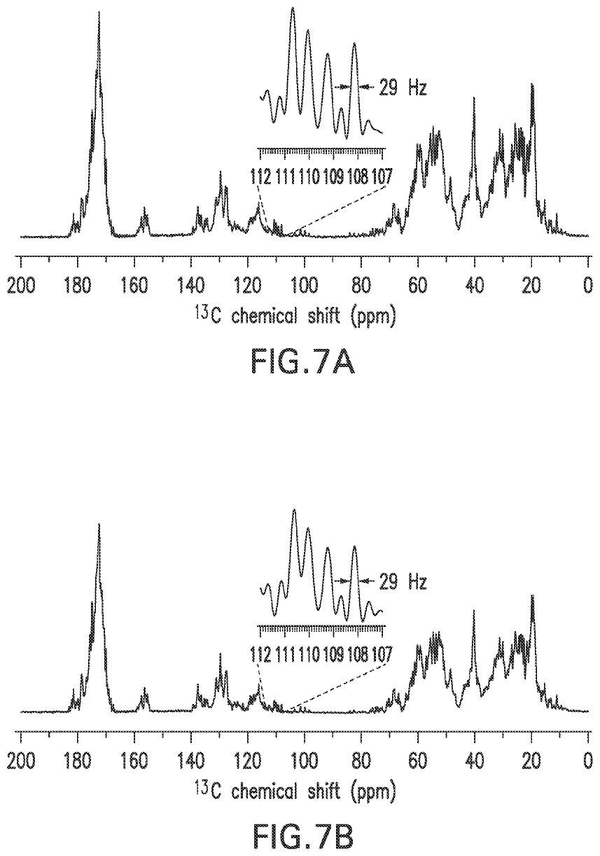

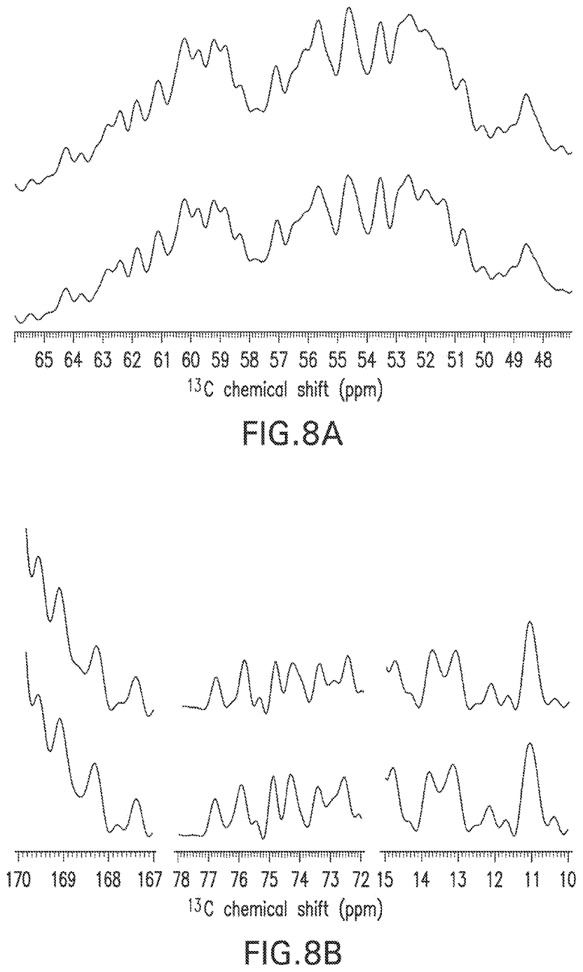

Using the solid state ¹³C 400 MHz NMR equipment and procedures described above, the crystalline pembrolizumab suspension has been measured. The ¹³C CP MAS NMR spectra for the crystalline pembrolizumab suspension grown with and without PEG was obtained. The full spectra and a few enlarged regions are respectively shown in FIG. 7A and FIG. 7B, and FIGS. 8A and 8B. The crystalline pembrolizumab suspension grown with PEG is shown n FIG. 7A and FIG. 8A and the crystalline pembrolizumab suspension grown without PEG is shown in FIG. 7B and FIG. 8B. Characteristic peaks for the pembrolizumab-caffeine crystal are observed at about 181.45, 179.91, 170.05, 169.11, 108.15, 109.24, 110.69, 101.42, 99.93, 82.54, 84.08, 13.78, 13.13 and 11.11 ppm.

### Example 7D: Size Exclusion Chromatography Multi-Angle Scattering Analyses of Redissolved Crystalline Pembrolizumab Suspension Derived from Example 7A

Size Exclusion Chromatography Multi-Angle Light Scattering (SEC-MALS) is utilized to determine the molecular weight (MW). This method utilizes the Waters Acquity UPLC H-Class Bio system (attached to a Wyatt UP LS laser and UP tRex detector) with a Waters Acquity BEH200 SEC column to separate molecules based on their hydrodynamic radius. Resolved peaks are detected by the absorbance at 280 nm, light scattering intensity, and dRI (differential Refractive Index). This method is run under isocratic conditions at a flow rate (FR) of 0.5 mL/min in a buffer composed of 50 mM phosphate, 450 mM arginine, pH 7.0. SEC-MALS results as shown in Table 8.

TABLE 8
Results of SEC analyses Example 7B

| Sample | % HMW | % Main peak |
|---|---|---|
| Pembrolizumab, 50 mM Hepes, pH 7.0 | 0.60 | 99.4 |
| 225 mg/ml (starting material) | | |
| Crystalline suspension, 209 mg/ml (final) | 0.55 | 99.5 |

### Example 7E: Ion Exchange Chromatography Analyses of Redissolved Crystalline Pembrolizumab Suspension Derived from Example 7B

Ion Exchange Chromatography provides an ion exchange HPLC method to determine charge variants present in pembrolizumab formulation. Analysis is performed using a Dionex ProPac® WCX-10, 10 μm 4×250 mm column and a mobile phase gradient from 24 mM MES pH 6.1, 4% acetonitrile to 20 mM NaPO₄, 95 mM NaCl pH 8, 4% acetonitrile. UV detection is performed at 280 nm. The chromatography profile is illustrated in FIG. 7B.

TABLE 9
Results of Ion Exchange Chromatography analyses Example 7B

| | Total acidic | % Main | Total basic |
|---|---|---|---|
| Sample | variants | peak | variants |
| Pembrolizumab | 17.5 | 62.7 | 19.8 |
| 50 mM Hepes, pH 7.0 | | | |
| 225 mg/ml | | | |
| (starting material) | | | |
| Crystalline suspension | 15.1 | 65.1 | 19.8 |
| Example 7B | | | |
| 209 mg/ml | | | |
| (final) | | | |

### Example 8

### Example 8A: Batch Crystallization of Pembrolizumab at 206 mg/Ml at 30 ml Scale

30 grams of pembrolizumab at 206 mg/ml in 10 mM histidine, 10 mM methionine, pH 5.4 was added into a 50 mL glass reactor (part #: 51161838, METTLER TOLEDO, Columbus, OH). The reactor was then set up on the Easy Max 102 LT Advanced Synthesis Workstation (part #: 30548000, METTLER TOLEDO, Columbus, OH). The reactor stir was set to agitate at 50 rpm and a temperature of 22° C. 2.2 mL of 25 mg/ml caffeine (part #: C0750-500G, Sigma-Aldrich, St. Louis, MO) solution in 150 mM Hepes, pH 7.0 was slowly added into the reactor. The reactor was aged for 24 hours. The mixture started to turn into a visually white/opaque suspension from the top surface in about 2 hours. The mixture appeared to be a homogeneous suspension after 24 hours of aging.

The pH of the suspension was measured 6.3 by the SevenGo Duo pH/Ion/DO meter SG68 (part #: 51302610, METTLER TOLEDO, Columbus, OH). The suspension was confirmed to be crystalline by SONICC and ssNMR. The quality of the crystalline suspension was evaluated by UPSEC, IEX, NR-CE-SDS and HIC, and all shown to be comparable.

### Example 8B: SONICC Characterization of Crystalline Pembrolizumab Suspension Derived from Example 8A

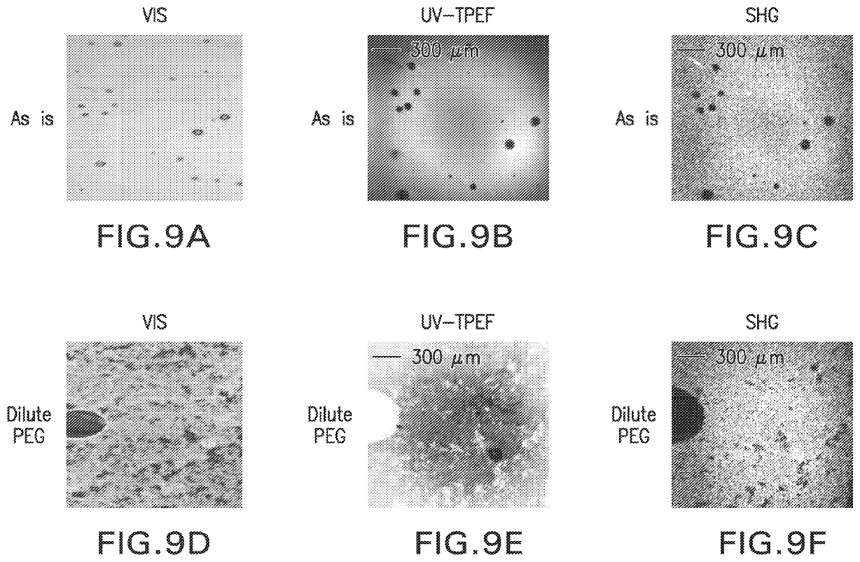

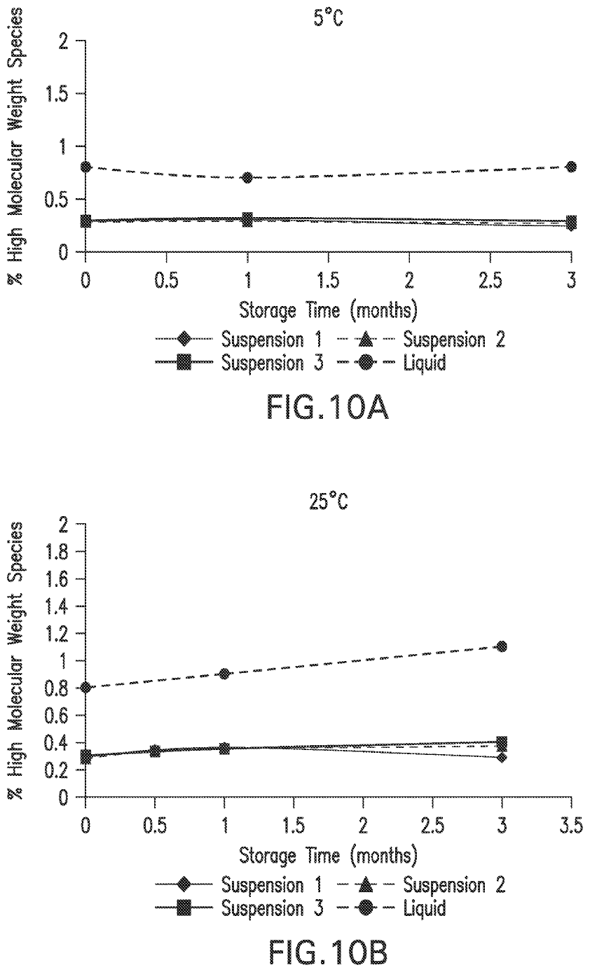

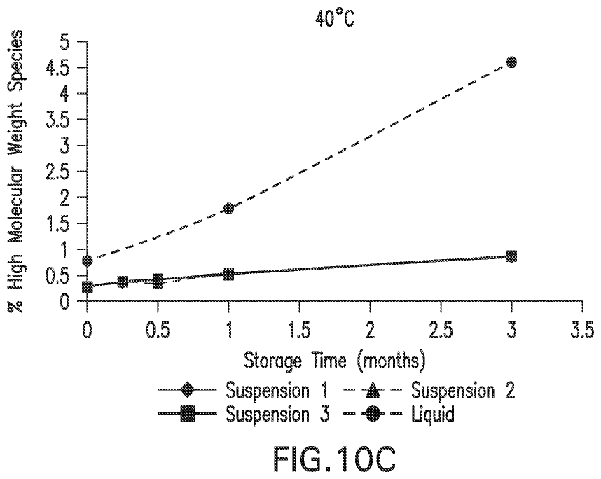

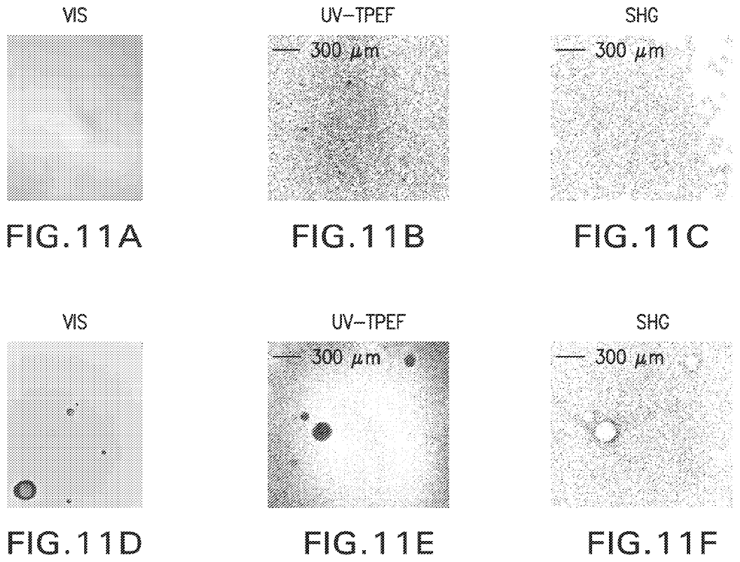

2 μl of the suspension from Example 8A was dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW)) settings shown in Example 8A. The upper and lower panels were UV+ and SHG+ consistent with a chiral crystalline suspension. Results shown in FIG. 9.

### Example 8C: Size Exclusion Chromatography Multi-Angle Light Scattering Analyses of Redissolved Crystalline Pembrolizumab Suspension Derived from Example 8A

Size Exclusion Chromatography Multi-Angle Light Scattering (SEC-MALS) is utilized to determine the molecular weight (MW). This method utilizes the Waters Acquity UPLC H-Class Bio system (attached to a Wyatt UP LS laser and UP tRex detector) with a Waters Acquity BEH200 SEC column to separate molecules based on their hydrodynamic radius. Resolved peaks are detected by the absorbance at 280 nm, light scattering intensity, and dRI (differential Refractive Index). This method is run under isocratic conditions at a flow rate (FR) of 0.5 mL/min in a buffer composed of 50 mM phosphate, 450 mM arginine, pH 7.0. SEC-MALS results quantify monomer ≥99.0%; HMW≤1.0%.

### Example 8D: Formulation Screening Studies of Crystalline Pembrolizumab Suspension Derived from Example 8A

A crystalline slurry containing ~190 mg/mL pembrolizumab, 10 mM Hepes, 9 mM L-methionine, 9 mM histidine, and 1.7 mg/mL caffeine from Example 8A was formulated using stock solutions of excipients as three different formulations. The slurry was added to a 15 ml conical tube, the excipient stock solutions were added, and then the formulation was mixed manually with a metal spatula and then by inverting the tube. The formulations are shown in Table 10.

TABLE 10
Composition of Formulations made from
Crystalline suspension Example 8A
Formulating from Slurry

| | Starting DS | Suspension Formulation | | |
|---|---|---|---|---|
| Component | (Slurry) | 1 | 2 | 3 |
| Pembrolizumab (mg/mL) | ~190 | 130 | 130 | 130 |
| HEPES (mM) | 10 | 20 | 20 | 20 |
| PEG 3350 (mg/mL) | 0 | 10 | 10 | 0 |
| L-Arginine HCl (mM) | 0 | 50 | 100 | 100 |
| NaCl (mM) | 0 | 50 | 0 | 0 |
| L-Methionine | 9 | 12 | 12 | 12 |
| Histidine (mM) | 9 | 7 | 7 | 7 |
| Caffeine (mg/mL) | 1.7 | 1.3 | 1.3 | 1.3 |

The formulations were filled into 2R glass vials with a 0.5 mL fill volume, stoppered using 13 mm grey chlorobutyl stoppers, and sealed with an aluminum flip-off cap. These formulations were staged at 5° C., 25° C./60% RH, and 40° C./75% RH. One vial was staged at each condition.

### SONICC:

A Formulatrix SONICC imaging system was used to assess whether protein crystals were present in the formulations. Approximately 20 μL of each formulation was pipetted onto a glass slide and then a spherical coverslip was added over top of the slide and sample. The slides were mounted in the SONICC imager. Imaging parameters were as follows: visible images were taken at 5 MP, ultraviolet two photon excited fluorescence (UV-TEPF) were taken using the standard settings, and the second harmonic generation (SHG) images were taken at 450 MW and/or at 400 mW with 4000 ms exposure.

### Osmolality:

An Advanced Instrument Osmometer (Model 3250) was used to measure the osmolality of the formulations by freezing-point depression. Three different osmolality standards were run (100 mOsm/kg, 290 mOsm/kg, and 850 mOsm/kg) and all passed their tolerance to ensure the instrument was in working order. Each sample was pipetted into the sample tube (250 μL), and then placed in the instrument and the measurement was started. Viscosity:

The viscosity of the formulations was measured using a RheoSense Inc m-VROC instrument. The sample was loaded into a 1 mL Hamilton glass syringe and then placed into the equipment. The operating parameters for the instrument were: 20° C. temperature, 4000 1/s shear rate, 3 s hold time, and 5.1 s measuring time. The chip used had a 50.2-micron flow channel depth.

TABLE 11
Osmolality and Viscosity Results for Three
Suspensions described in Example 8D

| | Osmolality | Viscosity |
|---|---|---|
| Sample | (mOsm/kg) | (cP) |
| Suspension 1 | 291 | 9.4 |
| Suspension 2 | 295 | 14.4 |
| Suspension 3 | 287 | 9.4 |

### Example 9

### Example 9A: Preparation of Crystalline Pembrolizumab Drug Substance 1

30 grams of pembrolizumab at 206 mg/ml in 10 mM histidine, 10 mM Methionine, pH 5.4 was added into a 50 mL glass reactor (part #: 51161838, METTLER TOLEDO, Columbus, OH). The reactor was then set up on the Easy Max 102 LT Advanced Synthesis Workstation (part #: 30548000, METTLER TOLEDO, Columbus, OH). The reactor stir was set to agitate at 50 rpm and temperature was set at 22° C. 2.2 mL of 25 mg/ml) caffeine (part #: C0750-500G, Sigma-Aldrich, St. Louis, MO) solution in 150 mM HEPES, pH 7.0 was slowly added into the reactor. The reactor was aged for 24 hours. The mixture started to turn into a visually white/opaque suspension from the top surface in about 2 hours. The mixture appeared to be a homogeneous suspension after 24 hours of aging.

The pH of the suspension was measured 6.3 by the SevenGo Duo pH/Ion/DO meter SG68 (part #: 51302610, METTLER TOLEDO, Columbus, OH). The suspension was confirmed to be crystalline by SONICC and ssNMR. The quality of the crystalline pembrolizumab suspension drug substance 1 was evaluated by UPSEC, IEX, NR-CE-SDS and HIC, and all shown to be be comparable.

### Example 9B: Preparation of Crystalline Pembrolizumab Drug Product 1 (165 mg/Ml)

The preparation of crystalline pembrolizumab drug product (165 mg/ml) was undertaken as per Example 11.

### Example 9C: SONICC Characterization of Crystalline Pembrolizumab Drug Substance 1 at (206 mg/Ml at 30 ml) Scale and Crystalline Pembrolizumab Drug Product 1

2 μl of the crystalline pembrolizumab drug substance 1 and crystalline pembrolizumab drug product 1 from Example 9a and 9b respectively were dispersed in 18 μl of 10 mM Hepes, pH 6.8, 8% PEG 3350 buffer and placed within a well of a 12-sample slide platform and analyzed using SONICC analyses (visible; (5 mega pixels), UV-TFEF (standard) and SHG (450 mW) settings. The upper and lower panels were UV+ and SHG+ consistent with a chiral crystalline suspension. Results shown in FIG. 11.

### Example 9C: Size Exclusion Chromatography (SEC) Multi-Angel Light Scattering Analyses of Redissolved Crystalline Pembrolizumab Drug Product 1 Derived from Example 9B

Size Exclusion Chromatography Multi-Angle Light Scattering (SEC-MALS) is utilized to determine the molecular weight (MW). This method utilizes the Waters Acquity UPLC H-Class Bio system (attached to a Wyatt UP LS laser and UP tRex detector) with a Waters Acquity BEH200 SEC column to separate molecules based on their hydrodynamic radius. Resolved peaks are detected by the absorbance at 280 nm, light scattering intensity, and dRI (differential Refractive Index). This method is run under isocratic conditions at a flow rate (FR) of 0.5 mL/min in a buffer composed of 50 mM phosphate, 450 mM arginine, pH 7.0. SEC-MALS results quantify monomer ≥99.0%; HMW≤1.0%.

### Example 9D: Viscosity Characterization of Crystalline Pembrolizumab Drug Product 1 (DP1) Derived from Example 9B

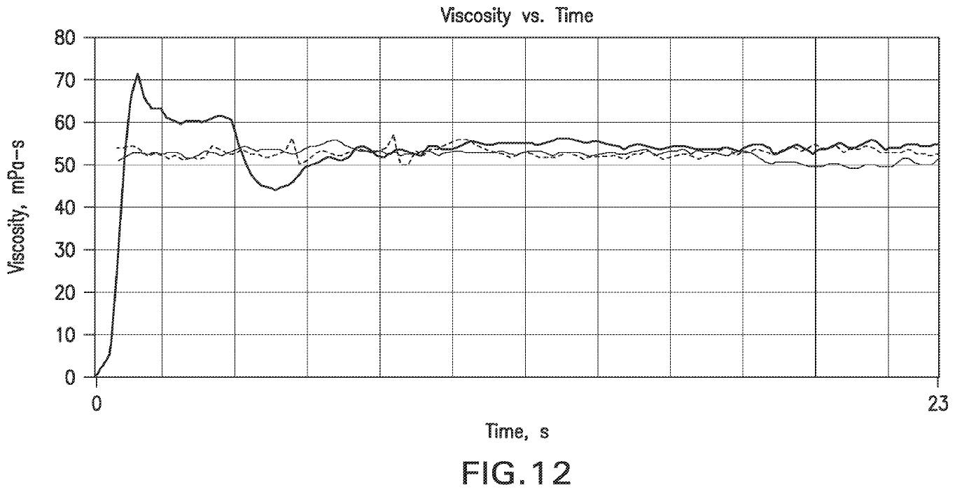

A Rheosense m-VROC instrument from RheoSense Inc., San Ramon CA derives viscosity from pressure drop using Hagen-Poiseuille equation. The viscosity of drug product 1 (165 mg/mL) crystalline suspension formulation was measured using a shear rate of 1000 1/s. The average viscosity of the anti-PD-1 crystalline suspension formulation drug product 1 derived from Example 9 at 20° C. was 53 cP. The viscosity vs. time plot is shown in FIG. 12.

### Example 9E: Injectability Studies of Crystalline Pembrolizumab Drug Product 1 (DP1) Derived from Example 9B

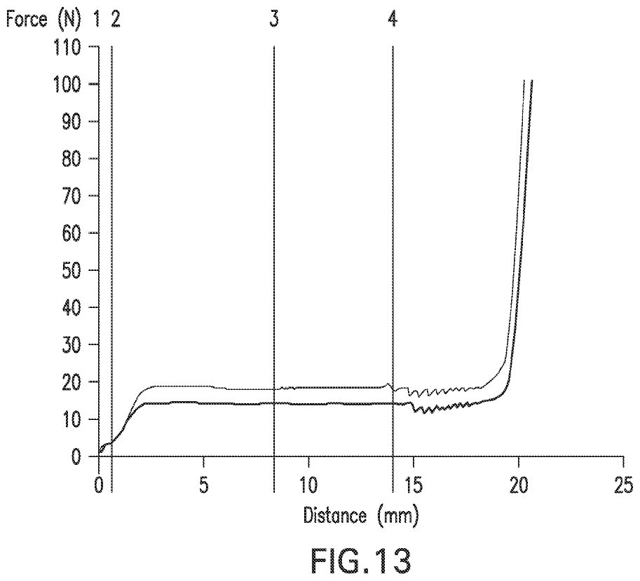

A TA XT Plus Texture Analyzer was used to measure break-loose force and glide-force. The Nemera plunger rod was screwed into the pre-filled syringe stopper and the needle shield was removed. The pre-filled syringe with plunger rod was inserted into the machine and the test was run. The formulation was expelled into a glass beaker and the total test time was 12 seconds. Two pre-filled syringes were used for measurement. The mean dose delivered was 1.07 g with a break loose of 3.63N and a glide force of 16.47 N. The results are shown in FIG. 13.

TABLE 10
Results of the two pre-filled syringe samples
for break-loose force and glide-force.

| | Dose | | Break | Glide |
|---|---|---|---|---|
| Sample # | Delivered (g) | | Loose (N) | Force (N) |
| 1 | 1.0506 | | 3.497 | 18.65 |
| 2 | 1.0667 | | 3.758 | 14.29 |
| Mean | 1.05865 | | 3.6275 | 16.47 |
| Sdtev | 0.011384419 | | 0.18455487 | 3.082985566 |
| Density: | 1.05564 | g/cm³ | | |
| Dose Delivered: | 1.003 | mL | | |

### Example 9F: SCISSOR Characterization Pembrolizumab Liquid Vs. Crystalline Pembrolizumab Drug Product 1 Derived from Example 9B

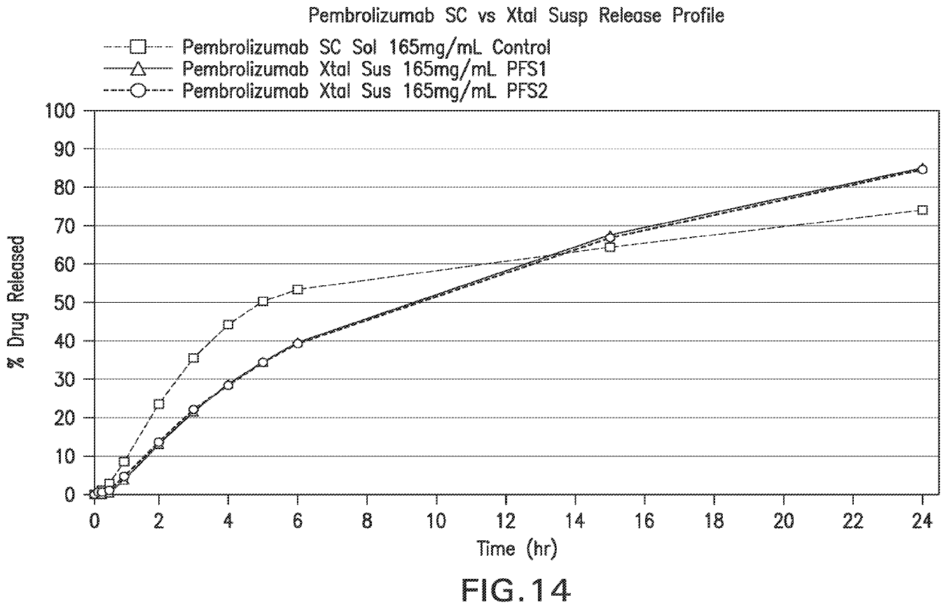

Crystalline pembrolizumab drug product 1 derived from Example 9B was evaluated and compared to a pembrolizumab substance both at 165 mg/ml on Subcutaneous Injection Site SimulatOR (SCISSOR), Sirius Analytical Ltd (now known as Pion, Forest Row, UK). The experiments were performed according to the Pion's SCISSOR manual recommended conditions as reported in Kinnunen Bown et al., Journal of Control Release, 2018. A 1 mL disposable syringe (BD 1 mL TB Syringe, Sterile, 25G×⅝ (0.5 mm×16 mm), REF 309626), filled 0.85 mL of each formulation and was injected (with approximately 1 min injection time) into the commercially available SCISSOR cartridge (Standard 5 μm pore size) filled with extracellular matrix (ECM, Pion, SCISSOR Cartridge Pack, P/N 11401-0001, Lot #SECM0121) which had been allowed to equilibrate at 34° C. in the surrounding “infinite sink” chamber conditions. The chamber was filled with 300 mL of physiological, carbonate based buffer solution, 1 L of which contains 6.4 g NaCl, 0.09 g MgCl₂ hexahydrate, 0.4 g KCl, 0.2 g CaCl₂ dihydrate, 2.1 g NaHCO₃, and 0.2 g of NaN₃ (to prevent bacterial growth) dissolved in Milli-Q water, maintained at 34° C. and pH 7.4 by bubbling CO₂ (gas) through the buffer. Aliquots of 430-450 μL volumes, depending on the time point, were taken from the “infinite sink” buffer chamber at 0, 5, 10, 20, 30 min, 1, 2, 3, 4, 5, 6, 15, and 24 hrs. after injection, utilizing Gilson autosampler, to be subjected to quantitative analysis on the absorbance plate reader (200 μL (n=2), LM₁=285 nm LM₂=320 nm, Wavelength Comb=LM₁-LM₂, SpectraMax M5, Molecular Devices) to acquire the release profile for each formulation. The release profiles overlays of the crystalline and liquid formulations are shown in FIG. 14.

### Example 9G: Particle Size Analyses of Crystalline Pembrolizumab Drug Substance 1 and Crystalline Pembrolizumab Drug Product 1 Derived from Example 9A and 9B

Instrument: Malvern Mastersizer 3000 with a Hydro MV unit and stir rate of 1800 rpm. Particle refractive index was set to 1.54 and Fluid index set to 1.33. Results are shown in Table 11.

TABLE 11
Particle Size Analyses of crystalline pembrolizumab
drug substance 1 and crystalline pembrolizumab
drug product 1 derived from Example 9a and b

| Sample | D10 (μm) | D50 (μm) | D90 (μm) |
|---|---|---|---|
| Drug substance 1 | 0.5 | 1.3 | 5.2 |
| Drug product 1 | 0.3 | 0.5 | 1.1 |

### Example 10

Formulation and Stability of Crystalline Suspension at 130 mg/Ml

A starting drug substance (slurry) comprising about 190 mg/ml pembrolizumab, 10 mM Hepes, 9 mM L-methionine, 9 mM histidine, and 1.7 mg/mL caffeine was formulated into three suspensions shown in the following Table. The suspensions were then stored at 5° C., 25° C. and 40° C. up to 3.5 months.

TABLE 12
Formulating suspensions from starting slurry

| | Starting | Final drug product | | |
|---|---|---|---|---|
| Component | DS | 1 | 2 | 3 |
| Pembrolizumab (mg/ml) | ~190 | 130 | 130 | 130 |
| HEPES (mM) | 10 | 20 | 20 | 20 |
| PEG 3350 (mg/mL) | 0 | 10 | 10 | 0 |
| L-Arginine HCl (mM) | 0 | 50 | 100 | 100 |
| NaCl | 0 | 50 | 0 | 0 |
| L-Methionine | ~9 | ~12 | ~12 | ~12 |
| Histidine (mM) | ~9 | ~7 | ~7 | ~7 |
| Caffeine (mg/mL) | 1.7 | 1.3 | 1.3 | 1.3 |

Stability results from Example 10 indicate that all three suspensions are stable at the three timepoints. Osmolality and viscosity readings of the three suspensions at initial timepoint and subsequent timepoints all showed comparable results.

TABLE 13
Osmolality and initial viscosity measurements
for three suspensions from Ex. 10

| | Osmolality | Viscosity |
|---|---|---|
| Sample | (mOsm/kg) | (cP) |
| Suspension 1 | 291 | 9.4 |
| Suspension 2 | 295 | 14.4 |
| Suspension 3 | 287 | 9.4 |

### Example 11

Preparation of Crystalline Pembrolizumab Drug Product Suspension at 165 mg/Ml

6.5 g of 0.2 μm filtered pembrolizumab drug substance in an appropriate glass container maintained at 22° C. was charged with 4.5 mL of 0.2 μm filtered caffeine solution. The solution was slowly mixed and the batch was incubated at 22° C. overnight. The incubation temperature increased from 22° C. to 28° C. HEPES buffer (pH 7.2) from a 1M stock was added to the solution to a afford a final volume of 32.71 mL. A charge of 0.5 mL of L-methionine stock (20 mg/mL), 0.80 mL PEG3350 from a stock of 400 mg/mL of 10 mg/mL, 0.86 mL of L-arginine from a stock of 400 mg/mL, 0.478 mL of sodium chloride from a stock of 200 mg/mL to a final concentration of 2.92 mg/mL was added to the solution. Finally, water is added to a final volume of 32.54 mL to obtain pembrolizumab crystalline suspension with final composition as provided in the table below.

TABLE 14
Final composition of crystalline suspension
listed in Example 11, pH 6.0

| Component | Suspension 1 |
|---|---|
| pembrolizumab (mg/mL) | 165 |
| HEPES (mM) | 20 |
| PEG 3350 (mg/mL) | 10 |
| L-Arginine HCl (mM) | 50 |
| NaCl (mM) | 50 |
| L-Methionine | 10 |
| Histidine (mM) | ~7 |
| Caffeine (mg/mL) | 1.3 |

### Example 12

### Comparison of Different Crystallization Conditions and Impact on Impurities

Pembrolizumab crystals were obtained from two different crystallization conditions consisting of either i) ammonium phosphate, or ii) PEG-3350 and caffeine.

From initial screening experiments of pembrolizumab (45 mg/ml in 10 mM histidine buffer, pH 6.0) using a high-throughput screening and discovery crystallization strategy two conditions were identified that led to its crystallization. One condition consisted of 8-10% PEG-3350 in 20 mM HEPES, pH 6.8 at RT. The other condition consisted of ammonium phosphate in Tris buffer-1.8 M NH₄H2PO₄ and 100 mM Tris, pH 8.0. T=30° C. All experiments with ammonium phosphate were carried out in 1.8-2.2 M final concentration of ammonium phosphate at 30° C. for 24 hours to achieve >95% crystallization yield. Whereas, experiments with PEG-3350 and caffeine were carried out at RT for 4 hrs to reach reaction completion. After the completion of the crystallization reaction, samples of crystallization slurry were spun down at 6000 rpm in a microcentrifuge and the supernatant collected. The pellet of protein crystals was further washed in a buffer consisting of the original reaction components without the mAb. After two rounds of centrifugation and wash/supernatant removal, the protein pellet was dissolved in 10 mM Histidine, pH 5.5 buffer and analyzed for residual host cell proteins (HCP), and residual DNA. Product quality and impurity profile were also analyzed via size-exclusion chromatography. Residual host-cell protein (HCP) analysis was carried out using commercially available Chinese Hamster Ovary (CHO)-HCP Elisa Kit from Cygnus Technologies. Residual CHO DNA analysis was carried out using a commercial-available qPCR-based assay kit.

Pembrolizumab crystallization was also carried out using different feed types that had varying levels of residual impurities. These feeds included i) Protein A Pool (PAP), ii) filtered, neutralized, viral-inactivated pool (FNVIP), and iii) AEX Pool (AEXP). Pembrolizumab crystals were obtained using all the three pools and the two crystallization conditions. The components of the feed did not appear to impact the crystallization outcome and yields.

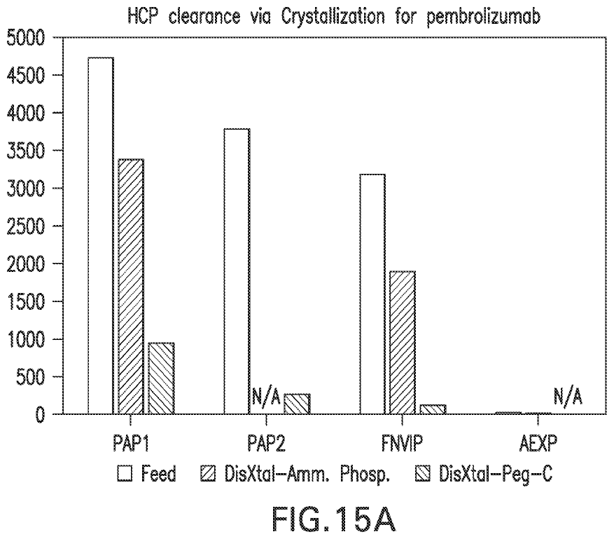

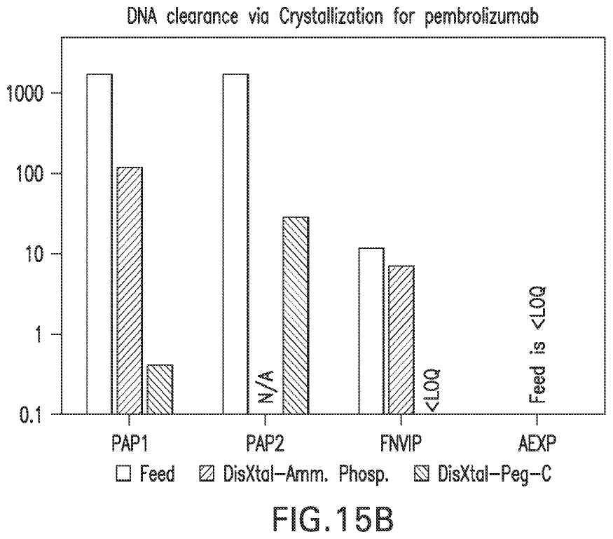

A difference was observed between the two crystallization conditions in terms of clearance of impurities during crystallization and re-dissolution of pembrolizumab crystals (see FIGS. 15A and 15B). Crystallization using the PEG-Caffeine condition offers better clearance of residual HCP/DNA/ProA and HMW species in the PAP and FNVIP samples. The PEG-caffeine condition showed much higher clearance of both residual HCP and DNA impurities from all types of intermediate pools as compared to the Ammonium Phosphate condition. Less than a 2-fold clearance of residual HCP and DNA impurities was obtained under the ammonium phosphate condition. Differential scanning calorimetry (DSC) results showed reduced thermal stability in the presence of ammonium phosphate. It is likely that ammonium phosphate-mediated partial unfolding or destabilization of pembrolizumab leads to exposure of hydrophobic patches that result in HCPs interacting with the mAb and being a part of the crystal lattice or present as a layer on the crystal surface that does not get washed off in the subsequent wash steps. It is also likely that ammonium phosphate also leads to the denaturation, aggregation and subsequent irreversible association of HCPs and DNAs with the pembrolizumab crystals, thereby leading to poor clearance. These results further demonstrate that at not all crystallization conditions are amenable for a bioprocessing application especially, for protein purification.

A nuanced and biophysical approach is needed to design a process for purification, starting with dirtier feed streams such as HCCF, PAP or FNVIP. Impurities need to be examined in parallel to crystallization conditions in order to purify and preserve the mAb structure while excluding impurities during crystallization, washes and filtration.

### Example 13

### Crystalline Phase Identification and Characterization of Pembrolizumab Crystalline Suspensions Using SAXS and Solid-State NMR

As those of ordinary skill in the art readily appreciate, the physical characteristics of a crystal may be effectively characterized by its diffraction peaks obtained from x-ray diffraction or x-ray scattering such as Small Angle X-ray Scattering (SAXS). The physical characteristic of a crystal may also be characterized by spectroscopic techniques such as ¹³C solid-state NMR analysis. Such combination of characterizations is effectively used to identify and distinguish distinct crystalline phases of mAb crystals. In particular, SAXS and solid-state NMR analysis are used to identify and distinguish two distinct crystalline phases of pembrolizumab, namely Phase 1 and Phase 2, prepared under Caffeine crystallization conditions.

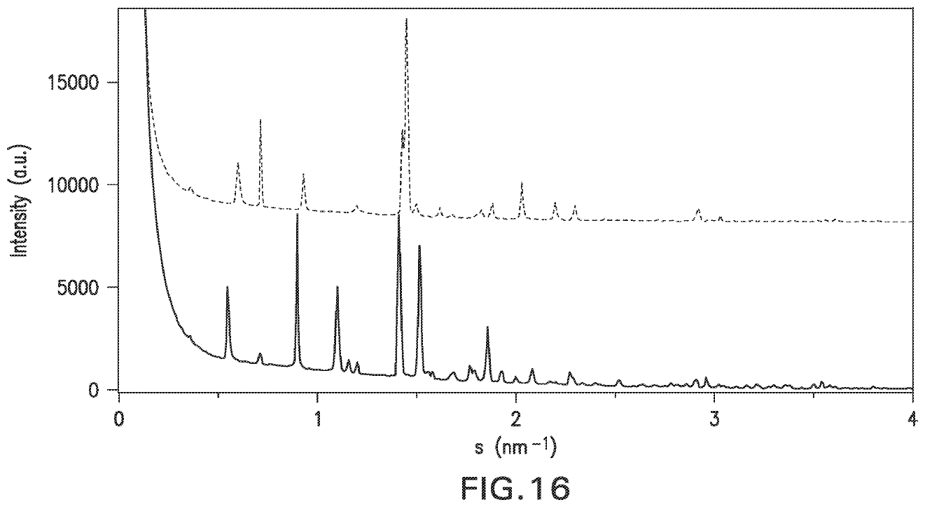

FIG. 16 and shows the synchrotron SAXS patterns of crystalline Phase 1 (solid line) and crystalline Phase 2 (dashed line).

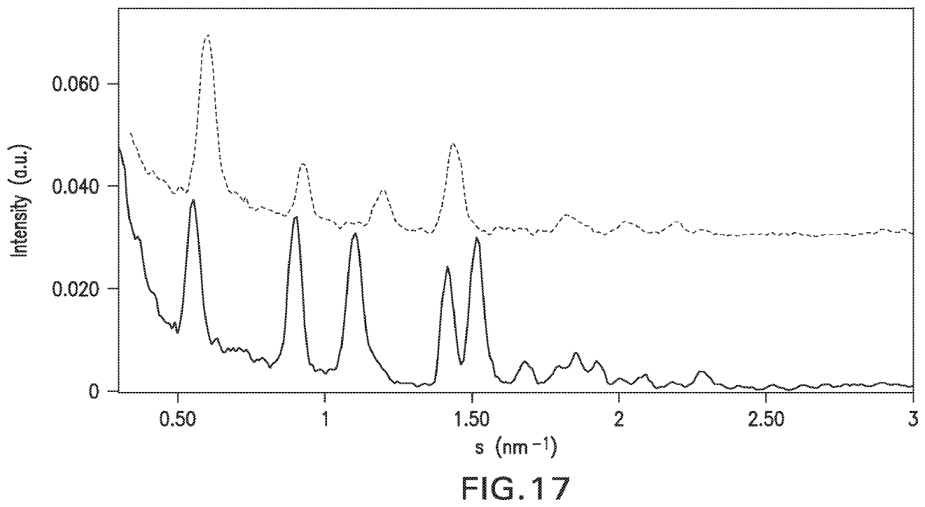

FIG. 17 displays the laboratory SAXS patterns of crystalline Phase 1 (solid line) and crystalline Phase 2 (dashed line).

Synchrotron SAXS analysis were performed on the PETRA diffractometer at the Deutsches Elektronen Synchrotron (DESY) facility in Hamburg, Germany. The diffractometer was equipped with a Pilatus 6M detector and operated at a current of 100 A. The analysis were performed in the range of 0.029 nm-1 to 7.432 nm-lin angular momentum(s) and with a step size of 0.028 nm-1 and exposure times of 0.1s/step. Clear crystallization liquors (free of crystals) were used as background and subtracted from the crystalline suspensions data.

Laboratory SAXS analysis were performed on a Xenocs Xeuss 3.0 diffractometer equipped with a High Flux Very Long Focus (HFVL) GeniX 3D Cu K alpha x-ray generator and a two-dimensional EIGER2 R 1M vacuum-compatible detector distant from 0.37 m away to the sample. The analysis were performed in the range of 0.128 nm-1 to 7.199 nm-lin angular momentum(s) with a step size of 0.082 nm-1 and a total data acquisition of 40 min. Clear crystallization liquors (free of crystals) were used as background and subtracted from the crystalline suspensions data.

Those skilled in the art will also recognize that SAXS acquired with a synchrotron beamline (referred herein to as synchrotron SAXS) results in diffraction patterns displaying enhanced resolution as compared to a laboratory SAXS equipment (referred herein to as laboratory SAXS) due to higher radiation's energy and smaller beam size. Synchrotron SAXS patterns therefore display more diffraction peaks with finer structures as compared to laboratory SAXS patterns. Those skilled in the art will recognize that the measurements of the SAXS peak locations for a given crystalline form of the same compound will vary within a margin of error. Variability can depend on such factors as the system, methodology, sample, and conditions used for measurement. As will also be appreciated by the skilled crystallographer, the intensities of the various peaks reported in the figures herein may vary due to several factors such as orientation effects of crystals in the x-ray beam, the purity of the material being analyzed, and/or the degree of crystallinity of the sample. The skilled crystallographer also will appreciate that measurements using a different wavelength will result in different shifts. Such further SAXS patterns generated by use of alternative wavelengths are alternative representations of the SAXS patterns of the crystalline material of the present invention and as such are within the scope of the present invention. The intensities of the peaks (y-axis is in counts) were plotted versus the momentum transfer (or s) (x-axis is in nm⁻¹).

Peak locations (on the momentum transfer x-axis) consistent with synchrotron SAXS profiles are displayed in Table A. Table A displays the locations of the synchrotron SAXS peaks characteristic of Phase 1 in the first and second column. Table A displays the locations of the synchrotron SAXS peaks characteristic of Phase 2 in the third and fourth columns.

TABLE A
Synchrotron SAXS diffraction peaks for
experimental Phase 1 and Phase 2.

| | |
|---|---|
| Synchrotron SAXS | Synchrotron SAXS |
| diffraction peaks for | peaks characteristic |
| experimental Phase 1 | of Phase 2. |

| Pos. [nm⁻¹] | Rel. Int. [%] | Pos. [nm⁻¹] | Rel. Int. [%] |
|---|---|---|---|
| 0.55 | 58.74 | 0.36 | 17.24 |
| 0.71 | 20.47 | 0.60 | 22.59 |
| 0.90 | 100.00 | 0.71 | 50.88 |
| 1.10 | 58.36 | 0.93 | 23.65 |
| 1.16 | 17.14 | 1.20 | 8.02 |
| 1.20 | 15.73 | 1.42 | 24.87 |
| 1.41 | 99.41 | 1.45 | 100.00 |
| 1.51 | 82.10 | 1.50 | 9.01 |
| 1.55 | 10.08 | 1.62 | 7.21 |
| 1.58 | 9.17 | 1.68 | 3.88 |
| 1.69 | 9.68 | 1.79 | 2.96 |
| 1.76 | 13.72 | 1.82 | 4.87 |
| 1.79 | 10.80 | 1.88 | 9.43 |
| 1.86 | 20.94 | 1.99 | 2.34 |
| 1.93 | 10.96 | 2.02 | 9.95 |
| 2.00 | 7.17 | 2.06 | 2.60 |
| 2.08 | 11.64 | 2.18 | 2.02 |
| 2.17 | 4.87 | 2.19 | 5.60 |
| 2.20 | 4.34 | 2.30 | 8.88 |
| 2.27 | 9.84 | 2.44 | 1.62 |
| 2.34 | 3.40 | 2.50 | 1.13 |
| 2.40 | 3.44 | 2.58 | 1.14 |
| 2.42 | 2.80 | 2.72 | 1.49 |
| 2.52 | 5.47 | 2.79 | 0.95 |
| 2.63 | 2.57 | 2.86 | 0.92 |
| 2.70 | 2.64 | 2.88 | 1.21 |
| 2.72 | 1.60 | 2.92 | 7.51 |
| 2.74 | 1.99 | 2.98 | 1.30 |
| 2.78 | 3.60 | 3.03 | 3.59 |
| 2.81 | 2.71 | 3.09 | 1.04 |
| 2.86 | 3.03 | | |
| 2.91 | 4.24 | | |
| 2.95 | 6.75 | | |

Peak locations (on the momentum transfer x-axis) consistent with laboratory SAXS profiles are displayed in Table B. Table B 1^{st} and 2^{nd} columns displays the locations of the laboratory SAXS peaks characteristic of Phase 1. Table B 3^{rd} and 4^{th} columns displays the locations of the laboratory SAXS peaks characteristic of Phase 2.

TABLE B
Laboratory SAXS diffraction peaks for
experimental Phase 1 and Phase 2.

| | |
|---|---|
| Laboratory SAXS | Laboratory SAXS |
| diffraction peaks for | diffraction peaks for |
| experimental Phase 1 | experimental Phase 2 |

| Pos. [nm⁻¹] | Rel. Int. [%] | Pos. [nm⁻¹] | Rel. Int. [%] |
|---|---|---|---|
| 0.55 | 100.00 | 0.60 | 100.00 |
| 0.90 | 91.23 | 0.93 | 35.71 |
| 1.10 | 81.92 | 1.20 | 22.45 |
| 1.41 | 58.36 | 1.44 | 43.88 |
| 1.52 | 79.45 | 1.81 | 9.18 |
| 1.69 | 10.68 | 2.02 | 6.12 |
| 1.84 | 15.34 | 2.20 | 6.12 |
| 1.93 | 11.51 | | |
| 2.28 | 7.67 | | |

Pembrolizumab crystalline suspensions were identified and distinguished from one another by solid-state NMR. Solid-state NMR spectra were acquired on a Bruker Avance III 500 MHz spectrometer equipped with a 4.0 mm H/C/N MAS probe for ¹³C (carbon-13) cross polarization (CP) magic angle spinning (MAS) experiments. One-dimensional (1D) 1H-¹³C CP MAS spectra were collected at 10° C. with a CP contact time of 1.5 ms, a recycle delay of 2.5 s, 1H SPINAL-64 decoupling at 83 kHz strength, and MAS frequency of 12.0 kHz. The ¹³C chemical shifts were externally referenced to the carboxyl peak of α-glycine at 176.49 ppm. All NMR spectra were processed using Bruker TopSpin. For purposes of solid-state NMR, the term “about” mean±0.1 ppm.

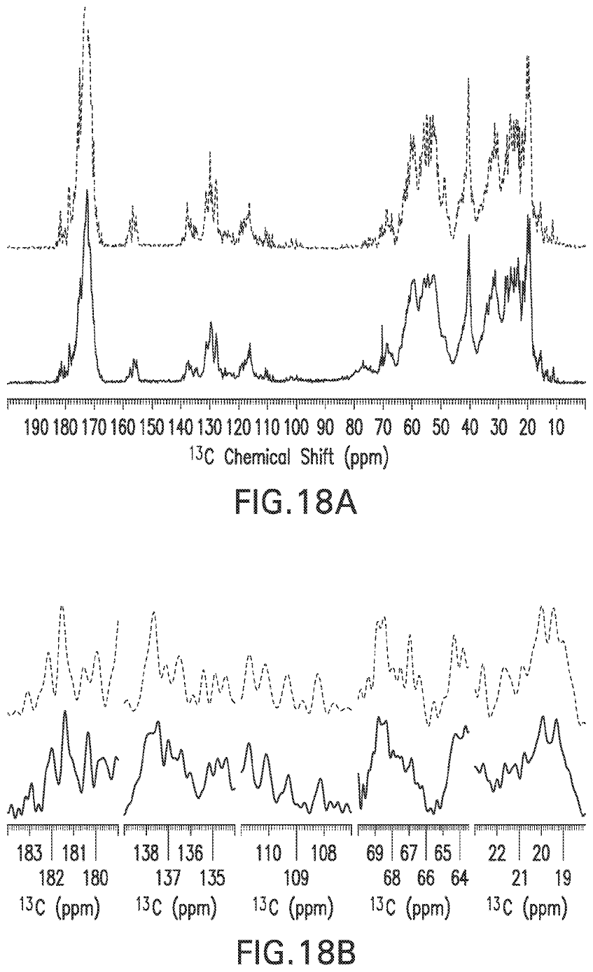

The pembrolizumab crystalline suspension samples were measured using the 500 MHz solid-state NMR equipment and procedures described above. The ¹³C (carbon-13) CP MAS NMR spectra for the crystals of pembrolizumab crystalline phase 1 and of crystals of pembrolizumab crystalline Phase 2 were acquired. The full spectra of the two forms and a few enlarged regions are respectively shown in FIG. 18A. and 18B.

Characteristic solid-state NMR peaks for the pembrolizumab Phase 1 crystal were observed at about 181.90, 181.40, 180.36, 179,69, 137.00, 135.17, 109.28, 108.12, 66.88 and 65.35, 40.69, 27.66, 27.24, 20.75 ppm.

Characteristic solid-state NMR peaks for the pembrolizumab Phase 2 crystal were observed at about 182.16, 181.54, 180.59, 179,99, 137.16, 135.43, 109.36, 108.23, 66.97 and 65.57, 40.80, 27.50, 27.01, 20.85 ppm.

### Example 14

### Filtration and Washings of Pembrolizumab Crystalline Suspensions

Filtration and washing processing steps prior to drug isolation and storage are common processing steps for crystalline suspensions. Filtration is used often and proven to be an effective technique for isolating particles from their suspension state. Washing steps are important for purification of any impurities (solvents, additives, reactants, by-products, etc.) remaining in interstitial voids between solid particles and/or surface adsorbed. For mAb crystals, such processing steps easily dehydrate in water-poor environments and tend to collapse under repeated physical manipulations. Because filtration and washing are essential steps to ensure maximal product quality, the stability of pembrolizumab crystalline suspensions across these unit operations was investigated. SAXS analysis was leveraged to confirm crystallinity as compared to unprocessed crystalline suspensions.

Pembrolizumab crystalline suspensions were filtered and washed with process relevant liquids without alteration of crystallinity as confirmed by unchanged x-ray diffraction patterns from SAXS analysis. The wash liquids were process relevant aqueous pH buffer solutions prepared from, but not limited to, HEPES or TRIS, and optionally further comprising one or more of the initial crystallization components (precipitants, additives, excipients, etc.) including, but not limited to, PEG, caffeine, and DSS.

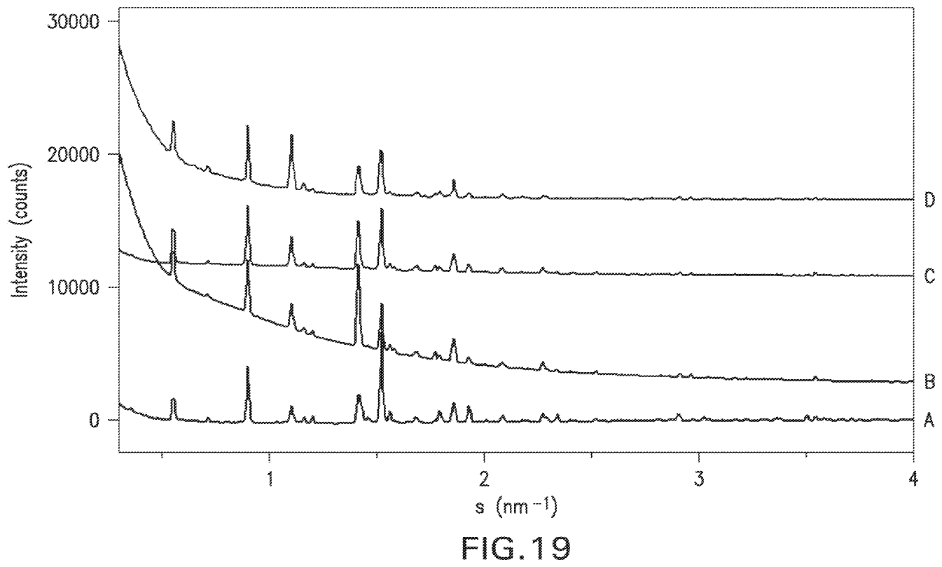

FIG. 19 shows synchrotron SAXS traces of a representative suspension of pembrolizumab crystalline phase 1: (A) as crystallized from an aqueous pH buffer solution containing L-histidine, caffeine, PEG3350 and DSS, (B) filtered and resuspended in an aqueous pH buffer solution containing PEG3350 and caffeine, (C) filtered and resuspended in an aqueous pH buffer solution containing caffeine and (D), filtered and resuspended in an aqueous pH buffer only. Crystallinity is maintained throughout as evidenced by the unchanged x-ray diffraction patterns.

### Example 14B: Selected Filter-Wash Protocols for Pembrolizumab Crystalline Phase 1

For Example 14B-D, a suspension of pembrolizumab crystalline phase 1 was prepared with a final pembrolizumab content of 18 mg/ml, a DSS content of 80 mg/ml, a PEG3350 content of 50 mg/ml, a caffeine content of 3 mg/ml solution and a HEPES content of 40 mM. The crystallized suspension displayed the SAXS pattern characteristic of the phase.

10 mL of a pembrolizumab crystalline phase 1 suspension were charged to a filter funnel. The particles were filtered by applying vacuum. The filtration was stopped when the liquid level was just above the level of the cake to avoid exposure of the particles to air.

A first wash solution was prepared by mixing 18 mL of water, 1.2 g of PEG3350, 0.6 mL of 1 M HEPES and 45 mg of caffeine. 10 mL of washing liquid was charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

A second wash solution was prepared by mixing 18 mL of water, 0.6 mL of 1 M HEPES and 45 mg of caffeine. 10 mL of washing liquid was charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

### Example 14C: Selected Filter-Wash Protocols for Pembrolizumab Crystalline Phase 1

10 mL of pembrolizumab crystalline phase 1 suspension were charged to a filter funnel. The particles were filtered by applying vacuum. The filtration was stopped when the liquid level was just above the level of the cake to avoid exposure of the particles to air. A wash solution was prepared by mixing 18 mL of water, 0.6 mL of 1 M HEPES and 48 mg of caffeine and was charged to the pre-filtered cake. The mixture was agitated to homogenize and aged over 20 min without agitation. After resuspension of the particles but prior to filtration, a sample of the suspension was withdrawn for SAXS analysis by applying vacuum until the liquid level was just above the level of the cake to avoid exposure of the particles to air.

### Example 14D: Selected Filter-Wash Protocols for Pembrolizumab Crystalline Phase 1

15 mL of pembrolizumab crystalline phase 1 suspension were charged to a filter funnel. The particles were filtered by applying vacuum. The filtration was stopped when the liquid level was just above the level of the cake to avoid exposure of the particles to air.

A first wash solution was prepared by mixing 36 mL of water, 2.4 g of PEG3350, 1.2 mL of 1 M HEPES and 90 mg of caffeine. 20 mL of washing liquid were charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

A second wash solution was prepared by mixing 36 mL of water, 1.2 mL of 1 M HEPES and 90 mg of caffeine. 20 mL of washing liquid were charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

A third wash solution was prepared by mixing 36 mL of water and 1.2 mL of 1 M HEPES. 20 mL of washing liquid were charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

### Example 14E: Selected Filter-Wash Protocols for Pembrolizumab Crystalline Phase 1

10 mL of a pembrolizumab crystalline phase 1 suspension were charged to a filter funnel. The particles were filtered by applying vacuum. The filtration was stopped when the liquid level was just above the level of the cake to avoid exposure of the particles to air.

A first wash solution was prepared by mixing 36 mL of water, 2.4 g of PEG3350, 1.2 mL of 1 M HEPES and 90 mg of caffeine. 10 mL of washing liquid were charged to the pre-filtered cake. The mixture was agitated to homogenize and aged over 30 min without agitation. After resuspension, the particles were filtered by applying vacuum until the liquid level was just above the level of the cake to avoid exposure of the particles to air.

A second wash solution was prepared by mixing 36 mL of water, 1.2 mL of 1 M HEPES and 90 mg of caffeine. 10 mL of washing liquid were charged to the pre-filtered cake. The mixture was agitated to homogenize and aged over 30 min without agitation. After resuspension, the particles were filtered by applying vacuum until the liquid level was just above the level of the cake to avoid exposure of the particles to air.

A third wash solution was prepared by mixing 36 mL of water and 1.2 mL of 1 M HEPES. 10 mL of washing liquid were charged to the pre-filtered cake. The mixture was agitated to homogenize and aged over 30 min without agitation. After resuspension, the particles were filtered by applying vacuum until the liquid level was just above the level of the cake to avoid exposure of the particles to air.

A fourth wash solution was prepared by mixing 36 mL of water and 90 mg of caffeine. 10 mL of washing liquid were charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

### Example 15

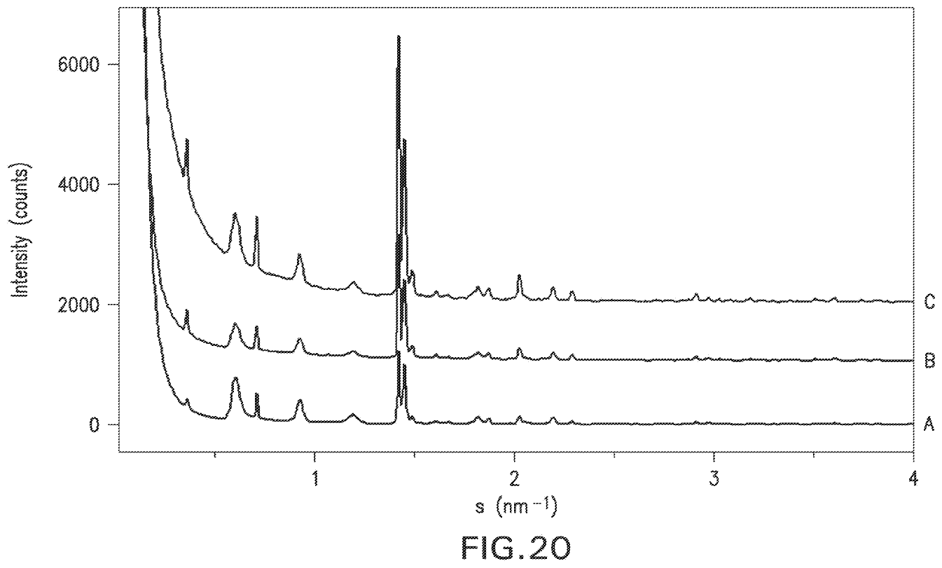

FIG. 20 shows synchrotron SAXS traces of a representative suspension of pembrolizumab crystalline phase 2: (A) as crystallized from an aqueous pH buffer containing L-histidine, caffeine and PEG3350, (B) filtered and resuspended in an aqueous pH buffer solution containing caffeine and (C), filtered and resuspended in an aqueous pH buffer solution only. Crystallinity is maintained throughout as evidenced by the unchanged x-ray diffraction patterns.

### Example 15A: Selected Filter-Wash Protocols for Pembrolizumab Crystalline Phase 2

Crystallization of pembrolizumab crystalline phase 2: a suspension of pembrolizumab crystalline phase 2 was prepared with a final pembrolizumab content of 14 mg/mL solution, a PEG3350 content of 60 mg/mL solution, a caffeine content of 2.3 mg/mL solution, a HEPES content of 40 mM and a L-Histidine content of 1 mM. The crystallized suspension displayed the SAXS pattern characteristic of the phase.

### Example 15B: Selected Filter-Wash Protocols for Pembrolizumab Crystalline Phase 2

10 mL of a pembrolizumab crystalline phase 2 suspension were charged to a filter funnel. The particles were filtered by applying vacuum. The filtration was stopped when the liquid level was just above the level of the cake to avoid exposure of the particles to air.

A first wash solution was prepared by mixing 18 mL of water, 0.6 mL of 1 M HEPES and 45 mg of caffeine and charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air. A second wash solution was prepared by mixing 18 mL of water with 0.6 mL of 1 M HEPES and charged to the pre-filtered cake. The mixture was agitated to homogenize and aged over 30 min without agitation. After resuspension of the particles but prior to filtration, a sample of the suspension was withdrawn for SAXS analysis by applying vacuum until the liquid level was just above the level of the cake to avoid exposure of the particles to air.

### Example 16

### Example 16A: Preparation of Crystalline Pembrolizumab Suspensions Through Semi-Continuous Batch Crystallizations

Two semi-continuous batch crystallizations were performed to assess the particles' filterability. The concentration of caffeine, PEG3350 and HEPES was the same in the two experiments. In the first experiment, the DSS level was kept constant throughout crystallization, while in the second DSS was only present in the seed bed. In both experiments, a crystalline suspension to be used as seed bed was prepared. 5 ml of pembrolizumab drug substance solution with a concentration of about 165 mg/ml were charged to a reactor equipped with an overhead stirrer. A solution was prepared in an appropriate separate container by dissolving 900 mg of DSS, 400 mg of PEG3350, 130 mg of caffeine in 13 ml water and 1.8 ml of HEPES buffer (pH 7.3) and was charged to the reactor. The mixture was aged under agitation over no less than 20 h after the point nucleation at 5° C. to produce a crystalline suspension.

In the first experiment, a solution was prepared in an appropriate container by dissolving 4500 mg of DSS, 2000 mg of PEG3350, 620 mg of caffeine in 64 ml water and 9.3 ml of HEPES buffer (pH 7.3). The solution was co-fed with 25 ml of pembrolizumab drug substance solution with a concentration of about 165 mg/ml to the previously prepared crystalline suspension over 20 h. During the addition, the content in the reactor was agitated and maintained at 5° C. After completion of the addition, the crystalline suspension was aged over no less than 1 h to further deplete supersaturation.

In the second experiment, a solution without DSS was prepared in an appropriate container by dissolving 2100 mg of PEG3350, 640 mg of caffeine in 64 ml water and 9.3 ml of HEPES buffer (pH 7.3). The solution was co-fed with 25 ml of pembrolizumab drug substance solution with a concentration of about 165 mg/ml to the previously prepared crystalline suspension over 20 h. During the addition, the content in the reactor was agitated and maintained at 5° C. After completion of the addition, the crystalline suspension was aged over no less than 1 h to further deplete supersaturation.

To allow for comparison an unseeded isothermal crystallization was performed. The overall composition was identical to the composition of the seed bed and of the first semi-continuous batch crystallization. 19 ml of pembrolizumab drug substance solution with a concentration of about 165 mg/ml was charged to a reactor equipped with an overhead stirrer. A solution was prepared in an appropriate separate container by dissolving 3100 mg of DSS, 1500 mg of PEG3350, 460 mg of caffeine in 48 ml water and 6.6 ml of HEPES buffer (pH 7.3) and was charged to reactor. The mixture was aged under agitation over no less than 48 h after the point nucleation at 5° C. to produce a crystalline suspension.

The three crystallizations afforded non-agglomerated, platelet-like particles. The semi-continuous batch crystallizations operations resulted in larger particles. The largest particles were obtained in the second semi-continuous batch operation, in which DSS was only present in the seed bed.

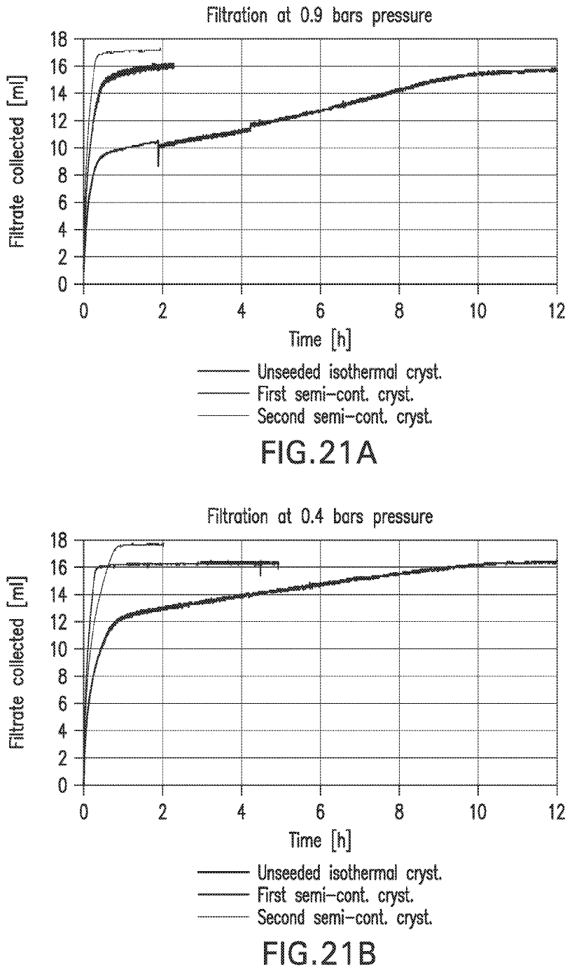

Approximately 20 ml of crystalline suspension were filtered on a pressure filter with an inner diameter of 35 mm with a mixed cellulose ester filter with a pore size 0.45 microns at 0.5 and 0.9 bars, respectively. FIGS. 21A and 21B show the collected filtrate over time.

As more filtrate was collected within a shorter period, it can be concluded that the semi-continuous batch operations resulted in an improved particles' filterability and in an improved deliquoring of the cake. For the unseeded crystallization, irrespective of the filtration pressure the filtrate flux slowed down significantly after having collected approximately 8 ml of filtrate resulting in excessively long filtration times.

### Example 16B: Preparation of Highly Agglomerated Crystals of Pembrolizumab to Ease Filtration

14 ml of pembrolizumab drug substance solution with a concentration of about 165 mg/ml was charged to a reactor equipped with an overhead stirrer. A solution was prepared in an appropriate separate container by dissolving 500 mg of DSS, 2000 mg of PEG3350, 350 mg of caffeine in 36 ml water and 5 ml of HEPES buffer (pH 7.3) and was charged to reactor. The composition of mixture was chosen to induce a liquid-liquid phase separation immediately after the charge of the solution. The mixture was aged under agitation over no less than 20 h at 5° C. to produce a crystalline suspension and to fully digest the second liquid phase. This manner of crystallization generated highly agglomerated particles.

An unseeded isothermal crystallization in which no liquid-liquid phase separation occurred was performed to allow for comparison. 19 ml of pembrolizumab drug substance solution with a concentration of about 165 mg/ml was charged to a reactor equipped with an overhead stirrer. A solution was prepared in an appropriate separate container by dissolving 3100 mg of DSS, 1500 mg of PEG3350, 460 mg of caffeine in 48 ml water and 6.6 ml of HEPES buffer (pH 7.3) and was charged to reactor. The mixture was aged under agitation over no less than 48 h after the point nucleation at 5° C. to produce a crystalline suspension. This manner of crystallization afforded non-agglomerated, platelet-like particles.

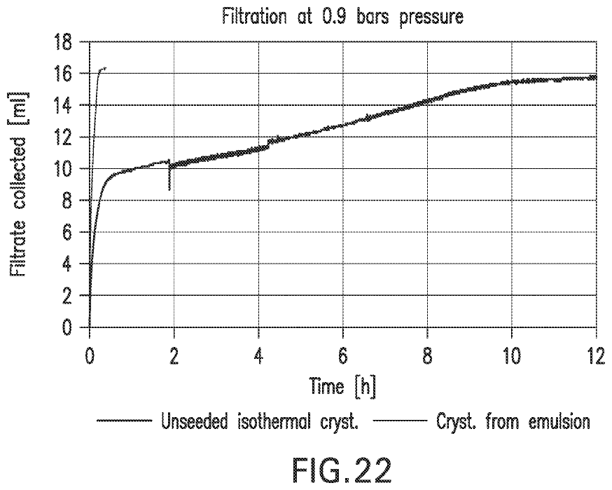

Approximately 20 ml of crystalline suspension were filtered on a pressure filter with an inner diameter of 35 mm with a mixed cellulose ester filter with a pore size 0.45 microns 0.9 bars. FIG. 22 shows the collected filtrate over time.

As during the filtration of the highly agglomerated particles more filtrate was collected within a shorter period, it can be concluded that highly agglomerated particles crystallized from an emulsion filtered faster and deliquored more efficiently than non-agglomerated ones crystallized from a monophasic solution.

### Example 17

### Impact of Varying Ultrafiltration Product (UFP) Concentration on Pembrolizumab Crystallization Purity and Yield

Approximately 60-71 mLs of ultrafiltration product (UFP) at a concentration of 176-282 g/L in approximately 100 mM L-histidine, 10 mM L-methionine pH 5.5 was placed in an EasyMax reactor with agitation at 25° C. The following components were added to the UFP to start the crystallization: caffeine at final concentration of ~1.7 g/L, PEG3350 at final concentration of ~4.0% (w/v), arginine at a final concentration of ~9.6 g/L, and sodium chloride at a final concentration of ~0.07 g/L. Crystallization time was at least 12 hours at 25° C.

After crystallization was finished, a 1 mL aliquot of crystalline slurry was taken to measure higher order aggregation by analytical ultra performance size exclusion chromatography (UP-SEC). A portion of the 1 mL crystalline slurry was diluted using 100 mM Histidine 10 mM Methionine pH 5.5 for sample preparation for UP-SEC assay. To analyze for pembrolizumab concentration in the supernatant, a 1 mL aliquot of the crystalline slurry was taken and centrifuged at least ~12,000 rpm for 30 minutes. The clear supernatant was at the top of the tube with crystalline pembrolizumab packed at the bottom of the centrifuge tube. An aliquot of clear supernatant was taken and placed in a vial and analyzed by analytical Protein A chromatography. Crystallization yield is calculated as the (1-(pembrolizumab amount in the supernatant/pembrolizumab amount in the UFP))×100.

The impact of pembrolizumab concentration on the crystallization yield and UP-SEC purity was examined using a range of 176-282 g/L of pembrolizumab in UFP. Results indicate the UP-SEC purity and yield does not significantly change up to 282 g/L pembrolizumab UFP concentration.

TABLE 15
UPSEC Purity and Crystallization Yield for
UF Feed and UFP at Different Concentrations

| Sample | UPSEC Purity (A %) | Crystallization Yield (%) |
|---|---|---|
| UF feed | 99.6 | — |
| UFP at 176 g/L | 99.6 | 98.2 |
| UFP at 209 g/L | 99.5 | 98.3 |
| UFP at 282 g/L | 99.6 | 99.0 |

### Example 18

### Impact of Amino Acids on Pembrolizumab Ultrafiltration Product (UFP) Concentration and Crystallization

Approximately 300 mL of ultrafiltration product (UFP) at a concentration of 70 g/L in 10 mM Histidine, 10 mM Methionine pH 5.5 was loaded into the tangential flow filtration (TFF) system. The TFF system was setup using an Ultracel 30 kDa membrane D Screen, with a TMP setpoint of 15 psi, and a feed flow rate from 7 to 28 L/min*m2. UFP for each run was then concentrated to 100 g/L before the material was diafiltered into the corresponding buffer for each run, up to 7 Diavolumes. After diafiltration step, as each run was being further concentrated, the TFF system's throttling valve was allowed to automatically adjust as the system's pressure rose until the throttling valve was fully open. After the throttling valve was fully open the feed flow rate was adjusted manually keeping the system feed pressure below 30 psi by reducing the flow, this was done until the pump reached its lowest capacity 5-10 mL/min. The final UFP was then removed from the system through the retentate line into a bottle. Throughout each experiment, samples were taken throughout the final concentration step to collect viscosity data at various concentrations.

The first condition using 100 mM L-histidine 10 mM L-methionine was run on a single 88 cm² membrane, after the completion of the diafiltration and concentration the final UFP was removed from the system, there was then a second membrane added for a total of 176 cm² and further concentration was conducted. For the second condition, 150 mM Arginine, was run on 176 cm² of membrane and then the final UFP removed, then following a system flush and cleaning cycle the UFP was returned to the system for further concentration. For the final condition 150 mM Lysine it was run once on 176 cm² membrane area.

The crystallization of each UFP stream was set-up as described in the following table and run at 25° C.

TABLE 16
Cystallization experiments for each UFP stream in experiments 1-6

| | UFP | | | | |
|---|---|---|---|---|---|
| | Concen- | Exper- | | | |
| UF Diafiltration | tration | iment | PEG3350 | Caffeine | UFP |
| Buffer | (g/L) | Number | (w/v %) | (g/L) | pH |
| 10 mM L-histidine | 213 | 1 | 0 | 1.7 | 5.4 |
| 100 mM L-histidine | 268 | 2 | 0 | 1.7 | 5.6 |
| | | 3 | 4.5 | 1.7 | 5.7 |
| 150 mM arginine | 225 | 4 | 4.5 | 1.7 | 5.9-6.3 |
| | | 5 | 4.5 | 3.4 | 5.9 |
| 150 mM lysine | 219 | 6 | 4.5 | 1.7 | 5.5 |

*Histidine samples contain 10 mM L-methionine except for UF feed.

After crystallization was finished, a 1 mL aliquot of crystalline slurry was taken to measure higher order aggregation by analytical ultra performance size exclusion chromatography (UP-SEC). A portion of the 1 mL crystalline slurry was diluted using the corresponding diafiltration buffer for sample preparation for UP-SEC assay. To analyze for pembrolizumab concentration in the supernatant, a 1 mL aliquot of the crystalline slurry was taken and centrifuged at least ~12,000 rpm for 30 minutes. The clear supernatant was at the top of the tube with crystalline pembrolizumab packed at the bottom of the centrifuge tube. An aliquot of clear supernatant was taken and placed in a vial and analyzed by analytical Protein A chromatography. Crystallization yield is calculated as the (1-(pembrolizumab amount in the supernatant/pembrolizumab amount in the UFP))×100.

L-histidine at 100 mM achieved the highest pembrolizumab UFP concentration when compared to 150 mM lysine and 150 mM arginine at pH 5.4 in the diafiltration buffer. The UPSEC purity remained consistent after UF step and UF yield was >80% across all experiments. Increasing the L-histidine concentration from 10 to 100 mM in the diafiltration buffer increased the UFP concentration from 213 g/L to 268 g/L.

TABLE 17
Impact of amino acids on UFP concentration,
UPSEC purity and UFP yeild

| | UFP Concentration | UPSEC Purity | UFP Yield |
|---|---|---|---|
| Sample | (g/L) | (A %) | (%) |
| 10 mM L-histidine | 213 | — | — |
| 100 mM L-histidine | 268 | 98.5 | 82 |
| 150 mM arginine | 225 | 99.0 | 95 |
| 150 mM lysine | 219 | 99.0 | 94 |

*Histidine samples contain 10 mM L-methionine except for UF feed. UP-SEC purity of UF feed was 98.9 A %.

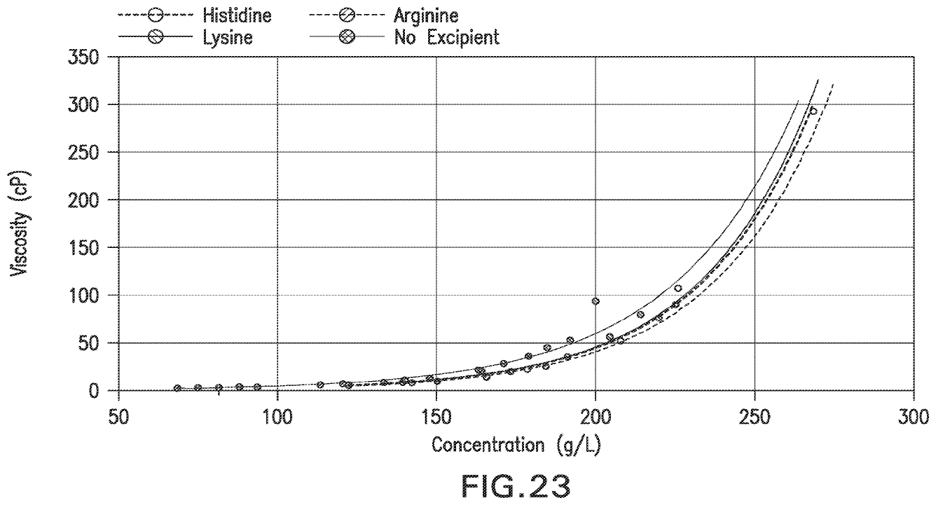

As shown in FIG. 23, UFP viscosity for 150 mM arginine experiment was slightly lower than 150 mM lysine and 100 mM L-histidine experiments. However, viscosity did not impact the final concentration as the 100 mM L-histidine reached highest concentration at 268 g/L.

PEG3350 is needed to decrease the pembrolizumab concentration in the supernatant and maintain >90% yield. As the histidine concentration increased in the UFP, the pembrolizumab solubility increased supernatant from 25 g/L to 120 g/L without any PEG3350 addition. After adding 4 (w/v) % PEG3350 to the crystallization, pembrolizumab solubility in the crystallization supernatant decreased from 120 g/L to 0.8 g/L in the 100 mM L-histidine experiment.

TABLE 18
Impact of amino acids on crystallization concentration, yield, pH and UP-SEC purity

| | | | | | | UP- |
|---|---|---|---|---|---|---|
| | | Crystallization | Supernatant | | | SEC |
| | Experiment | Concentration | Concentration | Crystallization | Crystallization | Purity |
| Sample | Number | (g/L) | (g/L) | Yield (%) | pH | (A %) |
| 10 mM | 1 | 198 | 25 | 87 | 6.3 | 99.0 |
| L- | | | | | | |
| histidine | | | | | | |
| 100 mM | 2 | 240 | 120 | 50 | — | — |
| L- | 3 | 225 | 0.03 | 100 | 5.5 | 98.5 |
| histidine | | | | | | |
| 150 mM | 4 | 190 | 0.37 | 100 | 6.0-6.5 | 99.0 |
| arginine | 5 | 174 | 0.4 | 100 | — | — |
| 150 mM | 6 | 185 | 0.6 | 100 | 5.5 | 99.1 |
| lysine | | | | | | |

* Histidine samples contain 10 mM L-methionine.

### Example 19

### Minipig Pharmacokinetic Profile Comparison of Liquid and Crystalline Suspension Formulations of Pembrolizumab

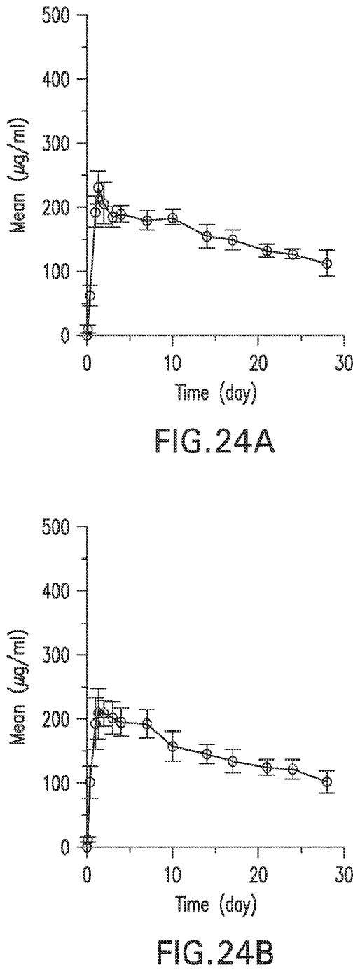

A minipig pharmacokinetic (PK) study was designed to compare the relative PK profiles and bioavailability of liquid (165 mg/mL pembrolizumab, 70 mg/mL sucrose, 0.2 mg/mL Polysorbate 80, 10 mM L-Methionine, 10 mM Histidine pH 5.5) and crystalline suspension (165 mg/mL pembrolizumab, 50 mM L-Arginine HCl, 50 mM sodium chloride, 70 mg/mL sucrose, 0.2 mg/mL Polysorbate 80, 10 mM L-Methionine, 10 mg/mL PEG 3350, 1.4 mg/mL caffeine, 7.7 mM Histidine, 20 mM HEPES, pH 6.0) formulations of pembrolizumab after subcutaneous injection. For both formulations the minipigs were injected with 1.0 mL of sample was injected per minipig (n=5 minipigs) in the inner thigh region. Analysis of the PK dosing groups indicated relative bioavailability of liquid is similar to anti-PD-1 crystalline suspension (FIGS. 24A and 24B, Table 19). Based on the concentration-time profiles, no indication of fast irregular clearance was detected in these two dosing groups.

TABLE 19
Pharmacokinetics parameters of pembrolizumab (human anti-
PD-1) administrated subcutaneously in female minipigs using
crystalline formulation and liquid formulation.

| | Crystalline | Liquid | Liquid |
|---|---|---|---|
| | Formulation | Formulation | Formulation |
| Formulation | (SC) | (SC) | (IV) |
| Cₘₐₓ/Dose (μg/mL) | 219.560 | 231.200 | 405.280 |
| Tₘₐₓ (day) | 1.33 | 1.33 | 0.067 |
| AUC_{inf} (h*μg/mL) | 8345.996 | 9951.332 | 8639.890 |
| % Bioavailability | 97 | 115 | 100 |

AUC: area under the curve

1. A method for producing a crystalline suspension of an anti-programmed death 1 (anti-PD-1) monoclonal antibody (mAb) comprising:
(a) mixing an aqueous crystallization solution comprising:
i. an aqueous buffered solution comprising about 150 mg/ml to about 300 mg/ml of the mAb, wherein the anti-PD-1 mAb is pembrolizumab or a pembrolizumab variant, and
ii. an additive selected from the group consisting of caffeine, theophylline, 2′ deoxyguanosine-5′-monophosphate, a bioactive gibberellin, and a pharmaceutically acceptable salt of said bioactive gibberellin,
to form a crystallization solution, wherein the crystallization solution has a pH of about 5.0 to about 8.0;
(b) incubating the crystallization solution for a period of time sufficient to form the crystalline suspension.

2. (canceled)

3. The method of claim 1, wherein the aqueous buffered solution further comprises a buffer selected from histidine, lysine, arginine, or HEPES.

4. The method of claim 1, wherein the aqueous buffered solution further comprises histidine buffer at a pH of about 5.0 to about 7.0.

5. The method of claim 1, wherein the aqueous buffered solution comprises 10-100 mM of a buffer selected from histidine, lysine, arginine, or HEPES.

6. The method of claim 1, wherein the additive is caffeine.

7. The method of claim 6, wherein the additive is about 0.10% to about 0.30% w/v caffeine.

8. The method of claim 1, wherein the additive is about 0.25% to about 0.30% w/v theophylline.

9. The method of claim 1, wherein the additive is caffeine, and the crystallization solution further comprises about 1% to about 10% w/v dextran sodium sulfate (DSS).

10. (canceled)

11. The method of claim 1, wherein the method further comprises mixing PEG with the aqueous buffered solution and the additive to form the crystallization solution.

12-14. (canceled)

15. The method of claim 11, wherein the PEG is present in the crystallization solution in an amount of about 0% to about 30% w/v.

16-17. (canceled)

18. The method of claim 11, wherein the concentration of PEG is 10 mg/ml.

19. The method of claim 11, wherein
the pH of the crystallization solution and the amount of PEG present in the crystallization solution is selected from the group consisting of:
a) pH of the crystallization solution is less than 6.0 and the amount of PEG is 2-6% w/v,
b) pH of the crystallization solution is about 6.0 and the amount of PEG is 0-4% w/v,
c) pH of the crystallization solution is about 6.2 and the amount of PEG is 0-4% w/v,
d) pH of the crystallization solution is about 6.4 and the amount of PEG is 0-4% w/v, and
e) pH of the crystallization solution is from about 6.8 to about 8.0 and the amount of PEG is 0-2% w/v.

20. (canceled)

21. The method of claim 11, wherein the molecular weight of the PEG is from about 2,500 to about 20,000.

22. The method of claim 1, wherein the crystallization solution is incubated at an incubation temperature of from about 2° C. to about 40° C.

23. (canceled)

24. The method of claim 1, wherein the crystallization solution is heated to about 50° C., then post-crystallization cooled to a temperature of about 37° C. or lower.

25-27. (canceled)

28. The method of claim 1, wherein the crystallization solution is incubated for about 15 minutes or more.

29-30. (canceled)

31. The method of claim 1, wherein the concentration of the anti-PD-1 mAb in the crystallization solution is from about 170 mg/mL to about 300 mg/mL.

32-35. (canceled)

36. The method of claim 1, wherein the concentration of the anti-PD-1 mAb in the crystallization solution is about 165 mg/mL.

37. (canceled)

38. The method of claim 1, wherein the crystallization solution further comprises from about 5 mM to about 100 mM histidine, arginine, lysine, or HEPES buffer.

39-41. (canceled)

42. The method of claim 1, wherein the anti-PD-1 mAb is pembrolizumab.

43-60. (canceled)

61. A pembrolizumab crystal produced by claim 1, wherein at least one distinct phase of the crystalline solution is identified by one of the following:
(i) 181.90, 181.40, 180.36, 179,69, 137.00, 135.17, 109.28, 108.12, 66.88 and 65.35, 40.69, 27.66, 27.24, 20.75 ppm, as identified by solid-state NMR,
(ii) 0.55, 0.71, 0.90, 1.10, 1.16, 1.20, 1.41, 1.51, 1.55, 1.58, 1.69, 1.76, 1.79, 1.86, 1.93, 2.00, 2.08, 2.17, 2.20, 2.27, 2.34, 2.40, 2.42, 2.52, 2.63, 2.70, 2.72, 2.74, 2.78, 2.81, 2.86, 2.91, 2.95 nm⁻¹, as identified by synchrotron SAXS,
(iii) 0.55, 0.90, 1.10, 1.41, 1.52, 1.69, 1.84, 1.93, 2.28 nm⁻¹ as identified by laboratory SAXS,
(iv) 182.16, 181.54, 180.59, 179,99, 137.16, 135.43, 109.36, 108.23, 66.97 and 65.57, 40.80, 27.50, 27.01, 20.85 ppm, as identified by solid-state NMR,
(v) 0.36, 0.60, 0.71, 0.93, 1.20, 1.42, 1.45, 1.50, 1.62, 1.68, 1.79, 1.82, 1.88, 1.99, 2.02, 2.06, 2.18, 2.19, 2.30, 2.44, 2.50, 2.58, 2.72, 2.79, 2.86, 2.88, 2.92, 2.98, 3.03, 3.09 nm⁻¹, as identified by synchrotron SAXS, or
(vi) 0.60, 0.93, 1.20, 1.44, 1.81, 2.02, 2.20 nm⁻¹, as identified by laboratory SAXS.

62-63. (canceled)
